SEQUENCE LISTING (75 sequences)

SEQ ID NO: 1 - DNA, 443 bp, Homo sapiens
  misc_feature (1)..(443): n=a, t, g, or c
cgcaccggnnnnccggttgacccacgcgtccgcgctccctctctctgccaccgctcaaagcctgggataacgcttttgaaagcgaatcaggaaattacttaggaatcggaagccccaaagaattatgaataatcctcgctgccaaagggaaggggattttgagcaaaagctccacatctgcgcacactagagttcaaagactccagctgttggaaggtcttgtgagcaatgtttgagaggtaagactggaccgctaggtcttgccggtgagaaaggggaccaaggaaagactgggaagaaaggacccatatgaccatagggagagaaaggagaagtaggtccaattggtcctcctggacccaagggagacagaggagaacaaggggaccccgggctgcctggggttttgccgatgtggaagcatcctggctcaaatccggctc

SEQ ID NO: 2 - DNA, 1397 bp, Homo sapiens
ctcagagcggtggaattcgaacgcttttgaaagcgaatcaggaaattacttatcaatctgaagccccatataattatgaataatcctcgctgccaaagggaaggggattttgagcaaaagctccacatctgcgcacactagagttcaaagactccagctgttggaaggtcttgtgagcaagagccaaagatgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtgatcaggaccaagatccctgtggtaaacactctgattgaatctggggttccagaaggtggaacaagcaggaatgggatccaaagagactcccactcagattctaaagcatttaaagacaattctagcagaatttatcaaaacaagatgaaacacagaaaagttgaaaccacaacaaaatgaattctattaaagaatagccccagatataaattctcttgaaagcaatgttcataaatatttaagcaaattaaagacaatgttaacaaattttctattaaatgccctgagtgataaaaccagttggcaataatattgccttattaaatcttcaaaaaataa

SEQ ID NO: 3 - DNA, 1297 bp, Homo sapiens
  CDS (80)..(949)
cgcccggaattcccgggtcgaggcacgcgttcgatttagaatcctgcagcaccccaccatctaagagcaagagccaaagatgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtgatcaggaccaagatccctgtggtaaacactctgattgaatctggggttccagaaggtggaacaagcaggaatgggatccaaagagactcccactcagattctaaagcatttaaagacaattctagcagaatttatcaaaacaagatgaaacacagaaaagttgaaaccacaacaaaatgaattctattaaagaatagccccagatataaattctcttgaaagcaatgttcataaatatttaagcaaattaaagacaatgttaacaaattttctattaaatgccctgagtgataaaaccagttggcaataatattgccttattaaatcttcaaaaaataaaaaaaaaaaa

SEQ ID NO: 4 - protein, 289 aa, Homo sapiens
MFVLLYVTSFAICASGQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 5 - DNA, 870 bp, Homo sapiens
atgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 6 - protein, 16 aa, Homo sapiens
MFVLLYVTSFAICASG

SEQ ID NO: 7 - DNA, 819 bp, Homo sapiens
ccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 8 - protein, 273 aa, Homo sapiens
QPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 9 - DNA, 963 bp, Homo sapiens
atgtttgtcttgctgtatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctccaggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgagtctagagggcccgcggttcgaaggtaagcctatccctaaccctctcctcggtctcgattctacgcgtaccggtcatcatcaccatcaccattga

SEQ ID NO: 10 - protein, 320 aa, Homo sapiens
MFVLLYVTSFAICASGQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDELSLEGPRFEGKPIPNPLLGLDSTRTGHHHHHH

SEQ ID NO: 11 - DNA, 20 bp, Artificial Sequence
  PCR primer sequence
agggaactgcaggtttgaga

SEQ ID NO: 12 - DNA, 20 bp, Artificial Sequence
  PCR primer sequence
tcctctgtctccctttggtc

SEQ ID NO: 13 - DNA, 20 bp, Artificial Sequence
  PCR primer sequence
tgtccagacaagagccaaag

SEQ ID NO: 14 - DNA, 19 bp, Artificial Sequence
  PCR primer sequence
tgggtagctggttgtgatg

SEQ ID NO: 15 - protein, 289 aa, Homo sapiens; identical to SEQ ID NO: 4

SEQ ID NO: 16 - DNA, 1290 bp, Mus musculus
aggaaagatcctgacttttgtacactgggaatcctgcagcaacctaccctcccagaacacgagcccaagatgattgtcctgctctacgtgacgagtcttgccatctgtgcaagtggacaacctcgggccaatcaggctaagggagagagctactctccaaggtacatctgcagcatccctggattacctgggcccccaggtcctcctggagcaaatggctcccctgggccccatggtcgcattggccttcctggaagggatggtagagatggcagaaaaggagagaagggggaaaagggcactgcaggtctaaaaggtaaaactggacccctgggccttgctggtgagaaaggagaccaaggagaaactgggaagaaaggacccataggaccagagggtgagaaaggagaagtcggtccagctgggcctcctgggccaaagggagacagaggagatcaaggggacccagggctgcctggagtgtgcaggtgtggaagcattgtgctcaaatctgccttttcagttggcatcacaaccagctacccagaagaaagactacccatcatatttaacaaagtcctcttcaatgagggggagcattacaaccctgcaacggggaagttcatttgcgctttcccagggatctattacttttcttatgacatcacgttggccaataagcacctagcaatcgggctggtgcacaatgggcagtaccggataaggacctttgatgccaacacagggaaccatgatgtggcgtcggggtccacagtcatctacctgcagccagaagatgaggtctggctggagatcttcttcaatgaccagaacggcctcttctcagatccaggctgggcagacagcttgttctctgggtttctcctctatgtcgatacagattacctggattctatatcagaggatgatgagctgtgatccagaccactacaggcctgaatgttgcaaacatgagtaccacagtggctgacactctaatctggagtgctggaaggtggagcaagtgatacggggattcagaaaacgttttttacagacgactcaggctgagttatcaaaataagacaaaccaccaactagctgaaatcacaacaaaacgaatggcatacaataacctcagacatggaccccctaaagtaatgatcctaaatattgaagcaaattaaagcaaatgatgttaacaaatttgaatgcccttggcaatacaaccagctggaaatgacactgcctcattaaatattcataaaaccccaaaaaaaaaaaaaaaaa

SEQ ID NO: 17 - protein, 289 aa, Mus musculus
MIVLLYVTSLAICASGQPRANQAKGESYSPRYICSIPGLPGPPGPPGANGSPGPHGRIGLPGRDGRDGRKGEKGEKGTAGLKGKTGPLGLAGEKGDQGETGKKGPIGPEGEKGEVGPAGPPGPKGDRGDQGDPGLPGVCRCGSIVLKSAFSVGITTSYPEERLPIIFNKVLFNEGEHYNPATGKFICAFPGIYYFSYDITLANKHLAIGLVHNGQYRIRTFDANTGNHDVASGSTVIYLQPEDEVWLEIFFNDQNGLFSDPGWADSLFSGFLLYVDTDYLDSISEDDEL

SEQ ID NO: 18 - DNA, 2186 bp, Mus musculus
aggaaagatcctgacttttgtacactgggaatcctgcagcaacctaccctcccagaacacgagcccaagatgattgtcctgctctacgtgacgagtcttgccatctgtgcaagtggacaacctcgggccaatcaggctaagggagagagctactctccaaggtacatctgcagcatccctggattacctgggcccccaggtcctcctggagcaaatggctcccctgggccccatggtcgcattggccttcctggaagggatggtagagatggcagaaaaggagagaagggggaaaagggcactgcaggtctaaaaggtaaaactggacccctgggccttgctggtgagaaaggagaccaaggagaaactgggaagaaaggacccataggaccagagggtgagaaaggagaagtcggtccagctgggcctcctgggccaaagggagacagaggagatcaaggggacccagggctgcctggagtgtgcaggtgtggaagcattgtgctcaaatctgccttttcagttggcatcacaaccagctacccagaagaaagactacccatcatatttaacaaagtcctcttcaatgagggggagcattacaaccctgcaacggggaagttcatttgcgctttcccagggatctattacttttcttatgacatcacgttggccaataagcacctagcaatcgggctggtgcacaatgggcagtaccggataaggacctttgatgccaacacagggaaccatgatgtggcgtcggggtccacagtcatctacctgcagccagaagatgaggtctggctggagatcttcttcaatgaccagaacggcctcttctcagatccaggctgggcagacagcttgttctctgggtttctcctctatgtcgatacagattacctggattctatatcagaggatgatgagctgtgatccagaccactacaggcctgaccacgcgtccgcccacgcgtccgcccacgcgtccgcggagcaaagacgtcggatggccctggacgtggctcggggcacagaagcaagaagacttgatgaagcctttcttcccaacccattcccagaaagaacaacttagatgacaatttttaaaaaggtgacaaccatgatctcctgggtactcttggcctgtgcccttccgtgtgctgctgacccaatgcttggtgcctttgctcgcagggacttccagaaggggggtcctcaactagtttgcagcctgcctggtccccaaggcccacctggccctccaggagcaccaggatcctcaggagtggtgggaagaatgggtttccctgggaaagacggccaagatggccaggacggagaccggggggacagtggagaagaaggtccacctggcaggacaggcaaccgtggaaaacaaggaccaaagggcaaagctggggccattggcagagctggccctcgaggacccaagggggtcagtggtacccccgggaagcatggcacaccgggcaagaagggacctaagggcaaaaaaggggagcctggcctcccaggcccctgcagctgcggcagtagccgagccaagtcggccttttccgtggcggtaaccaagagctacccacgcgagcgactgcctatcaagtttgacaagattctgatgaacgagggtggccactacaacgcgtccagtggtaagttcgtctgcagcgtgccggggatctattactttacctatgacattacgctggccaacaaacacctggccatcggcctggtgcacaatggtcaataccgcattcggacttttgatgccaacacgggcaaccacgacgtggcctcgggctccaccatcctagctctcaaggagggtgatgaagtctggctgcagatcttctactcagagcagaatggcctcttctacgacccttactggaccgacagcctgttcaccggcttcctcatctacgctgaccaaggagaccccaacgaggtatagacaagccggggttgagccttgaggtagggactaagagtctgcgtgggtgcctggaggaagattccctcgactggggctgtggactgacaatcttgggagcttttattcccaggcaggcctcctctattgctgctttaaaaaaaatcattaaatccaagctattgattcatctatttctctgtccatcaaaaaaaaaaaaaaa

SEQ ID NO: 19 - protein, 285 aa, Mus musculus
MISWVLLACALPCAADPMLGAFARRDFQKGGPQLVCSLPGPQGPPGPPGAPGSSGVVGRMGFPGKDGQDGQDGDRGDSGEEGPPGRTGNRGKQGPKGKAGAIGRAGPRGPKGVSGTPGKHGTPGKKGPKGKKGEPGLPGPCSCGSSRAKSAFSVAVTKSYPRERLPIKFDKILMNEGGHYNASSGKFVCSVPGIYYFTYDITLANKHLAIGLVHNGQYRIRTFDANTGNHDVASGSTILALKEGDEVWLQIFYSEQNGLFYDPYWTDSLFTGFLIYADQGDPNEV

SEQ ID NO: 20 - DNA, 1056 bp, Rattus norvegicus
atggcttcagctcacgatgtggaatactcagctaccatcacctcatccccaccacaactctcactcactcgtgtcagcttacagtcagctcagaagcaagtttttcaccgagctctcaggaaagatcctgacttttgtacactgggaatcctgcagcaacctaccctcccagagcacgagcccaagatgattgtcctgctctacgtgacaagtcttgccatctgtgcaagtggacaacctcggggcaatcaggctaagggggagagctactctccaaggtacatctgcagcatccctggcttacctgggcccccaggtcctcctggagcaaatggctcccctggaccccatggtcgcattggccttcctggaagggatggtagagatggcagaaaaggagagaagggggaaaagggcactgcaggtctaaaaggtaagactggacccctgggccttgctggtgagaaaggagaccaaggagagactgggaagaaaggacccataggaccagagggtgagaaaggagaagttggtccagctgggcctcctgggccaaagggagaccgaggagatcaaggggacccaggactgcctggagtgtgcagatgtggaagcatcgtgctcaaatctgccttttcagttggcatcacaaccagctacccagaagaaagactacccatcatatttaacaaagtcctcttcaatgagggggagcactacaaccctgcaacagggaagttcatttgtgctttcccagggatctattatttttcttatgatatcacgttggccaataagcacctagccatcgggctggtgcacaatgggcagtaccggataaggacctttgatgccaacacagggaaccacgatgtggcttcagggtccacagtcatctacctgcagccagaagatgaggtctggctggagattttcttcaatgaccagaatggcctcttctcagatccaggctgggcagacagcttgttctccgggtttctcctctatgttgatacagattacctggattctatatcagaagacgatgagttgtga

SEQ ID NO: 21 - protein, 289 aa, Rattus norvegicus
MIVLLYVTSLAICASGQPRGNQAKGESYSPRYICSIPGLPGPPGPPGANGSPGPHGRIGLPGRDGRDGRKGEKGEKGTAGLKGKTGPLGLAGEKGDQGETGKKGPIGPEGEKGEVGPAGPPGPKGDRGDQGDPGLPGVCRCGSIVLKSAFSVGITTSYPEERLPIIFNKVLFNEGEHYNPATGKFICAFPGIYYFSYDITLANKHLAIGLVHNGQYRIRTFDANTGNHDVASGSTVIYLQPEDEVWLEIFFNDQNGLFSDPGWADSLFSGFLLYVDTDYLDSISEDDEL

SEQ ID NO: 22 - DNA, 1173 bp, Rattus norvegicus
  misc_feature (1)..(1173): n = g, a, t, or c
caagctgctgcaagcaggactgagcactaccacagcagcaacctcggatggccctggacgtggcacgcgcggggcacagaggcaagaagacttgatgaagcctctcttcccaacccatatccagaaagaacgatttagatgacagtttttagaaaggtgaccaccatgatctcctggatgctcttggcctgtgcccttccgtgtgctgctgacccaatgcttggtgcctttgctcgcagggacttccagaagggtggtcctcaactggtgtgcagtctgcctggtccccaaggcccacctggccctccaggagcaccaggatcctcaggaatggtgggaagaatgggttttcctggtaaggatggccaagacggccaggacggagaccgaggggacagtggagaagaaggtccacctggcaggacaggcaaccgaggaaaacaaggaccaaagggcaaagctggggccattgggagagcgggtcctcgaggacccaagggggtcagtggtacccccgggaaacatggtataccgggcaagaagggacctaagggcaagaaaggggaacctgggctcccaggcccctgtagctgcggcagtagccgagccaagtcggccttttcggtggcggtaaccaagagttacccacgtgagcgactgcccatcaagtttgacaagattctgatgaatgagggaggccactacaatgcatccagtggcaagttcgtctgcagcgtgccagggatctattactttacctatgacattacgctggccaacaaacacctggccatcggcctagtgcacaatggccagtaccgcattcggacttttgacgccaacaccggcaaccacgacgtggcctcgggctccaccatcctagctctcaaggagggtgatgaagtctggttacagattttctactcggagcagaatggactcttctacgacccttattggaccgacagcctgttcaccggcttcctnatctacgctgatcaaggagaccccaatgaggtatagacaagctggggttgagcgtccaggcagggactaagattccgcaagggtgctgatagaagaggatctctgaactgaggctggggactggcagtcttgggagcttttattcccaggcaagcctcctctgttgctgctttaaaaaaaaaaaaa

SEQ ID NO: 23 - protein, 285 aa, Rattus norvegicus
  MISC_FEATURE (1)..(285): X = any amino acid
MISWMLLACALPCAADPMLGAFARRDFQKGGPQLVCSLPGPQGPPGPPGAPGSSGMVGRMGFPGKDGQDGQDGDRGDSGEEGPPGRTGNRGKQGPKGKAGAIGRAGPRGPKGVSGTPGKHGIPGKKGPKGKKGEPGLPGPCSCGSSRAKSAFSVAVTKSYPRERLPIKFDKILMNEGGHYNASSGKFVCSVPGIYYFTYDITLANKHLAIGLVHNGQYRIRTFDANTGNHDVASGSTILALKEGDEVWLQIFYSEQNGLFYDPYWTDSLFTGFXIYADQGDPNEV

SEQ ID NO: 24 - DNA, 1347 bp, Bos taurus
  misc_feature (1)..(1347): n =g , a, t, or c
cggctcgaggcggaccagaactttatttttcatcaagctattttttaagttgttttgaagccgcttcaaataataaacacatatttctgctggaaacattgtttaatggtttctgaattcatgggacaagagaagcaagaaagcctcatgatttggtggaaggtttgatatcagcaatgcctagaaaagagccaaagatgtttgtcttgctctatgttacaagttttgccatttgcgccagtggacaaccgcggagtaatcagttcaaaggagagagctactccccaaggtatatctgtagcattcctggcctgcctgggcctccaggcccccctggagcgaatggttcccccgggccccatggtcgcatcggccttccaggacgagatggtagagacggcaggaaaggagagaaaggtgaaaagggggccgcaggtttgagaggtaaggctggacccctgggccttgctggagagaaaggggaccaaggagagactgggaagaagggacccatgggacctgagggagagaaaggagaagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgccaacacaggaaaccatgacgtggcttcagggtccacagtcatctatctgcagccagaagatgaagtctggctggagatattcttcactgaccagaatgggctcttctcagacccaggttgggctgacagcttgttctccggatttctcctgtatgttgacacagattatctagactccatatcagaagacgatgagctctgatgaggactgctggcttgaatgtcccaagatccttatggcagacactttgatttaatctggggccctggaagttggaaggagcggaaatgagatccaaaaatactcccactcagattccagagcatttacagacagttctagcagaatccgttaaaacaagttgaaccaccaaaccattgaaaccacactaaaatgaattatataaaacaaaaatccagatatgttctctcaaaagtgatgttcagaaatatttaaacaaattaaagacaatgtttcaaaa

SEQ ID NO: 25 - protein, 296 aa, Bos taurus
  MISC_FEATURE (1)..(296): X = any amino acid
MPRKEPKMFVLLYVTSFAICASGQPRSNQFKGESYSPRYICSIPGLPGPPGPPGANGSPGPHGRIGLPGRDGRDGRKGEKGEKGAAGLRGKAGPLGLAGEKGDQGETGKKGPMGPEGEKGEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXANTGNHDVASGSTVIYLQPEDEVWLEIFFTDQNGLFSDPGWADSLFSGFLLYVDTDYLDSISEDDEL

SEQ ID NO: 26 - DNA, 1039 bp, Bos taurus
  misc_feature (1)..(1039): n = g, a, t, c
ccacgcgtccgcagacgcccctggacgtggccggcggcggtgagggtggccaccatgatcccctgggtgctcctggcctgtgcccttccttgtgcggccgacccactgctggctgcctttgcccgtagagacttccagaagggctcccctcaactcatctgcagcatgcctggcccccagggtccacccggccccccaggagccccagggccctcaggaatggtgggaagaatgggctttccaggcagagatggccaggatggccaggacggggaccgaggggagaccggagaggaaggttcacctggccggacaggtaaccgggggaagccaggcccgaagggcaaagctggggccattgggcgggccggcccacgtggccccaaaggtgtcagcggtgccccgggcaagcacggcacacctggcaagaaggggcccaagggcaagaagggggagccaggcctcccggggccctgcagctgcggnagcagccacgccaagtcggccttntncgtggcagtgaccaaaaactatnctcgggagcggttgccnattaaattnnnnnnnnnnnnnnnnnnnnnnnnnnnnnnnnnnnnnnnnnnnnnnnnnnnnnnnnnnnnnnnngtgccgggncattactacttcacntacgacatcacgctggccaacaagcacctggccatcggcctggtgcacaatggccagtatngcatccggaccttcgacgccaacacgggcaaccacgacgtggcctcgggctccaccatcctggcgctcaggcagggggacgaggtctggctgcagatcttctactctgagcagaatgggctcttttacgacccatactggactgacagcctcttcacgggcttcctcatctatgccgaccaggacaaccccagtgaggtgtagacgggacagttctgggtctcccagctgctcctggacttgggcttccagcacaagacccctaaactgttgggagctggaagtcaggagcatctaacctcaggccaacctcctccacccgtttttcgtc

SEQ ID NO: 27 - protein, 285 aa, Bos taurus
  MISC_FEATURE (1)..(285): X = any amino acid
MIPWVLLACALPCAADPLLAAFARRDFQKGSPQLICSMPGPQGPPGPPGAPGPSGMVGRMGFPGRDGQDGQDGDRGETGEEGSPGRTGNRGKPGPKGKAGAIGRAGPRGPKGVSGAPGKHGTPGKKGPKGKKGEPGLPGPCSCXSSHAKSAXXVAVTKNYXRERLXIKXXXXXXXXXXXXXXXXXXXXXXVPXHYYFXYDITLANKHLAIGLVHNGQYXIRTFDANTGNHDVASGSTILALRQGDEVWLQIFYSEQNGLFYDPYWTDSLFTGFLIYADQDNPSEV

SEQ ID NO: 28 - DNA, 1463 bp, Sus scrofa
  misc_feature (1)..(1463): n = g, a, t, or c
tgcaaaacttcggacgacagcatgagctccaagcttcaaaaggattctcacgcgcggaggagcagtttatttttcatcaagttatcttccgtgtctttttgaagcagcttcgaagcataaacacctatttctgctttaaacttttttaatggtttctaaattcatgggacaaccaaagccagaaagcctcacgctttggaggagcatttgatgtcagcaatgcctagaaaagagccaaagatgtttgtcttgctctatgttacaagttttgccatttgtgcaagtggacaacctcggggtaatcagttcaaaggcgatacctactccccgagatatatctgtagcattcctggcttacctggacctccaggcccccctggagcaaatggttcccctgggccccatggtcggatcggccttccgggacgagatggtagagacggcaggaaaggagaaaaaggtgaaaagggggctgcaggtttgagaggtaagactggaccactgggccttgcnnnnnnnnnnnnnnnnnnnnnnnnnnnnnnnnnnnnnnnnnnnnnnnnnnnnnnnnnnnnnnnnnnnnnnnnnnnnnnntggagagaaaggggaccaaagggagacagaggagagaaaggggagccggggctgcctggtgtttgtagatgtggaagcatcgttctcaaatctgctttttctgttggcatcacaaccagctacccagaagaaaggctaccaatcatatttaacaaggtcctcttcaatgagggagagcattacaaccctgcaacggggaagttcatctgtgctttcccaggaatctattacttttcttatgatatcacattggcgaataagcatctggcaatcgggctggtacacaatgggcagtaccggataaagacctttgatgccaacacaggaaaccatgatgtggcttcagggtccacagtcatttatcttcagccagaagatgaagtctggctggagatcttcttcactgaccagaatggcctcttctcagacccaggttgggcagacagcttgttctccggatttctcctgtacgttgacacagattatctagattccatatcagaagacgacgagctctgatcagcaccgttggcctgaatgtcccaggatccttgtggcagacactttgatttaatctgggcccctgccaggtggaacaagaagaaatgagctccaaaaagactcccactcagattccagagcatttacagttatagcagaatctattaaagcaagttggaaccaccaaactgctgaaatcatgccaaaatgaatttataaaacaataacctcagatatgttttctcaaaagtgatggttcataaatatttaaacaaattaaagacaatgttacaaattttctattaaattccttgagtgctgaaacaaagctggcaatgatattgtctcattaaatttttaaaaaaaaaaaa

SEQ ID NO: 29 - protein, 299 aa, Sus scrofa
  MISC_FEATURE (1)..(299): X = any amino acid
MSAMPRKEPKMFVLLYVTSFAICASGQPRGNQFKGDTYSPRYICSIPGLPGPPGPPGANGSPGPHGRIGLPGRDGRDGRKGEKGEKGAAGLRGKTGPLGLXXXXXXXXXXXXXXXXXXXXXXXXXXXWRERGPKGDRGEKGEPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 30 - DNA, 1240 bp, Sus scrofa
tgcaaaacttcggacgacagcatgagctccaagcttcaaaaggattctcacgcgcggaggagcagtttatttttcatcaagttatcttccgtgtctttttgaagcagcttcgaagcataaacacctatttctgctttaaacttttttaatggtttctaaattcatgggacaaccaaagccagaaagcctcacgctttggaggagcatttgatgtcagcaatgcctagaaaagagccaaagatgtttgtcttgctctatgttacaagttttgccatttgtgcaagtggacaacctcggggtaatcagttcaaaggcgatacctactccccgagatatatctgtagcattcctggcttacctggacctccaggcccccctggagcaaatggttcccctgggccccatggtcggatcggccttccgggacgagatggtagagacggcaggaaaggagaaaaaggtgaaaagggggctgcaggtttgagagctacccagaagaaaggctaccaatcatatttaacaaggtcctcttcaatgagggagagcattacaaccctgcaacggggaagttcatctgtgctttcccaggaatctattacttttcttatgatatcacattggcgaataagcatctggcaatcgggctggtacacaatgggcagtaccggataaagacctttgatgccaacacaggaaaccatgatgtggcttcagggtccacagtcatttatcttcagccagaagatgaagtctggctggagatcttcttcactgaccagaatggcctcttctcagacccaggttgggcagacagcttgttctccggatttctcctgtacgttgacacagattatctagattccatatcagaagacgacgagctctgatcagcaccgttggcctgaatgtcccaggatccttgtggcagacactttgatttaatctgggcccctgccaggtggaacaagaagaaatgagctccaaaaagactcccactcagattccagagcatttacagttatagcagaatctattaaagcaagttggaaccaccaaactgctgaaatcatgccaaaatgaatttataaaacaataacctcagatatgttttctcaaaagtgatggttcataaatatttaaacaaattaaagacaatgttacaaattttctattaaattccttgagtgctgaaacaaagctggcaatgatattgtctcattaaatttttaaaaaaaaaaaa

SEQ ID NO: 31 - protein, 153 aa, Sus scrofa
MSAMPRKEPKMFVLLYVTSFAICASGQPRGNQFKGDTYSPRYICSIPGLPGPPGPPGANGSPGPHGRIGLPGRDGRDGRKGEKGEKGAAGLRATQKKGYQSYLTRSSSMRESITTLQRGSSSVLSQESITFLMISHWRISIWQSGWYTMGSTG

SEQ ID NO: 32 - DNA, 1591 bp, Gallus gallus
cttatttagctgagacaggcagttccaaagctcaatacaagagcatcaagcttcaaataagttttcgtgaagtacattatctgtcatcgaggcgttcagattggctttaacggatttggaaataccaacaattacctgttggcttcaatttacagctcctacggcacgacccgaaagaagggtgttgcttttcctggaaaacattggtgtcatctactcgaatgtaaagaaacctgtggactctgatttgcttctgtgctgtaagtcagcactgaaggaatggacaactccaacaaaaccccccacagatgtgttgtgagagtccagcaagtgaattagaagaaacaactcgactcacaatatctcacctgactcatgtctgaatctcagctaatgcaacgctgattttgaacacctttgtatatcagattcaacgatgtttgtgttgctgtacattacaagttttgccatctacaccagtgaacaacctcttcaaaaccagttcaaaggagagaactaccacaccagatacatctgcagtatccctgggttgccaggtcccgtgggcccccctggagctaacggagccccagggccacatggacgcattggtcttccaggaagagatggtagagatggcaggaaaggagaaaaaggtgaaaaggggagcgcaggtttaagaggaaaaactgggccattaggaccaaccggagagaaaggagaccaaggtcagtctgggaaaaaaggaccaggaggactgattggtgccaagggtgaagtaggtccagctggaccacctggacctaagggagataaaggagaccgaggagagccaggtgcaccaggggtctgtaagtgtgggaagatagtgctgaaatctgccttttctgttggcatcaccaccagctaccctgaggaaagactaccaattgtattcaataaagtcctcttcaatgagggtgaacattacaacccttccacagggaagtttatttgtgccatcccagggatttattatttttcttatgacatcaccttggcaaataagcaccttgcgattgggctggttcacaatgggaagtaccgcataaagacattcgatgcaaacacaggaaaccacgacgtagcttctgggtccactgtgatctaccttcagccagaagatgaagtatggcttgagatcttctacactgaccaaaatggtctcttttcagacccaacatgggcagacagcttgttttctggatttctcttatacgttgatacagattaccttgatgctttatcggacgaagatgagctctgaagcagataatgtcagatcacagcccaaacgacaatccagcacatgactttatctggccgtgtgtaggacacaaagtggacacacatgaataacctgggaattcatttttgctttgacaggaagcaaatctgagttcagattctagaatcaaagctggattctttaccaaacaacagggatatcaaacaggaactataactaacaaaagactgcatcgctccaggacagactcctcatggaagttaatgtttacgatattatttaaacaaatttaagatactgtac

SEQ ID NO: 33 - protein, 289 aa, Gallus gallus
MFVLLYITSFAIYTSEQPLQNQFKGENYHTRYICSIPGLPGPVGPPGANGAPGPHGRIGLPGRDGRDGRKGEKGEKGSAGLRGKTGPLGPTGEKGDQGQSGKKGPGGLIGAKGEVGPAGPPGPKGDKGDRGEPGAPGVCKCGKIVLKSAFSVGITTSYPEERLPIVFNKVLFNEGEHYNPSTGKFICAIPGIYYFSYDITLANKHLAIGLVHNGKYRIKTFDANTGNHDVASGSTVIYLQPEDEVWLEIFYTDQNGLFSDPTWADSLFSGFLLYVDTDYLDALSDEDEL

SEQ ID NO: 34 - DNA, 1196 bp, Gallus gallus
tctagaaggacgcagtcgcaggaggacggctttgtgctacatgacgtgacggcgatctgcagcaaaacatctgctgcaagccgagcggaatcccagcgcctgtcaagacctcgctgaagacattaagcctgggctgtgccatgatttctgctgtcctcctcctctggactgtgccctgcgtggccaaccacatcctcgggggcttcgctaaggcagcgctgcaggaaggcccccagctggcgtgcagcttgccgggcccccctgggcctcccggccctcctggtgcacctgggactccggggacagttggcaggatgggcttcccagggaaagatggcaaggacggcaaggatggggacaaaggcgatcacggtgatgaaggtccacagggcagaacaggaaacccaggcaagccaggaccgaagggcaaagcaggagccattggcaaggcaggcccacgaggaccaaagggtttaaagggtaatcctggaaaaaacggagcaccaggaaagaaaggacccaaagggaacaaaggtgagactgggatgccaggaccctgcacctgcaatgccaacaaagccaaatctgccttctccgtagctgtctctaagagctacccaagggaaaggctccccatcaaatttgacaggatcctgatgaacgagggaggacattacaatgcttccagtgggaaattcgtatgcagcatcccaggtatttactacttcacttatgatatcactttggccaacaaacatctggccattggcttggtccacaacgggcagtaccggatcaagacttttgatgccaacactgggaaccatgacgttgcctctggatcaaccattctctctctaaagcaagaggatgaggtgtggctgcagataagactcactttttaaagcagagagtgtgaaccttcactccagaaacaggtgtatcaacagaacctgacattaactgtgcaagcccaatgcctgcagatagactctgtcatactgacgtgctttgctttgaggtatggtatgtcaaatattacacatgatccgatcttaacatagtagttctaaactgtattaagaagaaaaacaaggcagaaaaggccagaggtgagctttttggtcttgctatgatgagctttgcatttttccccatcccaaccagcagccatacagggatacgctcccaaacgg

SEQ ID NO: 35 - protein, 254 aa, Gallus gallus
MISAVLLLWTVPCVANHILGGFAKAALQEGPQLACSLPGPPGPPGPPGAPGTPGTVGRMGFPGKDGKDGKDGDKGDHGDEGPQGRTGNPGKPGPKGKAGAIGKAGPRGPKGLKGNPGKNGAPGKKGPKGNKGETGMPGPCTCNANKAKSAFSVAVSKSYPRERLPIKFDRILMNEGGHYNASSGKFVCSIPGIYYFTYDITLANKHLAIGLVHNGQYRIKTFDANTGNHDVASGSTILSLKQEDEVWLQIRLTF

SEQ ID NO: 36 - DNA, 1652 bp, Gallus gallus
tctagaaggacgcagtcgcaggaggacggctttgtgctacatgacgtgacggcgatctgcagcaaaacatctgctgcaagccgagcggaatcccagcgcctgtcaagacctcgctgaagacattaagcctgggctgtgccatgatttctgctgtcctcctcctctggactgtgccctgcgtggccaaccacatcctcgggggcttcgctaaggcagcgctgcaggaaggcccccagctggcgtgcagcttgccgggcccccctgggcctcccggccctcctggtgcacctgggactccggggacagttggcaggatgggcttcccagggaaagatggcaaggacggcaaggatggggacaaaggcgatcacggtgatgaaggtccacagggcagaacaggaaacccaggcaagccaggaccgaagggcaaagcaggagccattggcaaggcaggcccacgaggaccaaagggtttaaagggtaatcctggaaaaaacggagcaccaggaaagaaaggacccaaagggaacaaaggtgagactgggatgccaggaccctgcacctgcaatgccaacaaagccaaatctgccttctccgtagctgtctctaagagctacccaagggaaaggctccccatcaaatttgacaggatcctgatgaacgagggaggacattacaatgcttccagtgggaaattcgtatgcagcatcccaggtatttactacttcacttatgatatcactttggccaacaaacatctggccattggcttggtccacaacgggcagtaccggatcaagacttttgatgccaacactgggaaccatgacgttgcctctggatcaaccattctctctctaaagcaagaggatgaggtgtggctgcagataagactcactttttaaagagagagtgtgaaccttcactccagaaacaggtgtatcaactcccactcctccatcccttccagcttttgctggaaggaaatactcatggtgctgctgaggtgctcctcacaggcttaatgctgcagatagactctgtcatactgacgtgctttgcttttttgtttttgtatgtcaaatattacacatgatccgatcttaacatagtagttctaaactgtattaagaagaaaaacaaggcagaaaaggccagaggtgagctttttggtcttgctatgatgagctttgcatttttccccatcccaaccagcagccatacagggatacgctcccaaacggagcattattgtcacagcaaagaggtctcctgctgtatggctgctcagctgcttgccttacccccgtgtgtgcagcctctctctaccaggagtctgtctgcagtcagagggaaggagtcttcctcaatatctgacacatcttcctggttctcacagtcatatttcttgtctcctctactgcaacacaaaaactcacatcgatttccagttcctctcatctgcacattgagacattgaggagatagcccagaagaacaaattaagatatatatacatatatattaaccttacttctacaagtcattatatctgcatgattttgtgacattacttggtgtcatatcttacaggaaatcttttccaaatactttatatttaaattaagctgaacaaaactaccgggcaaaaa

SEQ ID NO: 37 - protein, 254 aa, Gallus gallus; identical to SEQ ID NO: 35

SEQ ID NO: 38 - DNA, 24 bp, Artificial
  Primer sequence
tcctgcagcaccccaccatctaag

SEQ ID NO: 39 - DNA, 31 bp, Artificial
  Primer sequence
accccagattcaatcagagtgtttaccacag

SEQ ID NO: 40 - DNA, 30 bp, Artificial
  Primer sequence
ccggctagccaaccccggggtaatcagttg

SEQ ID NO: 41 - DNA, 38 bp, Artificial
  Primer sequence
tgctctagactcaattcatcatcttctgatatggaatc

SEQ ID NO: 42 - DNA, 26 bp, Artificial
  Primer sequence
ccggctagccctggcttgcctggacc

SEQ ID NO: 43 - DNA, 33 bp, Artificial
  Primer sequence
ccggctagcggaactgcaggtttgagaggtaag

SEQ ID NO: 44 - DNA, 33 bp, Artificial
  Primer sequence
ccggctagcggagtttgcagatgtggaagcatc

SEQ ID NO: 45 - DNA, 33 bp, Artificial
  Primer sequence
ccggctagcgccttttctgttggcatcacaacc

SEQ ID NO: 46 - DNA, 38 bp, Artificial
  Primer sequence
gattccatatcagaagatgatgaattgagtctagagca

SEQ ID NO: 47 - DNA, 24 bp, Artificial
  Primer sequence
atgcaaccccggggtaatcagttg

SEQ ID NO: 48 - DNA, 19 bp, Artificial
  Primer sequence
caattcatcatcttctgat

SEQ ID NO: 49 - DNA, 24 bp, Artificial
  Primer sequence
atgcctggcttgcctggacctcca

SEQ ID NO: 50 - DNA, 24 bp, Artificial
  Primer sequence
atgggaactgcaggtttgagaggt

SEQ ID NO: 51 - DNA, 24 bp, Artificial
  Primer sequence
atgggagtttgcagatgtggaagc

SEQ ID NO: 52 - DNA, 24 bp, Artificial
  Primer sequence
atggccttttctgttggcatcaca

SEQ ID NO: 53 - DNA, 870 bp, Homo sapiens
atgtttgtcttgctgtatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctccaggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 54 - protein, 274 aa, Homo sapiens
MQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 55 - DNA, 819 bp, Homo sapiens
caaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctccaggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattg

SEQ ID NO: 56 - protein, 274 aa, Homo sapiens; identical to SEQ ID NO: 54

SEQ ID NO: 57 - DNA, 759 bp, Homo sapiens
cctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctccaggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattg

SEQ ID NO: 58 - protein, 253 aa, Homo sapiens
PGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 59 - DNA, 639 bp, Homo sapiens
ggaactgcaggtttgagaggtaagactggaccgctaggtcttgccggtgagaaaggggaccaaggagagactgggaagaaaggacccataggaccagagggagagaaaggagaagtaggtccaattggtcctccaggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattg

SEQ ID NO: 60 - protein, 213 aa, Homo sapiens
GTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 61 - DNA, 459 bp, Homo sapiens
ggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattg

SEQ ID NO: 62 - protein, 153 aa, Homo sapiens
GVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 63 - DNA, 423 bp, Homo sapiens
gccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattg

SEQ ID NO: 64 - protein, 141 aa, Homo sapiens
AFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 65 - DNA, 870 bp, Homo sapiens; identical to SEQ ID NO: 5

SEQ ID NO: 66 - DNA, 765 bp, Homo sapiens
attcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 67 - protein, 274 aa, Homo sapiens; identical to SEQ ID NO: 54

SEQ ID NO: 68 - DNA, 765 bp, Homo sapiens
atgcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 69 - protein, 254 aa, Homo sapiens
MPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 70 - DNA, 645 bp, Homo sapiens
atgggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 71 - protein, 214 aa, Homo sapiens
MGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 72 - DNA, 465 bp, Homo sapiens
atgggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 73 - protein, 154 aa, Homo sapiens
MGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 74 - DNA, 429 bp, Homo sapiens
atggccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 75 - protein, 142 aa, Homo sapiens
MAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

(19) United States
(12) Patent Application Publication
(10) Pub. No.: US 2005/0180949 A1
(43) Pub. Date: Aug. 18, 2005

(54) hC1Q/TNF7 and uses thereof

(75) Inventors: Peter C.R. Emtage, Sunnyvale, CA (US); Shouchun Liu, Burlingame, CA (US); Kerri Thai, Milpitas, CA (US)

Correspondence Address: NUVELO, INC, 675 ALMANOR AVE., SUNNYVALE, CA, 94085, US

(73) Assignee: NUVELO, Inc., Sunnyvale, CA (US)

(21) Appl. No.: 11/057,027

(22) Filed: Feb. 11, 2005

Related U.S. Application Data
(60) Provisional application No. 60/544,806, filed on Feb. 13, 2004.

(51) Int. Cl. A61K 38/19; A61K 48/00; C12Q 1/68

(52) U.S. Cl. 424/85.1; 514/44; 435/6

(57) ABSTRACT

The invention relates to pharmaceutical compositions comprising hC1Q/TNF7 polynucleotides and polypeptides. The invention further relates to the therapeutic use of hC1Q/TNF7 to prevent or treat conditions or disorders associated with wasting disorders, such as cachexia, or diseases associated with impaired growth.

This application claims the benefit of priority to U.S. Provisional Patent Application Ser. No. 60/544,806 filed Feb. 13, 2004 entitled “NUVO9452 and Uses Thereof,” Attorney Docket No. NUVO-15. This and all other patent and patent applications are herein incorporated by reference in their entirety.

### 1. BACKGROUND

1.1 Field of the Invention

The present invention relates generally to compositions that comprise hC1Q/TNF7 polypeptides and polynucleotides, and methods for using the same.

1.2 Sequence Listing

The sequences of the polynucleotides and polypeptides of the invention are listed in the Sequence Listing and are submitted on a compact disc containing the file labeled “NUVO-15CP.txt”—104 KB 106,496 bytes) which was created on an IBM PC, Windows 2000 operating system on Feb. 10, 2005 at 10:20:58 AM. The Sequence Listing entitled “NUVO-15CP.txt” is herein incorporated by reference in its entirety. A computer readable format (“CRF”) and two duplicate copies (“Copy 1/2” and “Copy 2/2”) of the Sequence Listing “NUVO-15CP.txt” are submitted herein. Applicants hereby state that the content of the CRF and Copies 1/2 and 2/2 of the Sequence Listing, submitted in accordance with 37 CFR §1.821(c) and (e), respectively, are the same

1.3 Background

The cachexia, or wasting, syndrome is characterized by a marked weight loss, anorexia (loss of appetite), asthenia (weakness) and anemia (drop in hemoglobin level). Cachexia is often associated with cancer and the growth and/or metastasis of malignancies and is responsible for 22% of the deaths from cancer (reviewed in Argilés et al., Drug Disc. Today 8:838-844 (2003) herein incorporated by reference in its entirety). However, it is associated with many chronic or end-stage diseases, including acquired immunodeficiency syndrome (AIDS), chronic obstructive pulmonary disease (COPD), advanced kidney disease, advanced cardiovascular disease, severe infection, tuberculosis, Crohn's disease, and autoimmune diseases including rheumatoid arthritis and systemic lupus erythematosus (SLE). In addition, treatments such as chemotherapy and radiation can induce a cachexia-like wasting. The wasting arises in part from reduced food intake resulting in anorexia and malnutrition. However, cachexia is more than lack of caloric intake, it is a complex metabolic state with progressive depletion of host reserves of adipose tissue (fatty body mass) and skeletal muscle and bone (collectively known as lean body mass). The metabolic changes result in altered carbohydrate, protein and lipid metabolism (Fearon and Moses, Int. J. Cardiol. 85:73-81 (2002); Costelli and Baccino, Curr. Opin. Clin. Nutr. Metab. Care 3:177-181 (2000) both of which are herein incorporated by reference in their entirety). Cachexia represents the clinical consequence of a chronic, systematic inflammatory response, presumably due to the invading tumor or disease state, with high hepatic synthesis of acute-phase proteins, such as tumor necrosis factor alpha (TNFα), resulting in depletion of amino acids.

A well-established prophylaxis or therapy for cachexia is unavailable. The lack of nutrients alone cannot explain the metabolic changes seen in cachexia. In clinical trials, nutritional supplements, dietary counseling, and appetite-stimulating drugs failed to increase body weight, especially lean body mass. Only a few limited treatment options are available for cachetic patients. Corticosteroids have been shown to have a temporary stimulation of body mass gain, albeit primarily in fatty tissue and fluid retention. However, many corticosteroids interfere with cancer chemotherapeutic agents and after 3-4 weeks of treatment, begin to interfere with synthesis of muscle protein. Another treatment with limited success utilizes the COX-2 inhibitors which have been shown to reduce the weight loss in cachetic patients. Currently, the appetite stimulant megastrol acetate is widely used to treat cachexia although the weight gain is temporary and mainly comes as fatty tissue with little improvement in protein balance.

Thus, there is a need to find agents that may be used prophylactically or therapeutically to reverse weight loss, specifically reverse the loss of lean body mass, and to advance current therapies for treating other disorders associated with weight loss, such as anorexia and bulimia, and other diseases with growth or metabolic deficiencies.

To this end, Applicants have discovered an agent which stimulates the growth of both fatty and lean body mass and which may be useful for treating conditions in which an increase of body mass is desired.

### 2. SUMMARY OF THE INVENTION

The present invention is based on the discovery that hC1Q/TNF7 increases body mass, both fatty and lean body mass. Thus, compositions comprising hC1Q/TNF7, fragments or analogs thereof, may be used for the treatment of conditions where an increase in body mass is required, such as for the treatment of wasting disorders, such as cachexia, eating disorders, or diseases with a growth deficiency, such as genetic dwarfism or the growth retardation associated with pediatric Crohn's disease.

Accordingly, in one embodiment, the invention is directed to a composition comprising a therapeutically effective amount of a hC1Q/TNF7 polypeptide and a pharmaceutically acceptable carrier.

The compositions of the present invention include isolated polynucleotides encoding hC1Q/TNF7 polypeptides, including recombinant DNA molecules, and cloned genes or degenerate variants thereof, especially naturally occurring variants such as allelic variants. Specifically, the polynucleotides of the present invention are based on a hC1Q/TNF7 polynucleotide (SEQ ID NO: 1).

The compositions of the present invention also include vectors such as expression vectors containing the polynucleotides of the invention, cells genetically engineered to contain such polynucleotides and cells genetically engineered to express such polynucleotides.

The compositions of the invention comprise isolated polynucleotides that include, but are not limited to, a hC1Q/TNF7 polynucleotide, a fragment, or variant thereof; a polynucleotide comprising the full length protein coding sequence of the SEQ ID NO: 3 or 5 (for example, SEQ ID NO: 4); polynucleotides comprising the V5-His-tagged protein sequences of SEQ ID NO: 10, 54, 56, 58, 60, 62, 64, 67, 69, 71, 73, or 75 (for example SEQ ID NO: 9, 53, 55, 57, 59, 61, 63, 65-66, 68, 70, 72, or 74); a polynucleotide comprising the nucleotide sequence of the mature protein coding sequence of SEQ ID NO: 7 (for example SEQ ID NO: 8). The polynucleotide compositions of the present invention also include, but are not limited to, a polynucleotide that hybridizes under stringent hybridization conditions to (a) the complement of any of the nucleotide sequences set forth in SEQ ID NO: 1-3, 5, 7, 9, 11-14, 38-53, 55, 57, 59, 61, 63, 65-66, 68, 70, 72, or 74; (b) a nucleotide sequence encoding any of SEQ ID NO: 1-3, 5, 7, 9, 11-14, 38-53, 55, 57, 59, 61, 63, 65-66, 68, 70, 72, or 74; a polynucleotide which is an allelic variant of any polynucleotides recited above having at least 70% polynucleotide sequence identity to the polynucleotides; a polynucleotide which encodes a species homolog (e.g. ortholog) of any of the peptides recited above, for example SEQ ID NO: 16-37; or a polynucleotide that encodes a polypeptide comprising a specific domain or truncation of the polypeptide of SEQ ID NO: 4, 6, 8, 10, 54, 56, 58, 60, 62, 64, 67, 69, 71, 73, or 75.

This invention further provides cloning or expression vectors comprising at least a fragment of the polynucleotides set forth above and host cells or organisms transformed with these expression vectors. Useful vectors include plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The pharmaceutical compositions of the present invention include polypeptides comprising, but not limited to, an isolated polypeptide selected from the group comprising the amino acid sequence of SEQ ID NO: 4, 6, 8, 10, 54, 56, 58, 60, 62, 64, 67, 69, 71, 73, or 75. Polypeptides of the invention also include polypeptides with biological activity that are encoded by (a) any of the polynucleotides having a nucleotide sequence set forth in the SEQ ID NO: 1-3, 5, 7, 9, 11-14, 38-53, 55, 57, 59, 61, 63, 65-66, 68, 70, 72, or 74 above; or (b) polynucleotides that hybridize to the complement of the polynucleotides of (a) under stringent hybridization conditions. Biologically or immunologically active analogs of any of the protein sequences listed as SEQ ID NO: 4, 6, 8, 10, 54, 56, 58, 60, 62, 64, 67, 69, 71, 73, or 75, and substantial equivalents thereof that retain biological are also contemplated. The polypeptides of the invention may be wholly or partially chemically synthesized but are preferably produced by recombinant means using the genetically engineered cells (e.g. host cells) of the invention.

The invention also relates to methods for producing a hC1Q/TNF7 polypeptide comprising culturing host cells comprising an expression vector containing at least a fragment of a hC1Q/TNF7 polynucleotide encoding the hC1Q/TNF7 polypeptide of the invention in a suitable culture medium under conditions permitting expression of the desired polypeptide, and purifying the protein or peptide from the culture or from the host cells. Preferred embodiments include those in which the protein produced by such a process is a mature form of the protein.

The polypeptides according to the invention can be used in a variety of conventional procedures and methods that are currently applied to other proteins. For example, a polypeptide of the invention can be used to generate an antibody that specifically binds the polypeptide. Such antibodies, particularly monoclonal antibodies, are useful for detecting or quantifying the polypeptide in tissue.

In further embodiments, the subject invention is directed to a method of stimulating growth of fatty and lean body mass. The method comprises administering a subject in need of hC1Q/TNF7 therapy with a composition that includes a therapeutically effective amount of a hC1Q/TNF7 polypeptide, fragment or analog thereof, and a pharmaceutically acceptable carrier. Specifically, a subject in need of increased body mass will be administered therapeutically-effective or prophylactically-effective amounts of hC1Q/TNF7 protein, fragments or analogs thereof.

Methods are also provided for preventing, treating, or ameliorating a medical condition which comprises the step of administering to a mammalian subject a therapeutically effective amount of a composition comprising a peptide of the present invention and a pharmaceutically acceptable carrier.

In particular, the hC1Q/TNF7 polypeptides of the invention can be used to increase body mass. hC1Q/TNF7 can act as an appetite stimulant (i.e. increases caloric intake), a metabolic regulator (i.e. lowering the metabolic rate or increasing the efficiency of metabolism for any of carbohydrates, lipids, and protein), or an inducer of tissue growth (i.e. a growth factor-like molecule). Thus, the hC1Q/TNF7 polypeptides and polynucleotides of the invention can be used in the treatment of wasting disorders, such as disease- or treatment-induced cachexia, eating disorders, such as anorexia, and growth disorders, such as genetic dwarfism. They can also be used in the treatment of diseases, and other conditions including growth hormone deficiencies, symptoms of ageing, long-term convalescence and therapy for coma patients.

The methods of the invention also provide methods for the treatment of disorders as recited herein which comprise the administration of a therapeutically effective amount of a composition comprising a polynucleotide or polypeptide of the invention and a pharmaceutically acceptable carrier to a mammalian subject exhibiting symptoms or tendencies related to disorders as recited herein. In addition, the invention encompasses methods for treating diseases or disorders as recited herein comprising the step of administering a composition comprising compounds and other substances that modulate the overall activity of the target gene products and a pharmaceutically acceptable carrier. Compounds and other substances can effect such modulation either on the level of target gene/protein expression or target protein activity. Specifically, methods are provided for preventing, treating or ameliorating a medical condition, including wasting disorders, which comprises administering to a mammalian subject, including but not limited to humans, a therapeutically effective amount of a composition comprising a polypeptide of the invention or a therapeutically effective amount of a composition comprising a binding partner of hC1Q/TNF7 polypeptides of the invention. The mechanics of the particular condition or pathology will dictate whether the polypeptides of the invention or binding partners of these would be beneficial to the individual in need of treatment.

The invention further provides methods for manufacturing medicaments useful in the above-described methods.

The present invention further relates to methods for detecting the presence of the polynucleotides or polypeptides of the invention in a sample (e.g., tissue or sample). Such methods can, for example, be utilized as part of prognostic and diagnostic evaluation of disorders as recited herein and for the identification of subjects exhibiting a predisposition to such conditions.

The invention provides a method for detecting a polypeptide of the invention in a sample comprising contacting the sample with a compound that binds to and forms a complex with the polypeptide under conditions and for a period sufficient to form the complex and detecting formation of the complex, so that if a complex is formed, the polypeptide is detected.

The invention also provides kits comprising polynucleotide probes and/or monoclonal antibodies, and optionally quantitative standards, for carrying out methods of the invention. Furthermore, the invention provides methods for evaluating the efficacy of drugs, and monitoring the progress of patients, involved in clinical trials for the treatment of disorders as recited above.

The invention also provides methods for the identification of compounds that modulate (i.e., increase or decrease) the expression or activity of the polynucleotides and/or polypeptides of the invention. Such methods can be utilized, for example, for the identification of compounds that can enhance the therapeutic activity of the hC1Q/TNF7 polypeptides, and ameliorate symptoms of disorders as recited herein. Such methods can include, but are not limited to, assays for identifying compounds and other substances that interact with (e.g., bind to) the polypeptides of the invention.

The invention provides a method for identifying a compound that binds to the polypeptide of the present invention comprising contacting the compound with the polypeptide under conditions and for a time sufficient to form a polypeptide/compound complex and detecting the complex, so that if the polypeptide/compound complex is detected, a compound that binds to the polypeptide is identified.

Also provided is a method for identifying a compound that binds to the polypeptide comprising contacting the compound with the polypeptide in a cell for a time sufficient to form a polypeptide/compound complex wherein the complex drives expression of a reporter gene sequence in the cell and detecting the complex by detecting reporter gene sequence expression so that if the polypeptide/compound complex is detected a compound that binds to the polypeptide is identified.

Another embodiment of the invention provides a cell-based method to identify the in vivo biological phenotype and activity of a target protein, including but not limited to hC1Q/TNF7, in which an expression vector encoding the target gene is transfected into mammalian host cells which are subsequently administered to a subject animal, i.e. Nude mice, and the in vivo phenotype or activity is determined.

In a related embodiment, the invention is directed to use of a vector comprising a gene encoding a hC1Q/TNF7 polypeptide operably associated with an expression control sequence that provides for expression of the hC1Q/TNF7 polypeptide in the manufacture of a medicament for treating disorders as recited herein. More particularly, the invention provides for use of a hC1Q/TNF7 vector of the invention, e.g., as set out below, in the manufacture of a medicament for treating wasting disorders, and other disorders requiring hC1Q/TNF7 therapy.

In another aspect the invention concerns a method of screening drug candidates for the treatment of a disease or disorder recited herein comprising (a) administering a drug candidate to a mouse that expresses a hC1Q/TNF7 polypeptide, and develops increased body mass, and (b) evaluating the effect of the candidate drug on the increased growth. The drug candidates may modulate (i.e. increase or decrease) the expression or activity of the polynucleotides and/or polypeptides of the invention.

Additional aspects and advantages of the invention will be apparent to those skilled in the art upon consideration of the following description, which details the practice of the invention.

### 3. BRIEF DESCRIPTION OF THE DRAWINGS

For all figures, amino acids are abbreviated as follows: A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine.

For all figures, nucleic acids are abbreviated as follows: A=Adenine, C=Cytosine, G=Guanine, T=Thymine.

FIG. 1 depicts (A) the DNA sequence (SEQ ID NO: 3) and (B) the corresponding amino acid sequence (SEQ ID NO: 4) for the full-length human C1Q/TNF7 (hC1Q/TNF7) of the invention. SEQ ID NO: 2 includes the 5-prime and 3-prime untranslated regions in conjunction with the open reading frame (underlined).

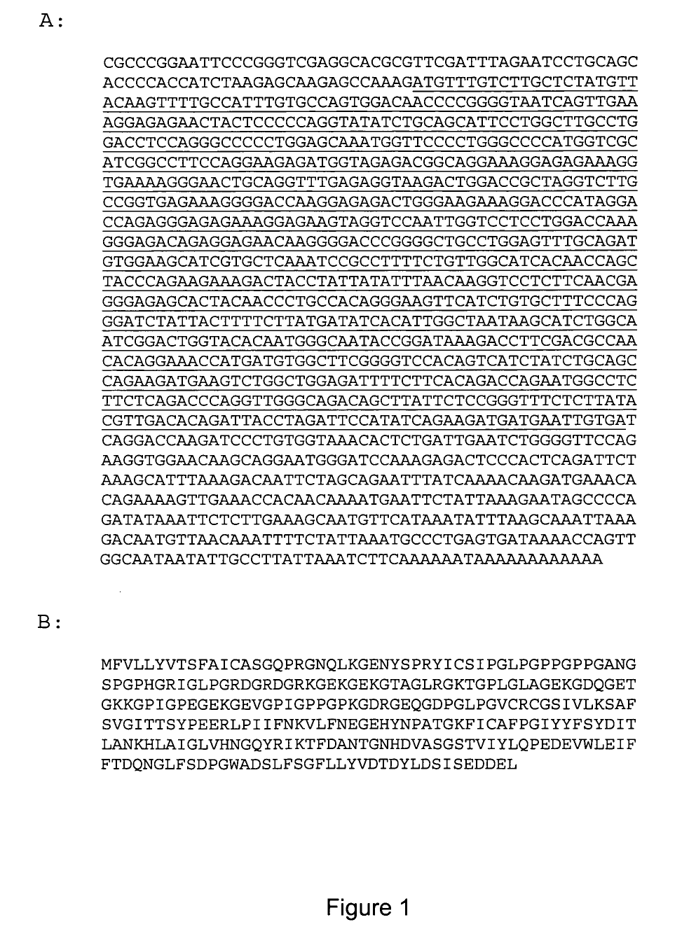

FIG. 2 depicts a BLASTP amino acid sequence alignment of SEQ ID NO: 4 with human C1qTNF-7, gi:13994280 (SEQ ID NO: 15) showing 100% identity over 289 amino acid residues of SEQ ID NO: 15.

FIG. 3 shows the three-dimensional (3D) structural similarity between hC1Q/TNF7 and adiponectin. The crystal structure of adiponectin (accession number 1C28, RCSB Protein Data Bank (Berman et al., Nucl. Acids Res. 28:235-242 (2000) herein incorporated by reference in its entirety) and structural model of hC1Q/TNF7 based on the structural model of adiponectin is shown. Both structures follow a 10 β-strand jelly roll folding topology (Shapiro and Scherer, Curr. Biol. 8:335-338 (1998), herein incorporated by reference in its entirety). The eight amino acid residues that are conserved between both proteins are labeled.

FIG. 4 depicts the expression of hC1Q/TNF7 mRNA in human tissues.

FIG. 5 shows the gross pathology of mice injected with hC1Q/TNF7-expressing HEK293 cells: (A) GFP control, (B) hC1Q/TNF7.

FIG. 6 shows a graphic comparison of (A) total body weight of hC1Q/TNF7 mice compared to GFP controls, and (B) body length of hC1Q/TNF7 mice compared to GFP controls.

FIG. 7 depicts a schematic presentation of full-length and nested truncations of recombinant hC1Q/TNF7 proteins. The numbers indicate the amino acid positions of the mature hC1Q/TNF7 protein.

FIG. 8 shows a Western blot visualized using phoso-Erk1/2 and Erk1/2 antibodies to determine the activity of hC1Q/TNF7 on Erk1/2 phosphorylation. C2C12 cells were treated for 30 min as described below using the following recombinant proteins at a concentration of 0.5 μg/ml: 1. PBS; 2. full-length hC1Q/TNF7 (HEK-293-expressed); 3. hC1Q/TNF7-F1 (HEK-293-expressed); 4. hC1Q/TNF7-F2 (bacteria-expressed); and 5. hC1Q/TNF7-F4 (bacteria-expressed).

FIG. 9 shows a Western blot visualized using an HRP-conjugated anti-V5 antibody to determine the oligomerization state of hC1Q/TNF7 proteins. Purified recombinant full-length hC1Q/TNF7 protein expressed in HEK-293 cells was treated as follows: N=non-reduced only; N+H=non-reduced plus heating at 100° C. for 10 min; R=reduced only; and R+H=reduced plus heating at 100° C. for 10 min.

FIG. 10 depicts a Western blot visualized using an HRP-conjugated anti-V5 antibody to determine if hC1Q/TNF7 is glycosylated. Purified mammalian hC1Q/TNF7-F1 protein was treated under denaturing conditions as follows: 1. Control; 2. PNGase F (peptide: N-Glycosidase F); 3. PNGase F+Sialidase A; and 4. PNGase F+Sialidase A+O-Glucanase.

FIG. 11 depicts a Western blot visualized using an HRP-conjugated anti-V5 antibody to analyze hC1Q/TNF7 stability. Purified hC1Q/TNF7-F1 protein was stored for the time and temperature indicated as follows: 1: Day 0, no treatment; 2, 3, 4, and 5: protein was stored at 4° C. for 1, 3, 4, or 5 days, respectively; 6, 7, 8, and 9: protein was stored at 37° C. for 1, 3, 4, or 5 days, respectively.

FIG. 12 demonstrates the induction of AMPK phosphorylation by hC1Q/TNF7. All samples (clarified cell lysates) were visualized using anti-phospho-AMPK or anti-AMPK antibodies. A. Purified hC1Q/TNF7-F1 protein was treated as indicated: C=untreated control; H-I=heat-inactivated; Ni-D=Ni²⁺-bead depleted; and V5-D=anti-V5 antibody depleted. The remaining protein in each sample was detected using anti-V5 antibody. B. C2C12 myoblasts were treated with 0.5 μg/ml purified hC1Q/TNF7-F1 protein for different times as indicated. C. C2C12 myoblasts were treated with different concentrations of hC1Q/TNF7-F1 protein as indicated for 30 min.

FIG. 13 demonstrates the induction of ACC phosphorylation by hC1Q/TNF7-F1. A. C2C12 myoblasts were incubated with recombinant hC1Q/TNF7-F1 protein at the indicated concentrations for 30 min. Western blot analysis was performed using anti-phospho-ACC antibodies. The cell lysate was also blotted with anti-β-actin antibodies to estimate the loading of each sample. B. Cell lysate from hC1Q/TNF7-F1 and PBS-treated C2C12 myoblasts was analyzed by Western blot as in A. The quantity of normalized phospho-ACC at each time point was estimated by scanning densitometry using the Image program (National Institutes of Health) and expressed as a percentage of maximum phospho-ACC content at 30 min after treatment.

FIG. 14 demonstrates Erk1/2 phosphorylation by hC1Q/TNF7-F1 protein. A. C2C12 myoblasts were incubated with hC1Q/TNF7-F1 protein at the concentrations indicated. Erk1/2 phosphorylation in each sample was determined by Western blot analysis using phospho-Erk1/2 specific antibodies. Equal loading was confirmed by Western blot analysis using anti-Erk1/2 antibodies. B. C2C12 myoblasts were incubated with PBS, hC1Q/TNF7-F1 (1 μg/ml), and gAd (2 μg/ml) for indicated times. Western blot analysis was performed as described above. C. The quantity of normalized phospho-Erk1/2 at each time point was estimated by scanning densitometry using the Image program and expressed as a percentage of maximum phopho-Erk1/2 content at 30 min after treatment.

FIG. 15 demonstrates increased glucose uptake by recombinant hC1Q/TNF7 proteins. A. C2C12 myoblasts were treated with PBS, human insulin, hC1Q/TNF7-F1 (mammalian-expressed) and hC1Q/TNF7-F4 (bacteria-expressed) at the indicated concentrations and incubated with 2-Deoxy-D-[1-³H]2-glucose. Uptake of 2-Deoxy-D-[1-³H]2-glucose in C2C12 cells was measured by liquid scintillation counting. B. C2C12 myoblasts were treated with PBS, human insulin alone (Ins, 1 μg/ml), hC1Q/TNF7-F1 alone (F1, 1 μg/ml), hC1Q/TNF7-F4 alone (F4, 1 μg/ml), human insulin+hC1Q/TNF7-F1 (Ins+F1, each at 1 μg/ml), and human insulin+hC1Q/TNF7-F4 (Ins+F4, each at 1 μg/ml). The C2C12 uptake of 2-Deoxy-D-[1-³H]2-glucose was counted using a liquid scintillation counter.

FIG. 16 demonstrates increased fatty acid oxidation by recombinant hC1Q/TNF7 proteins. C2C12 myoblasts were treated with PBS, gAd (2 μg/ml), hC1Q/TNF7-F1 (1 μg/ml), and hC1Q/TNF7-F4 (1 μg/ml) and incubated with [1-¹⁴C]-Palmitate. The formation of ¹⁴CO₂ was measured using a liquid scintillation counter. The data represent the mean±S.D. of the triplicates. Each experiment was repeated at least twice.

### 4. DETAILED DESCRIPTION OF THE INVENTION

A. Protein Characteristics

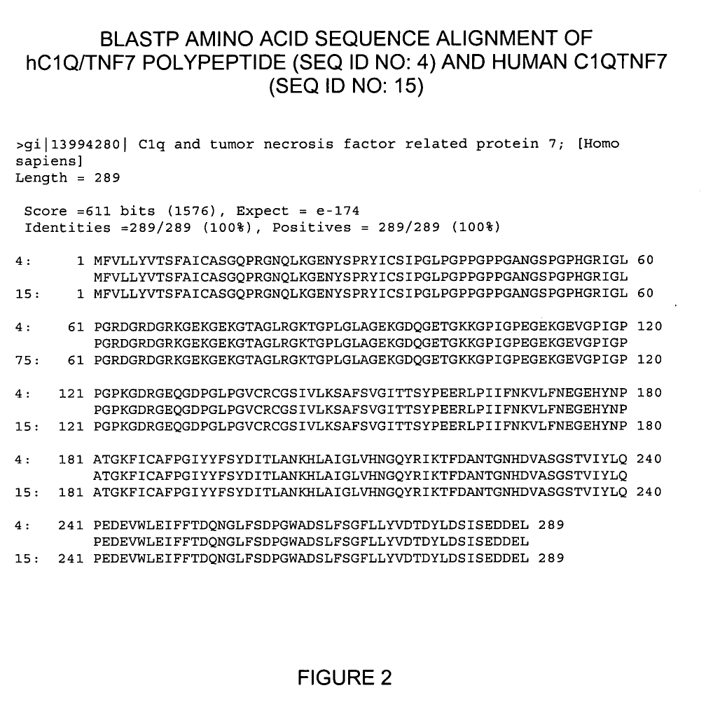

The hC1Q/TNF7 polypeptide of SEQ ID NO: 4 is an approximately 289 amino acid protein with a predicted molecular mass of approximately 31.7 kDa unglycosylated. The initial methionine starts at position 80 of SEQ ID NO: 3 and the putative stop codon begins at position 947 of SEQ ID NO: 3. Protein database searches with the BLAST algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 4 shares 100% identity with human C1qTNF-7 (gi:13994280, SEQ ID NO: 15) over 289 amino acids of SEQ ID NO: 15 (see FIG. 2).

A predicted approximately sixteen-residue signal peptide (SEQ ID NO: 6) is encoded from residue 1 to residue 16 of SEQ ID NO: 4. The extracellular portion is useful on its own. The signal peptide region was predicted using the Neural Network Signal P VI.I program (Nielsen et al., Int. J. Neural Syst. 8:581-599 (1997)), incorporated herein by reference). One of skill in the art will recognize that the actual cleavage site may be different than that predicted by the computer program. SEQ ID NO: 8 is the HC1Q/TNF7 polypeptide of SEQ ID NO: 4 that lacks the signal peptide (SEQ ID NO: 6).

In addition, the amino acid sequence of hC1Q/TNF7 contains two consensus sequences for potential N-glycosylation sites (N-X-S/T), wherein N=Asparagine, X=any amino acid, S=Serine, T=Threonine. These sites begin at residues 25 and 49 of SEQ ID NO: 4

Using the Pfam software program (Sonnhammer et al., Nucl. Acids Res. 26:320-322 (1998) herein incorporated by reference in its entirety), the hC1Q/TNF7 polypeptide of SEQ ID NO: 4 was determined to have structural homology to C1q and collagen domains (see Table 1). The results describe e-value, score, model, description, and amino acid position of the domain in the full-length protein.

TABLE 1

| | | | | Amino acid |
|---|---|---|---|---|
| e-value | Score | Model | Description | position |
| 1.3e−05 | 25.8 | Collagen | Collagen triple helix repeat | 37-73 |
| | | | (20 copies) | |
| 2e−11 | 47.0 | Collagen | Collagen triple helix repeat | 77-136 |
| | | | (20 copies) | |
| 1.3e−40 | 148.4 | C1q | C1q domain | 149-273 |

Using the eMATRIX software package (Stanford University, Stanford, Calif.) (Wu et al., J. Comp. Biol. 6:219-235 (1999) herein incorporated by reference in its entirety), the HC1Q/TNF7 polypeptide of SEQ ID NO: 4 was determined to have the following eMATRIX domain hits with e-values less than 1e-07 (see Table 2). The results describe: Accession number, name, and the position of the domain in the full-length protein.

TABLE 2

| Accession | | Amino acid |
|---|---|---|
| number | Name | position |
| IPB000885B | Fibrillar collagen C-terminal domain | 3-161 |
| IPB001442A | C-terminal tandem repeated domain in type | 10-164 |
| | 4 procollagen | |
| IPB001073B | Complement C1q protein | 31-275 |
| PR00007A | Complement C1q domain signature I | 158-184 |
| PR00007B | Complement C1q domain signature II | 185-204 |
| PR00007C | Complement C1q domain signature III | 229-250 |
| PR00007D | Complement C1q domain signature IV | 264-274 |

B. hC1Q/TNF7 Activities

hC1Q/TNF7 is a C1q domain-containing protein that also shares homology to TNFα. C1q protein is the first component of the classical complement pathway and binds to antigen-bound antibodies. Many non-component proteins have been identified that contain C1q domains. Most have a similar structure comprising a leading signal peptide, followed by a collagen-like domain and a C-terminal C1q domain (reviewed in Kishore and Reid, Immunopharmacology 42:15-21 (1999) herein incorporated by reference in its entirety). Both the structure and sequence of the C1q domains are conserved; however their functions are not. There are many C1q proteins that are not involved in the complement pathway, including human type X collagen (Ninomiya et al. J. Biol. Chem. 274:16773 (1999) herein incorporated by reference in its entirety) and adiponectin (Scherer et al. J. Biol. Chem. 270:26746 (1995) herein incorporated by reference in its entirety). Type X collagen (COL10A1) is specifically expressed by hypertrophic chondrocytes during bone development (Thomas et al., Biochem. Soc. Trans. 19:804-808 (1991) herein incorporated by reference in its entirety).

Adiponectin (also known as Acrp30 and AdipoQ) is an anti-diabetic hormone exclusively produced by adipose tissue and released into the circulation that regulates glucose and lipid metabolism (reviewed in Pajvani and Scherer, Curr. Diab. Rep. 3: 207-213 (2003) herein incorporated by reference in its entirety). Specifically, adiponectin stimulates glucose utilization and fatty acid oxidation by activating the 5′-AMP-activated protein kinase (Yamauchi et al., Nat. Med. 8:1288-1295 (2002) herein incorporated by reference in its entirety). Adiponectin knock-out mice show delayed clearance of free fatty acids (FFA) in plasma, a high level of TNFα, and severe diet-induced insulin resistance (Maeda et al., Nat. Med. 8:731-737 (2002) herein incorporated by reference in its entirety).

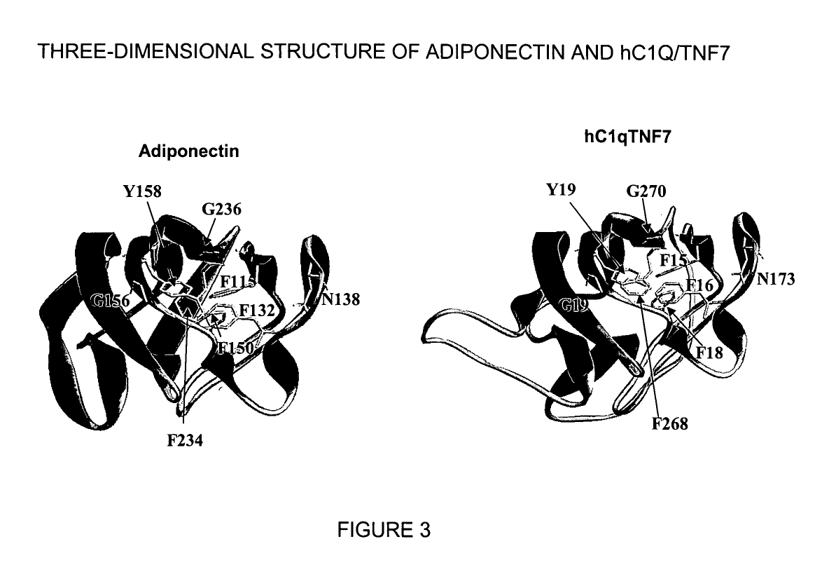

Structurally, adiponectin contains a leading signal peptide, a collagen-like region, and a C-terminal C1q domain. The 3D crystal structure of the C1q domain of adiponectin shows a significant similarity to that of TNFα indicating an evolutionary relationship between the C1q-related proteins and TNF family members (Shapiro and Scherer, 1998, supra). In the crystal structure of adiponectin, the protein adopts a prototypic 10 β-strand jelly roll with the eight invariant C1q domain residues found within the center of the structure (FIG. 3). Among these eight residues, all five aromatic residues are packed in the central hydrophobic core. The location of these eight residues is similar in hC1Q/TNF7 (FIG. 3). These highly conserved residues may play important roles in the formation or stabilization of the hydrophobic core of the C1q domain structure. However, in the TNF family, which shares a highly similar folding topology, three out of five of the aromatic residues are not conserved (Shapiro and Scherer, 1998, supra). Thus, it is possible that these invariant C1q residues also play roles in maintaining a distinctive architecture or surface necessary in the function of all C1q proteins that clearly differs from the requirements of the related TNF family of proteins.

The prototypic C1qTNF protein was identified by homology-based searches for TNF paralogs. Murine C1qTNF3 was identified by suppression substrative hybridization between TGFβ-1-treated and untreated cells, and was also named CORS26 for “collagenous repeat-containing sequence of 26 kD” (Maeda et al., J. Biol. Chem. 276:3628-3634 (2001) herein incorporated by reference in its entirety). It is expressed primarily in the cartilage primordium or developing cartilage in embryonal mouse tissues in vivo. The human ortholog of C1TNF3 mRNA is expressed in white adipose tissue as well as in osteosarcoma and chondroblastoma cells (Schaffler et al., Biochim. Biophys. Acta 1628:64-70 (2003) herein incorporated by reference in its entirety). Thus, C1qTNF3 may be a signaling molecule produced by pre-chondrocytic mesenchymal cells or early chondrocytes during skeletal development. Furthermore, overexpression of murine C1qTNF3 in C3H10T1/2 cells enhanced the cell growth/proliferation indicating that C1qTNF3 acts as a growth factor. Because of the homology between the C1q and TNF families, and since TNFs have monospecific receptors (TNFRs), C1qTNF3 and by analogy, hC1Q/TNF7, may signal through a TNFR or TNFR-like receptor.

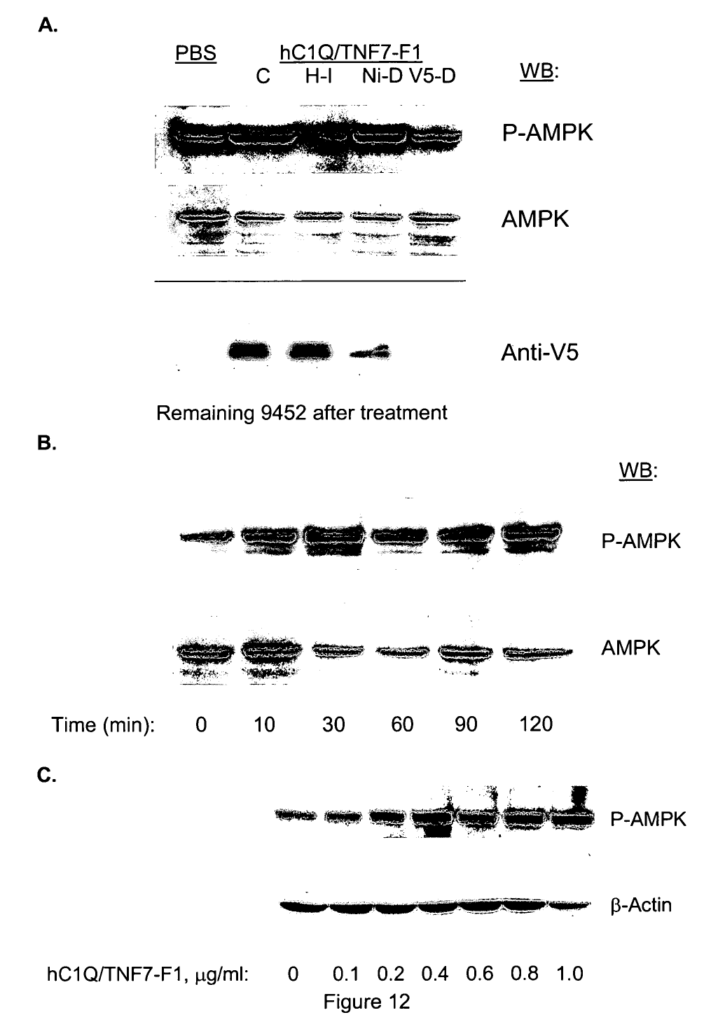

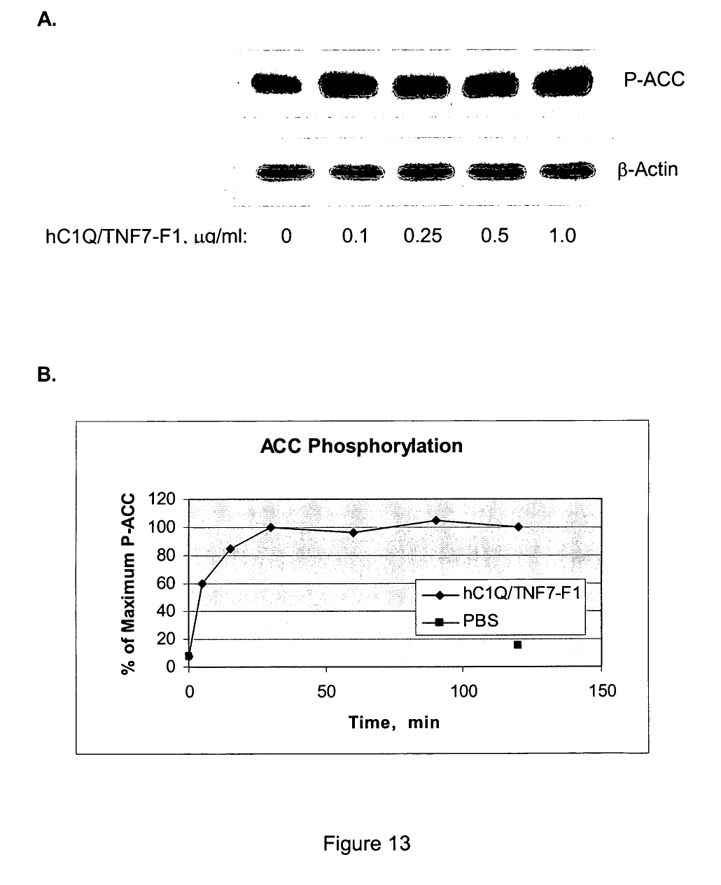

Sequence comparison indicates high homology between hC1Q/TNF7 and adiponectin (43% amino acid sequence homology) and with murine C1Q/TNF2 (72%). Previous studies revealed that adiponectin stimulates glucose utilization and fatty acid oxidation in C2C12 myoblasts as well as in isolated extensor digitorum longus muscle by activating AMPK as well as ACC signaling pathways (Tomas et al., Proc. Natl. Acad. Sci. USA 99:16309-16313 (2002) herein incorporated by reference in its entirety). ACC is a downstream effector of the AMPK signaling pathway. AMPK phosphorylation induces phosphorylation of ACC which in turn inhibits ACC activities and subsequently induces decreased cellular fatty acid synthesis and increased β-oxidation of fatty acids. Furthermore, recent studies suggest that mC1Q/TNF2 possesses similar in vitro biological activities as those of adiponectin with regard to stimulating glycogen accumulation and fatty acid oxidation (Wong et al., Proc. Natl. Acad. Sci. USA 101:10302-10307 (2004) herein incorporated by reference in its entirety). hC1Q/TNF7 also activates AMPK and ACC signaling pathways (see FIGS. 12-13 and Example 9); therefore hC1Q/TNF7 may share similar functions as adiponectin and mC1Q/TNF2.

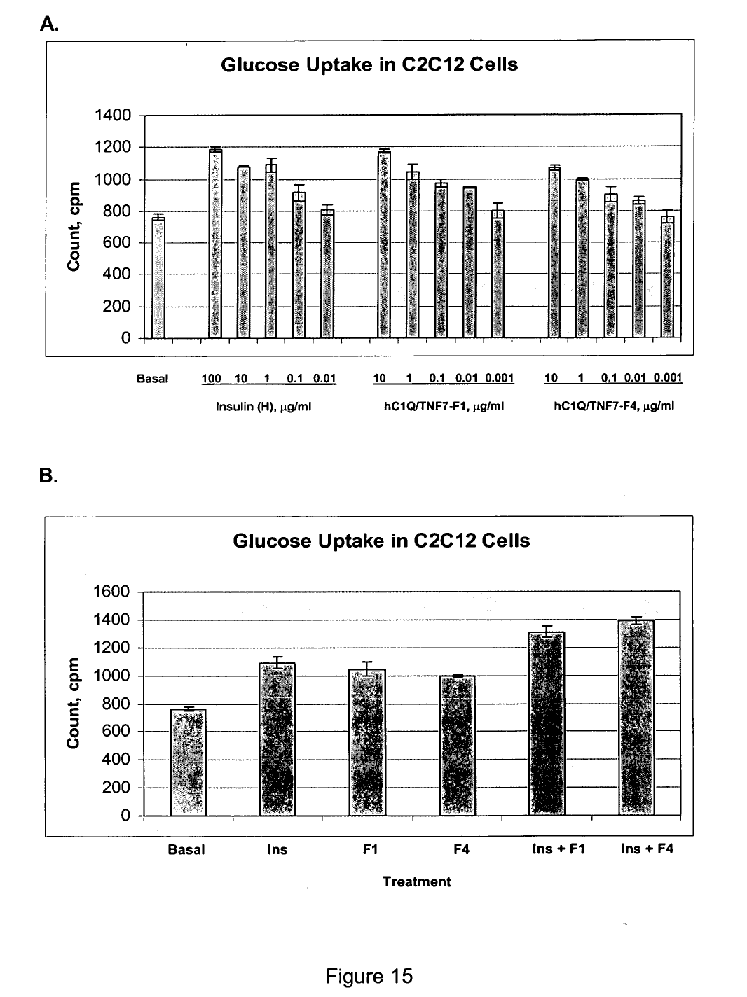

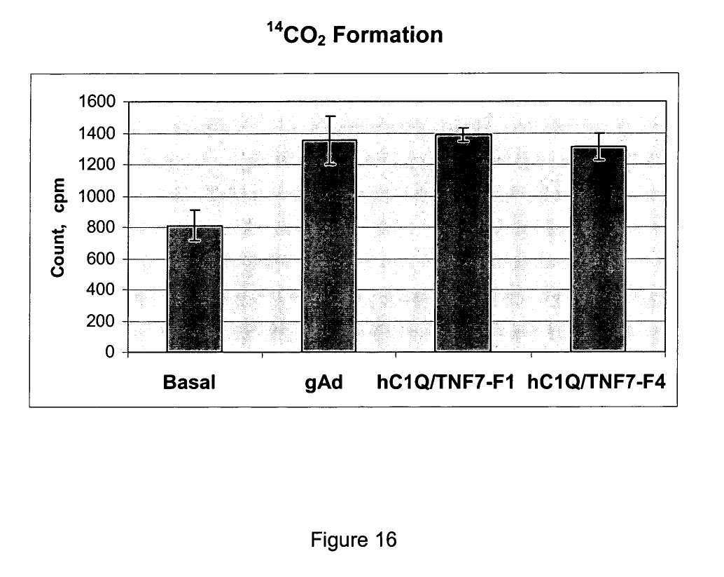

Increased AMPK phosphorylation is an indication of activation of AMPK signaling pathway, which results in increased glucose uptake and glycogen accumulation in muscle and myoblasts (Combs et al., J. Clin. Invest. 108:1875-1881 (2001) herein incorporated by reference in its entirety). In contrast, increased ACC phosphorylation inactivates ACC activity, resulting in increased β-fatty acid oxidation and decreased fatty acid synthesis (Freubis et al., Proc. Natl. Acad. Sci. USA 98:2005-2010 (2001) herein incorporated by reference in its entirety). As seen in FIGS. 15 and 16, hC1Q/TNF7 increases glucose uptake and fatty acid oxidation due to AMPK activation and increased ACC phosphorylation.

hC1Q/TNF7 polypeptides and polynucleotides of the invention may be used to stimulate growth of fatty and/or lean body mass. They may also be used in the treatment of wasting disorders, including cachexia resulting from a variety of chronic illnesses including cancer, AIDS, chronic obstructive pulmonary disease (COPD), advanced kidney disease, advanced cardiovascular disease, Crohn's disease, tuberculosis, severe infections, viral or bacterial diseases, autoimmune diseases, including rheumatoid arthritis and systemic lupus erythematosus (SLE), treatment-induced cachexia, for example from chemotherapeutic treatment, long-term convalescence, coma, and eating disorders, including anorexia and bulimia. In addition, hC1Q/TNF7 polypeptides and polynucleotides can be used to treat diseases and disorders that have impaired growth, such as genetic diseases of dwarfism, autoimmune diseases, including juvenile idiopathic arthritis, inflammatory bowel diseases, including growth failure associated with pediatric Crohn's disease, and other growth hormone deficiencies, including the natural decline in growth hormone associated with ageing.

hC1Q/TNF7 polypeptides and polynucleotides can be used to treat wasting disorders, eating disorders, and growth deficiencies or used to stimulate growth in non-human mammals, including pets, farm animals, and veterinary animals.

4.1 Definitions

In describing the present invention the following terms will be employed and are intended to be defined as indicated below.

It must be noted that as used herein and in the appended claims, the singular forms “a”, “an” and “the” include plural references unless the context clearly dictates otherwise.

The term “cachexia” refers to a weight loss condition occurring in patients with advanced illness. Cachexia is associated with a variety of chronic illnesses including cancer, AIDS, chronic obstructive pulmonary disease (COPD), advanced kidney disease, advanced cardiovascular disease, Crohn's disease, tuberculosis, severe infections, viral or bacterial diseases, and autoimmune diseases, including rheumatoid arthritis and systemic lupus erythematosus (SLE). Cachexia is a condition generally typified by abnormally high weight loss, including fatty tissue (“fatty body mass”), muscle tissue and even bone (collectively referred to as “lean body mass”). Additional symptoms of cachexia include anorexia (loss of appetite), asthenia (weakness), and anemia (drop in hemoglobin level).

The term “fragment” refers to a polypeptide derived from the native hC1Q/TNF7 that does not include the entire sequence of hC1Q/TNF7. Such a fragment may be a truncated version of the full-length molecule, as well as an internally deleted polypeptide. Preferably, a fragment comprises at least ten (10) consecutive amino acids of a polypeptide of the invention. A hC1Q/TNF7 fragment may have hC1Q/TNF7 bioactivity as determined by the effect of hC1Q/TNF7 on stimulating body mass growth, as described herein.

The term “analog” refers to derivatives of the reference molecule. The analog may retain biological activity, as described above. In general, the term “analog” refers to compounds having a native polypeptide sequence and structure with one or more amino acid additions, substitutions (generally conservative in nature) and/or deletions, relative to the native molecule, so long as the modifications do not destroy activity. Preferably, the analog has at least the same biological activity as the parent molecule, and may even display enhanced activity over the parent molecule. Methods for making polypeptide analogs are known in the art. Particularly preferred analogs include substitutions that are conservative in nature, i.e., those substitutions that take place within a family of amino acids that are related in their side chains. Specifically, amino acids are generally divided into four families: (1) acidic: aspartate and glutamate; (2) basic: lysine, arginine, histidine; (3) non-polar: alanine, valine, leucine, isoleucine, proline, phenylalanine, methionine, tryptophan; and (4) uncharged polar: glycine, asparagine, glutamine, cysteine, serine, threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid will preserve the biological activity of hC1Q/TNF7.

Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant analogs encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

The term “derivative” refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins.

The terms “polypeptide” and “protein” refer to a polymer of amino acid residues and are not limited to a minimum length of the product. The terms also include, unless otherwise indicated, modifications of the polypeptide that do not change the sequence of amino acids, for example, glycosylated, acetylated and phosphorylated forms. A polypeptide or protein, for purposes of the present invention, may be synthetically or recombinantly produced, as well as isolated from natural sources.

By “purified” and “isolated” is meant, when referring to a polypeptide or polynucleotide, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present in the sample. In one embodiment, the polynucleotide or polypeptide is purified such that it constitutes at least 95% by weight of the indicated biological macromolecules present but water, buffers, and other small molecules, especially molecules having a molecular weight of less than 1000 daltons, can be present.

An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “translated protein coding portion” means a sequence which encodes for the full length protein which may include any leader sequence or a processing sequence.

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

By a “recombinant polypeptide” is intended a polypeptide which has been prepared by recombinant DNA techniques as described herein. In general, the gene coding for the desired polypeptide is cloned and then expressed in transformed organisms, as described farther below. The host organism expresses the foreign gene to produce the polypeptide under expression conditions. Alternatively, the promoter controlling expression of an endogenous polypeptide can be altered to render a recombinant polypeptide.

The term “active” refers to those forms of the polypeptide that retain the biologic and/or immunologic activities of any naturally occurring polypeptide. According to the invention, the terms “biologically active” or “biological activity” refer to a protein or peptide having structural, regulatory or biochemical functions of a naturally occurring molecule. Likewise “biologically active” or “biological activity” refers to the capability of the natural, recombinant or synthetic hC1Q/TNF7 peptide, or any peptide thereof, to induce a specific biological response in appropriate animals or cells and to bind with specific antibodies.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum. “Secreted” proteins are also intended to include proteins containing non-typical signal sequences (e.g. Interleukin-1 Beta, see Krasney and Young (1992) Cytokine 4(2):134-143) and factors released from damaged cells (e.g. Interleukin-1 Receptor Antagonist, see Arend et. al. (1998) Annu. Rev. Immunol. 16:27-55)

The term “polynucleotide” or “nucleic acid molecule” as used herein refers to a polymeric form of nucleotides of any length, either ribonucleotides or deoxyribonucleotides. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment”, “portion,” or “segment” or “probe” or “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1-3, 5, 7, 9, 11-14, 38-53, 55, 57, 59, 61, 63, 65-66, 68, 70, 72, or 74.

Probes may, for example, be used to determine whether specific mRNA molecules are present in a cell or tissue or to isolate similar nucleic acid sequences from chromosomal DNA as described by Walsh et al. (Walsh, P. S. et al., 1992, PCR Methods Appl 1:241-250). They may be labeled by nick translation, Klenow fill-in reaction, PCR, or other methods well known in the art. Probes of the present invention, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

The nucleic acid sequences of the present invention also include the sequence information from any of the nucleic acid sequences of SEQ ID NO: 1-3, 5, 7, 9, 11-14, 38-53, 55, 57, 59, 61, 63, 65-66, 68, 70, 72, or 74. The sequence information can be a segment of SEQ ID NO: 1-3, 5, 7, 9, 11-14, 38-53, 55, 57, 59, 61, 63, 65-66, 68, 70, 72, or 74 that uniquely identifies or represents the sequence information of SEQ ID NO: 1-3, 5, 7, 9, 11-14, 38-53, 55, 57, 59, 61, 63, 65-66, 68, 70, 72, or 74. One such segment can be a twenty-mer nucleic acid sequence because the probability that a twenty-mer is fully matched in the human genome is 1 in 300. In the human genome, there are three billion base pairs in one set of chromosomes. Because 4²⁰ possible twenty-mers exist, there are 300 times more twenty-mers than there are base pairs in a set of human chromosomes. Using the same analysis, the probability for a seventeen-mer to be fully matched in the human genome is approximately 1 in 5. When these segments are used in arrays for expression studies, fifteen-mer segments can be used. The probability that the fifteen-mer is fully matched in the expressed sequences is also approximately one in five because expressed sequences comprise less than approximately 5% of the entire genome sequence.

Similarly, when using sequence information for detecting a single mismatch, a segment can be a twenty-five mer. The probability that the twenty-five mer would appear in a human genome with a single mismatch is calculated by multiplying the probability for a full match (1÷4²⁵) times the increased probability for mismatch at each nucleotide position (3×25). The probability that an eighteen mer with a single mismatch can be detected in an array for expression studies is approximately one in five. The probability that a twenty-mer with a single mismatch can be detected in a human genome is approximately one in five.

The term “open reading frame,” ORF, means a series of nucleotide triplets coding for amino acids without any termination codons and is a sequence translatable into protein.

The terms “operably linked” or “operably associated” refer to functionally related nucleic acid sequences. For example, a promoter is operably associated or operably linked with a coding sequence if the promoter controls the transcription of the coding sequence. While operably linked nucleic acid sequences can be contiguous and in the same reading frame, certain genetic elements e.g. repressor genes are not contiguously linked to the coding sequence but still control transcription/translation of the coding sequence.

The terms “recombinant DNA molecule,” or “recombinant polynucleotide” are used herein to refer to a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature. Thus, the term encompasses “synthetically derived” nucleic acid molecules.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind or it may be “complete” such that total complementarity exists between the single stranded molecules. The degree of complementarity between the nucleic acid strands has significant effects on the efficiency and strength of the hybridization between the nucleic acid strands.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.).

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 90% sequence identity. Substantially equivalent nucleotide sequence of the invention can have lower percent sequence identities, taking into account, for example, the redundancy or degeneracy of the genetic code. Preferably, nucleotide sequence has at least about 65% identity, more preferably at least about 75% identity, and most preferably at least about 95% identity. For the purposes of the present invention, sequences having substantially equivalent biological activity and substantially equivalent expression characteristics are considered substantially equivalent. For the purposes of determining equivalence, truncation of the mature sequence (e.g., via a mutation which creates a spurious stop codon) should be disregarded. Sequence identity may be determined, e.g., using the Jotun Hein method (Hein, J. (1990) Methods Enzymol. 183:626-645). Identity between sequences can also be determined by other methods known in the art, e.g. by varying hybridization conditions.

The term “vector” refers to a nucleic acid molecule capable of transporting another nucleic acid to which it has been linked. The term “expression vector” includes plasmids, cosmids or phages capable of synthesizing the hC1Q/TNF7 protein encoded by the respective recombinant gene carried by the vector. Preferred vectors are those capable of autonomous replication and expression of nucleic acids to which they are linked.

The term “transformation” means introducing DNA into a suitable host cell so that the DNA is replicable, either as an extrachromosomal element, or by chromosomal integration.

The term “transfection” refers to the taking up of an expression vector by a suitable host cell, whether or not any coding sequences are in fact expressed. The term “infection” refers to the introduction of nucleic acids into a suitable host cell by use of a virus or viral vector.

The term “transcriptional regulatory elements” and transcriptional regulatory sequences” are used interchangeably to refer to DNA sequences necessary for the expression of an operably linked coding sequence in a particular organism. The control sequences that are suitable for prokaryotes, for example, include a promoter, optionally an operator sequence, and a ribosome binding site. Eukaryotic cells are known to utilize promoters, enhancers, splicing signals and polyadenylation signals. These terms are intended to encompass all elements that promote or regulate transcription, including promoters, core elements required for basic interaction of RNA polymerase and transcription factors, upstream elements, enhancers, and response elements (Lewin, “Genes V” (Oxford University Press, Oxford) pages 847-873).

A coding sequence is “under the control” of transcriptional and translational control sequences in a cell when RNA polymerase transcribes the coding sequence into mRNA, which is then optionally trans-RNA spliced and translated into the protein encoded by the coding sequence.

The term “tissue-specific promoter” means a nucleotide sequence that serves as a promoter, i.e. regulates expression of a selected DNA sequence operably linked to the promoter, and which effects the expression of the selected DNA sequence in specific cells.

The term “expression modulating fragment,” EMF, means a series of nucleotides that modulates the expression of an operably linked ORF or another EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs is nucleic acid fragments which induce the expression of an operably linked ORF in response to a specific regulatory factor or physiological event.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “totipotent” refers to the capability of a cell to differentiate into all of the cell types of an adult organism.

The term “pluripotent” refers to the capability of a cell to differentiate into a number of differentiated cell types that are present in an adult organism. A pluripotent cell is restricted in its differentiation capability in comparison to a totipotent cell.

The term “non-human mammal” refers to all members of the class Mammalia except humans. “Mammal” refers to any animal classified as a mammal, including humans, domestic and farm animals, and zoo, sports, or pet animals, such as a mouse, rat, rabbit, pig, sheep, goat, cattle and higher primates.

The terms “treat” or “treatment” refer to both therapeutic and prophylactic or preventative measures, wherein the object is to prevent or lessen an undesired physiological change or condition, such as cachexia. For the purposes of this invention, beneficial or desired clinical results include, but are not limited to alleviation of symptoms, diminishment of extent of the disease, stabilized state of the disease, whether detectable or undetectable.

A “disorder” is any condition that would benefit from treatment with a molecule identified using the cell-based method of the invention. This includes chronic and acute disorders or diseases including those pathological conditions which predispose the mammal to the disorder in question. Non-limiting examples of disorders to be treated herein include cachexia, wasting disorders, eating disorders, and growth disorders. A preferred disorder to be treated in accordance with the present invention is cachexia.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an effective amount of a hC1Q/TNF7 fragment for use with the present methods is an amount sufficient to stimulate body mass growth. Such amounts are described below. An appropriate “effective” amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.0 to 8.0 inclusive. Preferred physiological pH is in the range of approximately 7.2 to 7.6 inclusive.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

4.2 Compositions of the Invention

4.2.1 Nucleic Acid Compositions

The invention is based on the discovery that compositions comprising the polypeptide, hC1Q/TNF7, and the polynucleotides encoding the hC1Q/TNF7 polypeptide. Therefore, the use of these compositions for the diagnosis and treatment of conditions wherein the reversal of loss of body mass, especially lean body mass, is contemplated.

The isolated polynucleotides of the invention include, but are not limited to a polynucleotide comprising any of the nucleotide sequences of SEQ ID NO: 1-3, 5, 7, 9, 11-14, 38-53, 55, 57, 59, 61, 63, 65-66, 68, 70, 72, or 74; a fragment of SEQ ID NO: 1-3, 5, 7, 9, 11-14, 38-53, 55, 57, 59, 61, 63, 65-66, 68, 70, 72, or 74; a polynucleotide comprising the full length protein coding sequence of SEQ ID NO: 3, 5, 9, 53, 55, 65, or 66; (for example coding for SEQ ID NO: 4, 8, 10, 54, 56, or 67); and a polynucleotide comprising the nucleotide sequence encoding the mature protein coding sequence of the polypeptide of SEQ ID NO: 4, 8, 10, 54, 56, or 67. The polynucleotides of the present invention also include, but are not limited to, a polynucleotide that hybridizes under stringent conditions to (a) the complement of any of the nucleotides sequences of SEQ ID NO: 1-3, 5, 7, 9, 11-14, 38-53, 55, 57, 59, 61, 63, 65-66, 68, 70, 72, or 74; (b) a polynucleotide encoding any one of the polypeptides of SEQ ID NO: 4, 6, 8, 10, 54, 56, 58, 60, 62, 64, 67, 69, 71, 73, or 75; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NO: 4, 6, 8, 10, 54, 56, 58, 60, 62, 64, 67, 69, 71, 73, or 75. Domains of interest include extracellular, transmembrane, or cytoplasmic domains, or combinations thereof; and catalytic and substrate binding domains.

The polynucleotides of the invention include naturally occurring or wholly or partially synthetic DNA, e.g., cDNA and genomic DNA, and RNA, e.g., mRNA. The polynucleotides may include the entire coding region of the cDNA or may represent a portion of the coding region of the cDNA.

The present invention also provides compositions comprising genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides of SEQ ID NO: 1-3, 5, 7, 9, 11-14, 38-53, 55, 57, 59, 61, 63, 65-66, 68, 70, 72, or 74 can be obtained by screening appropriate cDNA or genomic DNA libraries under suitable hybridization conditions using any of the polynucleotides of SEQ ID NO: 1-3, 5, 7, 9, 11-14, 38-53, 55, 57, 59, 61, 63, 65-66, 68, 70, 72, or 74 or a portion thereof as a probe. Alternatively, the polynucleotides of SEQ ID NO: 1-3, 5, 7, 9, 11-14, 38-53, 55, 57, 59, 61, 63, 65-66, 68, 70, 72, or 74 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to a polynucleotide recited above.

Included within the scope of the nucleic acid sequences of the invention are nucleic acid sequence fragments that hybridize under stringent conditions to any of the nucleotide sequences of SEQ ID NO: 1-3, 5, 7, 9, 11-14, 38-53, 55, 57, 59, 61, 63, 65-66, 68, 70, 72, or 74, or complements thereof, which fragment is greater than about 5 nucleotides, preferably 7 nucleotides, more preferably greater than 9 nucleotides and most preferably greater than 17 nucleotides. Fragments of, e.g. 15, 17, or 20 nucleotides or more that are selective for (i.e. specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof. Allelic and species variations can be routinely determined by comparing the sequence provided in SEQ ID NO: 1-3, 5, 7, 9, 11-14, 38-53, 55, 57, 59, 61, 63, 65-66, 68, 70, 72, or 74, a representative fragment thereof, or a nucleotide sequence at least 90% identical, preferably 95% identical, to SEQ ID NO: 1-3, 5, 7, 9, 11-14, 38-53, 55, 57, 59, 61, 63, 65-66, 68, 70, 72, or 74 with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids of the present invention can be obtained by searching a database using an algorithm or a program. Preferably, a BLAST which stands for Basic Local Alignment Search Tool is used to search for local sequence alignments (Altshul, S. F. J Mol. Evol. 36 290-300 (1993) and Altschul S. F. et al. J. Mol. Biol. 21:403-410 (1990))

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encodes proteins which are identical, homologous or related to that encoded by the polynucleotides.

The nucleic acid sequences of the invention are further directed to sequences which encode analogs of the described nucleic acids. These amino acid sequence analogs may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence analogs are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells and sequences such as poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells et al., Gene 34:315 (1985); and other mutagenesis techniques well known in the art, such as, for example, the techniques in Sambrook et al., supra, and Current Protocols in Molecular Biology, Ausubel et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, polynucleotide sequences comprising the mature protein coding sequences, coding for any one of SEQ ID NO: 4, 8, 10, 54, 56, or 67, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the nucleotide sequences of SEQ ID NO: 1-3, 5, 7, 9, 11-14, 38-53, 55, 57, 59, 61, 63, 65-66, 68, 70, 72, or 74 or a fragment thereof or any other hC1Q/TNF7 polynucleotides. In one embodiment, the recombinant constructs of the present invention comprise a vector, such as a plasmid or viral vector, into which a nucleic acid having any of the nucleotide sequences of SEQ ID NO: 1-3, 5, 7, 9, 11-14, 38-53, 55, 57, 59, 61, 63, 65-66, 68, 70, 72, or 74 or a fragment thereof is inserted. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia). In one embodiment, the nucleic acid of SEQ ID NO: 3 is inserted in the pIntron vector of the invention as described in the examples.

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

In addition to the use of expression vectors in the practice of the present invention, the present invention further includes novel expression vectors comprising promoter elements operatively linked to polynucleotide sequences encoding a protein of interest. One example of such a vector is the pcDNA/Intron vector, which is described in Example 5.

4.2.2 Hosts

The present invention further provides host cells genetically engineered with the vectors of this invention, which may be, for example, a cloning vector or an expression vector that contain the polynucleotides of the invention. For example, such host cells may contain nucleic acids of the invention introduced into the host cell using known transformation, transfection or infection methods. The vector may be, for example, in the form of a plasmid, a viral particle, a phage, etc. The engineered host cells can be cultured in conventional nutrient media modified as appropriate for activating promoters, selecting transformants or amplifying hC1Q/TNF7 genes. The culture conditions, such as temperature, pH, and the like, are those previously used with the host cell selected for expression, and will be apparent to the ordinarily skilled artisan. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF) or can be used to produce a heterologous protein under the control of the EMF.

Any host/vector system can be used to express one or more of the hC1Q/TNF7 polypeptides. These include, but are not limited to, eukaryotic hosts such as HeLa cells, Cv-1 cell, COS cells, human embryonic kidney (HEK) 293 cells, and Sf9 cells, as well as prokaryotic hosts such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa, HEK293 cells, and BHK cell lines. Mammalian expression vectors will comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, human epidermal A431 cells, human Colo205 cells, HEK293 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Preferably, hC1Q/TNF7 proteins are expressed in HEK293 cells, CHO cells or COS cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

4.2.3 Chimeric and Fusion Proteins

The invention also provides hC1Q/TNF7 chimeric or fusion proteins. As used herein, a hC1Q/TNF7 “chimeric protein” or “fusion protein” comprises a hC1Q/TNF7 polypeptide operatively-linked to a non-hC1Q/TNF7 polypeptide. A “hC1Q/TNF7 polypeptide” refers to a polypeptide having an amino acid sequence corresponding to a hC1Q/TNF7 protein, whereas a “non-hC1Q/TNF7 polypeptide” refers to a polypeptide having an amino acid sequence corresponding to a protein that is not substantially homologous to the hC1Q/TNF7 protein, e.g., a protein that is different from the hC1Q/TNF7 protein and that is derived from the same or a different organism. Within a hC1Q/TNF7 fusion protein the hC1Q/TNF7 polypeptide can correspond to all or a portion of a hC1Q/TNF7 protein. In one embodiment, a hC1Q/TNF7 fusion protein comprises at least one biologically active portion of a hC1Q/TNF7 protein. In another embodiment, a hC1Q/TNF7 fusion protein comprises at least two biologically active portions of a hC1Q/TNF7 protein. In yet another embodiment, a hC1Q/TNF7 fusion protein comprises at least three biologically active portions of a hC1Q/TNF7 protein. Within the fusion protein, the term “operatively-linked” is intended to indicate that the hC1Q/TNF7 polypeptide and the non-hC1Q/TNF7 polypeptide are fused in-frame with one another. The non-hC1Q/TNF7 polypeptide can be fused to the N-terminus or C-terminus of the hC1Q/TNF7 polypeptide.

In one embodiment, the fusion protein is a GST-hC1Q/TNF7 fusion protein in which the hC1Q/TNF7 sequences are fused to the C-terminus of the GST (glutathione S-transferase) sequences. Such fusion proteins can facilitate the purification of recombinant hC1Q/TNF7 polypeptides. In another embodiment, the fusion protein is a hC1Q/TNF7 protein containing a heterologous signal sequence at its N-terminus. In certain host cells (e.g., mammalian host cells), expression and/or secretion of hC1Q/TNF7 can be increased through use of a heterologous signal sequence. Preferably, the hC1Q/TNF7 polypeptide is fused with a V5-His tag for easy detection with an anti-V5 antibody and for rapid purification as described in the examples.

A hC1Q/TNF7 chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A hC1Q/TNF7-encoding nucleic acid can be cloned into such an expression vector such that the fusion moiety is linked in-frame to the hC1Q/TNF7 protein.

4.2.4 Polypeptide Compositions

The pharmaceutical compositions of the invention comprise isolated hC1Q/TNF7 polypeptides that include, but are not limited to, a polypeptide comprising: the amino acid sequence set forth as any one of SEQ ID NO: 4, 6, 8, 10, 54, 56, 58, 60, 62, 64, 67, 69, 71, 73, or 75, or an amino acid sequence encoded by any one of the nucleotide sequences SEQ ID NO: 1-3, 5, 7, 9, 11-14, 38-53, 55, 57, 59, 61, 63, 65-66, 68, 70, 72, or 74. Polypeptides of the invention also include polypeptides preferably with biological or immunological activity that are encoded by: (a) a polynucleotide having any one of the nucleotide sequences set forth in SEQ ID NO: 1-3, 5, 7, 9, 11-14, 38-53, 55, 57, 59, 61, 63, 65-66, 68, 70, 72, or 74 or (b) polynucleotides encoding any one of the amino acid sequences set forth as SEQ ID NO: 4, 6, 8, 10, 54, 56, 58, 60, 62, 64, 67, 69, 71, 73, or 75, or (c) polynucleotides that hybridize to the complement of the polynucleotides of either (a) or (b) under stringent hybridization conditions. The invention also provides biologically active or immunologically active variants of any of the amino acid sequences set forth as SEQ ID NO: 4, 6, 8, 10, 54, 56, 58, 60, 62, 64, 67, 69, 71, 73, or 75; and “substantial equivalents” thereof (e.g., with at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99%, most typically at least 99% amino acid identity) that retain biological activity. Polypeptides encoded by allelic variants may have a similar, increased, or decreased activity compared to polypeptides comprising SEQ ID NO: 4, 6, 8, 10, 54, 56, 58, 60, 62, 64, 67, 69, 71, 73, or 75.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are incorporated herein by reference. Such fragments may be fused to carrier molecules such as immunoglobulins for many purposes, including increasing the valency of protein binding sites.

The present invention also provides both full-length and mature forms (for example, without a signal sequence or precursor sequence) of the disclosed proteins. The nucleotide sequence encoding polypeptides of the invention is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells which are transformed with hC1Q/TNF7-encoding DNA to produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

The protein of the invention may also be expressed as a product of transgenic animals, e.g., as a component of the milk of transgenic cows, goats, pigs, or sheep which are characterized by somatic or germ cells containing a nucleotide sequence encoding the protein.

The proteins provided herein also include proteins characterized by amino acid sequences similar to those of purified proteins but into which modification are naturally provided or deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method which involved systematic substitution of single or strings of amino acids with alanine, followed by testing the resulting alanine-containing variant for biological activity. This type of analysis determines the importance of the substituted amino acid(s) in biological activity. Regions of the protein that are important for protein function may be determined by the eMATRIX program.

Other fragments and derivatives of the sequences of proteins which would be expected to retain protein activity in whole or in part and are useful for screening or other immunological methodologies may also be easily made by those skilled in the art given the disclosures herein. Such modifications are encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His (V5-His) tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The polypeptides of the invention include hC1Q/TNF7 analogs. This embraces fragments of hC1Q/TNF7 polypeptide, as well as hC1Q/TNF7 polypeptides which comprise one or more amino acids deleted, inserted, or substituted. Also, analogs of the hC1Q/TNF7 polypeptide of the invention embrace fusions of the hC1Q/TNF7 polypeptides or modifications of the hC1Q/TNF7 polypeptides, wherein the hC1Q/TNF7 polypeptide or analog is fused to another moiety or moieties, e.g., targeting moiety or another therapeutic agent. Such analogs may exhibit improved properties such as activity and/or stability. Examples of moieties which may be fused to the hC1Q/TNF7 polypeptide or an analog include, for example, targeting moieties which provide for the delivery of polypeptide to target cells. Other moieties which may be fused to hC1Q/TNF7 polypeptide include therapeutic agents which are used for treatment, for example cytokines or other medications, of wasting disorders, and other conditions as recited herein.

4.2.5 Gene Therapy

The invention provides gene therapy to treat the diseases cited herein. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84 (1990); and Miller, Nature, 357: 455-460 (1992).

As discussed above, a “vector” is any means for the transfer of a nucleic acid according to the invention into a host cell. Preferred vectors are viral vectors, such as retroviruses, herpes viruses, adenoviruses and adeno-associated viruses. Thus, a gene or nucleic acid sequence encoding a hC1Q/TNF7 protein or polypeptide domain fragment thereof is introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both.

Viral vectors commonly used for in vivo or ex vivo targeting and therapy procedures are DNA-based vectors and retroviral vectors. Methods for constructing and using viral vectors are known in the art [see, e.g., Miller and Rosman, BioTechniques 7:980-990 (1992)]. Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsulating the viral particles.

DNA viral vectors include an attenuated or defective DNA virus, such as but not limited to herpes simplex virus (HSV), papillomavirus, Epstein-Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector [Kaplitt et al., Molec. Cell. Neurosci. 2:320-330 (1991)], defective herpes virus vector lacking a glyco-protein L gene [Patent Publication RD 371005 A], or other defective herpes virus vectors [International Patent Publication No. WO 94/21807, published Sep. 29, 1994; International Patent Publication No. WO 92/05263, published Apr. 2, 1994]; an attenuated adenovirus vector, such as the vector described by Strafford-Perricaudet et al. [J. Clin. Invest. 90:626-630 (1992); see also La Salle et al., Science 259:988-990 (1993)]; and a defective adeno-associated virus vector [Samulski et al., J. Virol. 61:3096-3101 (1987); Samulski et al., J. Virol. 63:3822-3828 (1989); Lebkowski et al., Mol. Cell. Biol. 8:3988-3996 (1988)].

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors [see, e.g., Wilson, Nature Medicine (1995)]. In addition, it is advantageous to employ a viral vector that is engineered to express a minimal number of antigens.

Promoters that may be used in the present invention include both constitutive promoters and regulated (inducible) promoters. The promoter may be naturally responsible for the expression of the nucleic acid. It may also be from a heterologous source. In particular, it may be promoter sequences of eukaryotic or viral genes. For example, it may be promoter sequences derived from the genome of the cell which it is desired to infect. Likewise, it may be promoter sequences derived from the genome of a virus, including the adenovirus used. In this regard, there may be mentioned, for example, the promoters of the E1A, MLP, CMV and RSV genes and the like.

In addition, the promoter may be modified by addition of activating or regulatory sequences or sequences allowing a tissue-specific or predominant expression (enolase and GFAP promoters and the like). Moreover, when the nucleic acid does not contain promoter sequences, it may be inserted, such as into the virus genome downstream of such a sequence.

Some promoters useful for practice of this invention are ubiquitous promoters (e.g., HPRT, vimentin, actin, tubulin), intermediate filament promoters (e.g., desmin, neurofilaments, keratin, GFAP), therapeutic gene promoters (e.g., MDR type, CFTR, factor VIII), tissue-specific promoters (e.g., actin promoter in smooth muscle cells), promoters which are preferentially activated in dividing cells, promoters which respond to a stimulus (e.g., steroid hormone receptor, retinoic acid receptor), tetracycline-regulated transcriptional modulators, cytomegalovirus immediate-early, retroviral LTR, metallothionein, SV-40, E1a, and MLP promoters. Tetracycline-regulated transcriptional modulators and CMV promoters are described in WO 96/01313, U.S. Pat. Nos. 5,168,062 and 5,385,839, the contents of which are incorporated herein by reference.

Thus, the promoters which may be used to control gene expression include, but are not limited to, the cytomegalovirus (CMV) promoter, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlaufet al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. In addition to the use of viral vectors in the practice of the present invention, the present invention further includes a novel vector comprising operator and promoter elements operatively linked to polynucleotide sequences encoding a protein of interest.

4.2.6 Cell-Based Method for in vivo Biological Activity

The present invention provides a cell-based method that allows for identification of in vivo biological phenotypes of target genes. The cell-based method of the invention provides rapid screening of numerous gene products (i.e. target gene products) for biological activity. The principle behind the technique is straightforward and results in the circulation of the target protein in the blood of the host. The cell-based method of the invention has several advantageous hallmarks including: 1) rapid generation of cell lines expressing and secreting high levels of target protein; 2) fast turn-around on the determination of biological activity in vivo, typically 2-3 weeks; 3) multi-parametric analysis on serum parameters; 4) rapid inspection of pathology on multiple organs; and 5) high-throughput analysis of secreted proteins. The method is described in detail with respect to hC1Q/TNF7 in Examples 5 and 6. It is intended that this method may be applicable to any secreted protein.

A. Generation of Stable Pools or Clones:

In one embodiment, the target gene is cloned into the pIntron expression vector for high-level expression in eukaryotic cells. The eukaryotic expression vector pIntron is a modified expression vector obtained by introduction of an engineered chimeric intron derived from the pCI mammalian expression vector (Promega, Madison, Wis.) into the pcDNA3.1TOPO vector (Invitrogen Inc., Carlsbad, Calif.). A DNA fragment including the cytomegalovirus (CMV) immediate-early enhancer/promoter and a chimeric intron from the pCI vector is ligated into pcDNA3.1 (digested with BglII and KpnI) to create pIntron.

Generation of stable cell lines expressing the target genes of interest by transfecting 2-4 μg of plasmid DNA into HEK293 cells using the transfection reagent Fugene (Roche, Palo Alto, Calif.) according to manufacturer's instructions. The cells transfected with the plasmids of interest are allowed to express the protein that confers resistance to Geneticin (Invitrogen) for 24-48 hours prior to placing the cells under selection. Selection is performed by culturing the transfected cells in 1.5-2 mg of geneticin (G418) for 3-4 months. After a selection period of 2-3 weeks, the cells are tested for the production of the target protein by western blot analysis. Both untagged (wild-type) and tagged (i.e. V5-His, GST, etc.) target proteins can be analyzed according to this method. In a preferred embodiment, the target proteins are V5-His tagged and can be detected using an anti-V5 antibody. The level of protein expression and the appropriate size of the target molecule are determined by the intensity of the signal and its position on the western membrane in relation to molecular weight markers.

B. In Vivo Protein Expression:

In one embodiment, positive pools of cells expressing the target molecule are used for in vivo analysis after a 2-3 weeks of antibiotic selection. To facilitate expression in vivo in mice, the cell line HEK293 can be used for transfection for its ability to form tumors in immuno-compromised animals (e.g. Nude (Nu/Nu) mice). In alternate embodiments CHO cells or COS cells can be used to form solid tumors in Nude mice. Stable bulk pools for cells are expanded an harvested to provide enough cells for the administration of 20-30 million cells per mouse. Target gene expressing bulk pools of cells are administered subcutaneously to Nu/Nu mice (Charles River, Mass.) on the left hind flank. A suitable control us injection of a green fluorescent protein (GFP)-expressing HEK293 stable bulk pool. Tumor development occurs in injected mice for 3-4 weeks. Circulating levels of target protein are assessed by collecting blood from the mice by retro-orbital bleeding. The blood is then processed to obtain the serum component and analyzed by western to determine the level of target protein.

C. Phenotypic Analysis

Mice are euthanized and processed for analysis by removing as much blood as possible and harvesting organs, including lungs, liver, heart, kidney, spleen, colon, small intestine, skin, and tumor and processed for immunohistochemical analysis. Serum chemistry analytes are assayed, including albumin, alkaline phosphatase, amylase, bilirubin D, bilirubin Id, bilirubin T, BUN (blood urea nitrogen), cholesterol, creatine, GGT (gamma glutamyltransferase), glucose, LDH (lactate dehydrogenase), protein T, ALT (alanine transaminase), AST (aspartate aminotransferase), triglycerides, and CBC (complete blood count).

4.2.7 Cytokine and Cell Proliferation/Differentiation Activity

A polypeptide of the present invention may exhibit activity relating to cytokine, cell proliferation (either inducing or inhibiting) or cell differentiation (either inducing or inhibiting) activity or may induce production of other cytokines in certain cell populations. In addition, a polypeptide of the present invention may inhibit the activity of certain cytokines in specific cell populations. A polynucleotide of the invention can encode a polypeptide exhibiting such attributes. Many protein factors discovered to date, including all known cytokines, have exhibited activity in one or more factor-dependent cell proliferation assays, and hence the assays serve as a convenient confirmation of cytokine activity. The activity of therapeutic compositions of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e, CMK, HUVEC, and Caco. Therapeutic compositions of the invention can be used in the following:

Assays for T-cell or thymocyte proliferation include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., I. Immunol. 149:3778-3783, 1992; Bowman et al., I. Immunol. 152:1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human interleukin-γ, Schreiber, R. D. In Current Protocols in Immunology. J. E. e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J. E. Coligan eds. Vol 1 pp. 6.6.1-6.6.5, John Wiley and Sons, Toronto. 1991; Smith et al., Proc. Natl. Aced. Sci. U.S.A. 83:1857-1861, 1986; Measurement of human Interleukin 11—Bennett, F., Giannotti, J., Clark, S. C. and Turner, K. J. In Current Protocols in Immunology. J. E. Coligan eds. Vol 1 pp. 6.15.1 John Wiley and Sons, Toronto. 1991; Measurement of mouse and human Interleukin 9-Ciarletta, A., Giannotti, J., Clark, S. C. and Turner, K. J. In Current Protocols in Immunology. J. E. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405411, 1981; Takai et al., J. Immunol. 137:3494-3500, 1986; Takai et al., J. Immunol. 140:508-512, 1988.

4.2.8 Tissue Growth Activity

A polypeptide of the present invention also may be involved in bone, cartilage, tendon, ligament and/or nerve tissue growth or regeneration.

A polypeptide of the present invention which induces cartilage and/or bone growth in circumstances where bone is not normally formed has application in the healing of bone fractures and cartilage damage or defects in humans and other animals. In addition, a polypeptide of the present invention with bone and/or cartilage growth activity can be used to increase the stature of a patient with impaired growth or to replace or reverse the loss of bone density. Compositions of a polypeptide, antibody, binding partner, or other modulator of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A polypeptide of this invention may also be involved in attracting bone-forming cells, stimulating growth of bone-forming cells, or inducing differentiation of progenitors of bone-forming cells. Treatment of osteoporosis, osteoarthritis, bone degenerative disorders, or periodontal disease, such as through stimulation of bone and/or cartilage repair or by blocking inflammation or processes of tissue destruction (collagenase activity, osteoclast activity, etc.) mediated by inflammatory processes may also be possible using the composition of the invention.

Another category of tissue regeneration activity that may involve the polypeptide of the present invention is tendon/ligament formation. Induction of tendon/ligament-like tissue or other tissue formation in circumstances where such tissue is not normally formed has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

A composition of the present invention may also be useful for promoting or inhibiting differentiation of tissues described above from precursor tissues or cells; or for inhibiting the growth of tissues described above.

Assays for tissue generation activity include, without limitation, those described in: International Patent Publication No. WO95/16035 (bone, cartilage, tendon); International Patent Publication No. WO91/07491 (skin, endothelium).

4.2.9 Receptor Ligand Activity

A polypeptide of the present invention may also demonstrate activity as a soluble receptor, receptor ligand or inhibitor or agonist of receptor/ligand interactions. A polynucleotide of the invention can encode a polypeptide exhibiting such characteristics. Examples of such receptors and ligands include, without limitation, tumor necrosis factor receptors and their ligands, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses. Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a polypeptide of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley—Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

By way of example, the polypeptides of the invention may be used as a receptor for a ligand(s) thereby transmitting the biological activity of that ligand(s). Ligands may be identified through binding assays, affinity chromatography, dihybrid screening assays, BIAcore assays, gel overlay assays, or other methods known in the art.

Studies characterizing drugs or proteins as agonist or antagonist or partial agonists or a partial antagonist require the use of other proteins as competing ligands. The polypeptides of the present invention or ligand(s) thereof may be labeled by being coupled to radioisotopes, colorimetric molecules or a toxin molecules by conventional methods. (“Guide to Protein Purification” Murray P. Deutscher (ed) Methods in Enzymology Vol.182 (1990) Academic Press, Inc. San Diego). Examples of radioisotopes include, but are not limited to, tritium and carbon-14. Examples of colorimetric molecules include, but are not limited to, fluorescent molecules such as fluorescamine, or rhodamine or other colorimetric molecules. Examples of toxins include, but are not limited, to ricin.

4.2.10 Assay for Receptor Activity

The invention also provides methods to detect specific binding of a polypeptide e.g. a ligand or a receptor. The art provides numerous assays particularly useful for identifying previously unknown binding partners for receptor polypeptides of the invention. For example, expression cloning using mammalian or bacterial cells or dihybrid screening assays can be used to identify polynucleotides encoding binding partners. As another example, affinity chromatography with the appropriate immobilized polypeptide of the invention can be used to isolate polypeptides that recognize and bind polypeptides of the invention. There are a number of different libraries used for the identification of compounds, and in particular small molecules, that modulate (i.e., increase or decrease) biological activity of a polypeptide of the invention. Ligands for receptor polypeptides of the invention can also be identified by adding exogenous ligands, or cocktails of ligands to two cells populations that are genetically identical except for the expression of the receptor of the invention: one cell population expresses the receptor of the invention whereas the other does not. The response of the two cell populations to the addition of ligands(s) is then compared. Alternatively, an expression library can be co-expressed with a polypeptide of the invention in cells and assayed for an autocrine response to identify potential ligand(s). As still another example, BIAcore assays, gel overlay assays, or other methods known in the art can be used to identify binding partner polypeptides, including: (1) organic and inorganic chemical libraries, (2) natural product libraries, and (3) combinatorial libraries comprised of random peptides, oligonucleotides or organic molecules.

The role of downstream intracellular signaling molecules in the signaling cascade of the polypeptide of the invention can be determined. For example, a chimeric protein in which the cytoplasmic domain of the polypeptide of the invention is fused to the extracellular portion of a protein, whose ligand has been identified, is produced in a host cell. The cell is then incubated with the ligand specific for the extracellular portion of the chimeric protein, thereby activating the chimeric receptor. Known downstream proteins involved in intracellular signaling can then be assayed for expected modifications i.e. phosphorylation. Other methods known to those in the art can also be used to identify signaling molecules involved in receptor activity.

4.2.11 Metabolic Activity

The cachexia wasting disorder is characterized by progressive weight loss and depletion of lean body mass. Alterations in protein, fat, and carbohydrate metabolism occur commonly. For example, abnormalities in carbohydrate metabolism include increased rates of total glucose turnover, increased hepatic gluconeogenesis, glucose intolerance and elevated glucose levels. Increased lipolysis, increased free fatty acid and glycerol turnover, hyperlipidemia, and reduced lipoprotein lipase activity are frequently noted. The weight loss associated with cachexia is caused not only by a reduction in body fat stores, but also be a reduction in total body protein mass with extensive skeletal muscle wasting. Increased protein turnover and poorly regulated amino acid oxidation may also be important.

hC1Q/TNF7 polypeptides and polynucleotides can have an effect on regulating carbohydrate, fat and/or protein metabolism or turnover, thereby reversing the loss of body mass. Methods of measuring carbohydrate, fat and protein turnover in vitro and in vivo are well-known in the art and can be used to study the activity of hC1Q/TNF7 polypeptides and polynucleotides. Both stable and radioactive isotope tracers, including but not limited to ²H, ³H, ¹³C, ¹⁴C, ¹⁵N, and ¹⁸O, are typically used to measure a turnover rate (Kelleher, Metab. Eng. 3:100-110 (2001); Patterson, Metabolism 46:322-329 (1997) both of which are herein incorporated by reference in their entirety). The turnover of a compound can be determined by administering a tracer either as a bolus injection, a constant infusion, or a primed-infusion to a subject, for example a mouse (reviewed in McCabe and Previs, Metab. Eng. 6:25-35 (2004) herein incorporated by reference in its entirety). For example, [³H]- or [¹⁴C]-labeled 2-deoxy-D-glucose (a glucose analog) can be used to trace glucose utilization and the metabolic products can be analyzed in blood, adipose tissue and muscle (McCabe and Previs, 2004, supra; Colwell et al., Int. J. Biochem. Cell Biol. 28:115-121 (1996); Ferre et al., Biochem. J. 228:103-110 (1985); Furler et al., Am. J. Physiol. 255:E806-E811 (1998); Kragen et al., Am. J. Physiol. 248:E353-E362 (1985); Sokoloff et al., J. Neurochem. 28:897-916 (1977) all of which are herein incorporated by reference in their entirety). Deuterium-labeled water can be used to measure rates of synthesis and breakdown of triglycerides (Jensen et al., Am. J. Physiol. Endocr. Metab. 281:E998-E1004 (2001); Previs et al., Diabetes 50:A333 (2001) both of which are herein incorporated by reference in their entirety) and proteins (Previs et al., Diabetes 50:A301 (2001) herein incorporated by reference in its entirety). Protein degradation can be measured by directly weighing muscles excised from sacrificed experimental animals and measuring tyrosine release (Todorov et al., J. Biol. Chem. 272:12279-12288 (1997) herein incorporated by reference in its entirety). ELISA (enzyme-linked immunoabsorbant assay) assays can be used to measure circulating levels of molecules involved in metabolism, such as free fatty acids, leptin, insulin, etc.

hC1Q/TNF7 can also affect energy expenditure, appetite and caloric intake. Caloric intake is assayed by measurement of daily food intake according to established protocols. Body composition can be analyzed using several methods well known in the art. In one method, administration of ²H₂O or H₂¹⁸O is used to calculate total body water. Once total body water is measured, fat mass can be estimated and compared to experimental values (McCabe and Previs, 2004, supra; Annegers, Proc. Soc. Exp. Biol. Med. 87:454-456 (1954); Dawson et al., Comp. Biochem. Physiol. 42B:679-691 (1972) all of which are herein incorporated by reference in their entirety). Energy expenditure can be measured using doubly labeled water (²H₂O and H₂¹⁸O) and assaying oxygen consumption (Lifson et al., J. Appl. Physiol. 7:704-710 (1955); McClintock and Lifson, Am. J. Physiol. 189:463-469 (1957); Schoeller, J. Nutr. 129:1765-1768 (1999); Speakman, Am. J. Clin. Nutr. 68:932S-938S (1998) all of which are herein incorporated by reference in their entirety).

Cachexia increases the expression of uncoupling proteins (UCPs) which are involved in metabolism control. hC1Q/TNF7 polypeptides and polynucleotides can alter the expression or activity of UCPs thereby affecting a change in the subject's metabolism. UCP mRNA expression can be measured using standard protocols for Northern blot analysis or PCR amplification. UCP activity can be assayed using a yeast expression system (Gong et al., J. Biol. Chem. 272:24129-24132 (1997) herein incorporated by reference in its entirety).

4.2.12 Drug Screening

The non-human mammals expressing hC1Q/TNF7 of the present invention provide several important uses that will be readily apparent to one of ordinary skill in the art. In addition, mice that express hC1Q/TNF7 polypeptides as a result of injection with hC1Q/TNF7-transfected cells are also useful for screening compounds that modulate (i.e. increase or decrease) the activity of hC1Q/TNF7 polypeptides. Screening for a useful compound involves administering the candidate compound over a range of doses to the animal expressing hC1Q/TNF7, and assaying at various time points for the effect(s) of the compound on the activity of the hC1Q/TNF7 protein. The compound may be administered prior to or at the onset of abdominal distension. Administration may be oral, or by suitable injection, depending on the chemical nature of the compound being evaluated. The cellular response to the compound is evaluated over time using appropriate biochemical and/or histological assays.

Sources for test compounds that may be screened for ability to bind to or modulate (i.e., increase or decrease) the activity of polypeptides of the invention include (1) inorganic and organic chemical libraries, (2) natural product libraries, and (3) combinatorial libraries comprised of either random or mimetic peptides, oligonucleotides or organic molecules.

Chemical libraries may be readily synthesized or purchased from a number of commercial sources, and may include structural analogs of known compounds or compounds that are identified as “hits” or “leads” via natural product screening.

The sources of natural product libraries are microorganisms (including bacteria and fungi), animals, plants or other vegetation, or marine organisms, and libraries of mixtures for screening may be created by: (1) fermentation and extraction of broths from soil, plant or marine microorganisms or (2) extraction of the organisms themselves. Natural product libraries include polyketides, non-ribosomal peptides, and (non-naturally occurring) variants thereof. For a review, see Science 282:63-68 (1998).

Combinatorial libraries are composed of large numbers of peptides, oligonucleotides or organic compounds and can be readily prepared by traditional automated synthesis methods, PCR, cloning or proprietary synthetic methods. Of particular interest are peptide and oligonucleotide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). For reviews and examples of peptidomimetic libraries, see Al-Obeidi et al., Mol. Biotechnol, 9(3):205-23 (1998); Hruby et al., Curr Opin Chem Biol, 1(1):114-19 (1997); Dorner et al., Bioorg Med Chem, 4(5):709-15 (1996) (alkylated dipeptides).

4.3 Diseases Amenable to hC1Q/TNF7 Therapy

In one aspect, the present invention provides pharmaceutical reagents and methods useful for treating diseases and conditions wherein a gain of body mass, especially lean body mass, is desired. hC1Q/TNF7 polypeptides are useful to treat diseases and disorders characterized by a loss of body mass or inhibition of growth. Specifically, hC1Q/TNF7 polypeptides are useful to treat or prevent diseases or conditions that include without limitation: wasting disorders, including but not limited to cachexia associated with chronic or end-stage illnesses including, but not limited to, cancer, AIDS, myopathies (including muscle protein metabolism), sepsis, inflammatory response syndromes, chronic liver disease (including liver cirrhosis), chronic lung disease (including COPD), advanced kidney disease, advanced cardiovascular disease, severe infections (including viral, bacterial, and protozoan), tuberculosis, inflammatory bowel disease (including Crohn's disease), autoimmune disease (including rheumatoid arthritis and SLE), long-term convalescence, coma; treatment-induced cachexia, including chemotherapy- and radiation-induced cachexia; eating disorders, including anorexia and bulimia; diseases associated with growth impairment, including genetic diseases of dwarfism, growth hormone deficiency (including adult and juvenile), inflammatory bowel disease (including Crohn's disease), inflammatory diseases (including juvenile idiopathic arthritis); ageing.

Comparisons of hC1Q/TNF7 mRNA and protein expression levels between diseased cells, tissue and corresponding normal samples are made to determine if the subject is responsive to hC1Q/TNF7 therapy. Methods for detecting and quantifying the expression of hC1Q/TNF7 polypeptide mRNA or protein use standard nucleic acid and protein detection and quantitation techniques that are well known in the art and are described in Sambrook, et al., Molecular Cloning: A Laboratory Manual, Cold Spring Harbor Laboratory, N.Y. (1989) or Ausubel, et al., Current Protocols in Molecular Biology, John Wiley & Sons, New York, N.Y. (1989), both of which are incorporated herein by reference in their entirety. Standard methods for the detection and quantification of hC1Q/TNF7 mRNA include in situ hybridization using labeled hC1Q/TNF7 riboprobes (Gemou-Engesaeth, et al., Pediatrics 109: E24-E32 (2002), herein incorporated by reference in its entirety), Northern blot and related techniques using hC1Q/TNF7 polynucleotide probes (Kunzli, et al., Cancer 94: 228 (2002), herein incorporated by reference in its entirety, herein incorporated by reference in its entirety), RT-PCR analysis using hC1Q/TNF7-specific primers (Angchaiskisiri, et al., Blood 99:130 (2002)), and other amplification detection methods, such as branched chain DNA solution hybridization assay (Jardi, et al., J. Viral Hepat. 8:465-471 (2001), herein incorporated by reference in its entirety), transcription-mediated amplification (Kimura, et al., J. Clin. Microbiol. 40:439-445 (2002)), microarray products, such as oligos, cDNAs, and monoclonal antibodies, and real-time PCR (Simpson, et al., Molec. Vision, 6:178-183 (2000), herein incorporated by reference in its entirety). Standard methods for the detection and quantification of hC1Q/TNF7 protein include western blot analysis (Sambrook, et al., Molecular Cloning: A Laboratory Manual, Cold Spring Harbor Laboratory, N.Y. (1989), Ausubel, et al., Current Protocols in Molecular Biology, John Wiley & Sons, New York, N.Y. (1989)), immunocytochemistry (Racila, et al., Proc. Natl. Acad. Sci. USA 95:4589-4594 (1998) supra), and a variety of immunoassays, including enzyme-linked immunosorbant assay (ELISA), radioimmuno assay (RIA), and specific enzyme immunoassay (EIA) (Sambrook, et al., Molecular Cloning: A Laboratory Manual, Cold Spring Harbor Laboratory, N.Y. (1989), Ausubel, et al., Current Protocols in Molecular Biology, John Wiley & Sons, New York, N.Y. (1989)).

The diseases and conditions treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle. For example, hC1Q/TNF7 polypeptides and polynucleotides can be used to stimulate growth of farm animals instead of currently used growth enhancement methods. Furthermore, hC1Q/TNF7 polypeptides and polynucleotides can be used to treat wasting disorders and growth disorders in non-human mammals.

In addition, hC1Q/TNF7 orthologs can be used to treat non-human mammals. Several of the hC1Q/TNF7 orthologs of the present invention are listed herein and were generated by searching primarily genomic and EST sequences using hC1Q/TNF7 and C1qTNF2 sequences as a probe. The hC1Q/TNF7 orthologs (both polypeptides and polynucleotides) are listed in the attached sequence listing: murine (SEQ ID NO: 16-19), rat (SEQ ID NO: 20-23), bovine (SEQ ID NO: 24-27), porcine (SEQ ID NO: 28-31), and chicken (SEQ ID NO: 32-37). Any of the hC1Q/TNF7 orthologs of the invention can be used to treat the same species (i.e. chicken hC1Q/TNF7 to treat chickens) or can be used cross-species (i.e. murine hC1Q/TNF7 to treat cattle).

4.3.1 Therapeutic Methods

The compositions (including polypeptide fragments, analogs, variants and antibodies or other binding partners or modulators including antisense polynucleotides) of the invention have numerous applications in a variety of therapeutic methods. Examples of therapeutic applications include, but are not limited to, those exemplified herein.

One embodiment of the invention is the administration of an effective amount of hC1Q/TNF7 polypeptides or other composition of the invention to individuals affected by a disease or disorder that can be treated the peptides of the invention. While the mode of administration is not particularly important, parenteral administration is preferred. An exemplary mode of administration is to deliver an intravenous bolus. The dosage of hC1Q/TNF7 polypeptides or other composition of the invention will normally be determined by the prescribing physician. It is to be expected that the dosage will vary according to the age, weight, condition and response of the individual patient. Typically, the amount of polypeptide administered per dose will be in the range of about 0.01 μg/kg to 100 mg/kg of body weight, with the preferred dose being about 0.1 μg/kg to 10 mg/kg of patient body weight. For parenteral administration, hC1Q/TNF7 polypeptides of the invention will be formulated in an injectable form combined with a pharmaceutically acceptable parenteral vehicle. Such vehicles are well known in the art and examples include water, saline, Ringer's solution, dextrose solution, and solutions consisting of small amounts of the human serum albumin. The vehicle may contain minor amounts of additives that maintain the isotonicity and stability of the polypeptide or other active ingredient. The preparation of such solutions is within the skill of the art.

4.3.2 Pharmaceutical Formulations

A protein or other composition of the present invention (from whatever source derived, including without limitation from recombinant and non-recombinant sources and including antibodies and other binding partners of the polypeptides of the invention) may be administered to a patient in need, by itself, or in pharmaceutical compositions where it is mixed with suitable carriers or excipient(s) at doses to treat or ameliorate a variety of disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors and various growth factors such as any of the FGFs, epidermal growth factor (EGF), platelet-derived growth factor (PDGF), transforming growth factors (TGF-α and TGF-β), insulin-like growth factor (IGF), keratinocyte growth factor (KGF), and the like, as well as cytokines described herein.

The pharmaceutical composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. Conversely, protein or other active ingredients of the present invention may be included in formulations of the particular cytokine, lymphokine, other hematopoietic factor, thrombolytic or anti-thrombotic factor, or anti-inflammatory agent to minimize side effects of the clotting factor, cytokine, lymphokine, other hematopoietic factor, thrombolytic or anti-thrombotic factor, or anti-inflammatory agent (such as IL-1Ra, IL-1 Hy1, IL-1 Hy2, anti-TNF, corticosteroids, immunosuppressive agents). A protein of the present invention may be active in multimers (e.g., heterodimers or homodimers) or complexes with itself or other proteins. As a result, pharmaceutical compositions of the invention may comprise a protein of the invention in such multimeric or complexed form.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other active ingredient of the present invention is administered to a mammal having a condition to be treated. Protein or other active ingredient of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein or other active ingredient of the present invention in combination with cytokine(s), lymphokine(s), other hematopoietic factor(s), thrombolytic or anti-thrombotic factors.

4.3.3 Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein or other active ingredient of the present invention used in the pharmaceutical composition or to practice the method of the present invention can be carried out in a variety of conventional ways, such as oral ingestion, inhalation, topical application or cutaneous, subcutaneous, intraperitoneal (IP), parenteral or intravenous (IV) injection. Intravenous administration to the patient is preferred.

Alternatively, one may administer the compound in a local rather than systemic manner, for example, via injection of the compound directly into the tissue, often in a depot or sustained release formulation.

In another embodiment, the implantation of cells producing hC1Q/TNF7 (cell therapy) into a subject in need of treatment for wasting disorders or for impaired growth is contemplated. Cells that do not normally express hC1Q/TNF7 or that express low levels of hC1Q/TNF7 may be modified to produce therapeutic levels of hC1Q/TNF7 by transformation with a polynucleotide that encodes hC1Q/TNF7. The cells may be of the same species as the subject, or may be derived from a different species. Preferably, the cells are derived from the subject in need of hC1Q/TNF7 therapy. Human or nonhuman cells may be implanted in a subject using a biocompatible, semi-permeable polymeric enclosure to allow release of hC1Q/TNF7 protein, or may be implanted directly without encapsulation.

The polypeptides of the invention are administered by any route that delivers an effective dosage to the desired site of action. The determination of a suitable route of administration and an effective dosage for a particular indication is within the level of skill in the art. Dosages can then be adjusted as necessary by the clinician to provide maximal therapeutic benefit.

4.3.4 Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein or other active ingredient of the present invention, and preferably from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein or other active ingredient of the present invention, and preferably from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counter ions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The amount of protein or other active ingredient of the present invention in the pharmaceutical composition of the present invention will depend upon the nature and severity of the condition being treated, and on the nature of prior treatments which the patient has undergone. Ultimately, the attending physician will decide the amount of protein or other active ingredient of the present invention with which to treat each individual patient. Initially, the attending physician will administer low doses of protein or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein or other active ingredient of the present invention per kg body weight. For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein or other active ingredient of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing or other active ingredient-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalcium phosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropylmethylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorption of the protein from the polymer matrix and to provide appropriate handling of the composition, yet not so much that the progenitor cells are prevented from infiltrating the matrix, thereby providing the protein the opportunity to assist the osteogenic activity of the progenitor cells. In further compositions, proteins or other active ingredient of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals, livestock, farm animals and thoroughbred horses, in addition to humans, are desired patients for such treatment with proteins or other active ingredient of the present invention. The dosage regimen of a protein-containing pharmaceutical composition to be used for the gain of fatty or lean body mass will be determined by the attending physician considering various factors which modify the action of the proteins, e.g., amount of body mass desired to be increased, the severity of the disease or disorder, the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

4.3.5 Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p.1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 μg/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

4.3.6 Diagnostic Assays and Kits

The present invention further provides methods to identify the presence or expression of one of the ORFs of the present invention, or homolog thereof, in a test sample, using a nucleic acid probe or antibodies of the present invention, optionally conjugated or otherwise associated with a suitable label.

In general, methods for detecting a polynucleotide of the invention can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

4.3.7 Screening Assays

Using the isolated proteins and polynucleotides of the invention, the present invention further provides methods of obtaining and identifying modulatory agents which bind to a polypeptide encoded by an ORF corresponding to the nucleotide sequence set forth in SEQ ID NO: 1-3, 5, 7, 9, or 11-14, or bind to a specific domain of the polypeptide encoded by the nucleic acid. In detail, said method comprises the steps of:

(a) contacting an agent with an isolated protein encoded by an ORF of the present invention, or nucleic acid of the invention; and

(b) determining whether the agent binds to said protein or said nucleic acid.

The modulatory agents may increase or decrease the growth activity of hC1Q/TNF7.

In general, such methods for identifying compounds that bind to a polynucleotide of the invention can comprise contacting a compound with a polynucleotide of the invention for a time sufficient to form a polynucleotide/compound complex, and detecting the complex, so that if a polynucleotide/compound complex is detected, a compound that binds to a polynucleotide of the invention is identified.

Likewise, in general, therefore, such methods for identifying compounds that bind to a polypeptide of the invention can comprise contacting a compound with a polypeptide of the invention for a time sufficient to form a polypeptide/compound complex, and detecting the complex, so that if a polypeptide/compound complex is detected, a compound that binds to a polynucleotide of the invention is identified.

Methods for identifying compounds that bind to a polypeptide of the invention can also comprise contacting a compound with a polypeptide of the invention in a cell for a time sufficient to form a polypeptide/compound complex, wherein the complex drives expression of a target gene sequence in the cell, and detecting the complex by detecting reporter gene sequence expression, so that if a polypeptide/compound complex is detected, a compound that binds a polypeptide of the invention is identified.

Compounds identified via such methods can include compounds which modulate the activity of a polypeptide of the invention (that is, increase or decrease its activity, relative to activity observed in the absence of the compound). Alternatively, compounds identified via such methods can include compounds which modulate the expression of a polynucleotide of the invention (that is, increase or decrease expression relative to expression levels observed in the absence of the compound). Compounds, such as compounds identified via the methods of the invention, can be tested using standard assays well known to those of skill in the art for their ability to modulate activity/expression.

The agents screened in the above assay can be, but are not limited to, peptides, carbohydrates, vitamin derivatives, or other pharmaceutical agents. The agents can be selected and screened at random or rationally selected or designed using protein modeling techniques.

For random screening, agents such as peptides, carbohydrates, pharmaceutical agents and the like are selected at random and are assayed for their ability to bind to the protein encoded by the ORF of the present invention. Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like, capable of binding to a specific peptide sequence, in order to generate rationally designed antipeptide peptides, for example see Hurby et al., Application of Synthetic Peptides: Antisense Peptides,” In Synthetic Peptides, A User's Guide, W.H. Freeman, NY (1992), pp. 289-307, and Kaspczak et al., Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs which rely on the same EMF for expression control. One class of DNA binding agents are agents which contain base residues which hybridize or form a triple helix formation by binding to DNA or RNA. Such agents can be based on the classic phosphodiester, ribonucleic acid backbone, or can be a variety of sulfhydryl or polymeric derivatives which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide and other DNA binding agents.

Agents which bind to a protein encoded by one of the ORFs of the present invention can be used as a diagnostic agent. Agents which bind to a protein encoded by one of the ORFs of the present invention can be formulated using known techniques to generate a pharmaceutical composition.

### 5. EXAMPLES

### Example 1

### Isolation of SEQ ID NO: 1 from a cDNA Libraries of Human Cells

The novel nucleic acid of SEQ ID NO: 1 was obtained from pools of human cDNA libraries prepared from various tissues, using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and a single representative clone was selected from each group for gel sequencing. The 5′ sequence of the amplified insert was then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. The insert of SEQ ID NO: 1 was described as a novel sequence in international publication WO 03/048326 A1 (SEQ ID NO: 157).

### Example 2

### Assemblage of SEQ ID NO: 2 and 3

The nucleic acid (SEQ ID NO: 2) of the invention was assembled from sequences that were obtained from a cDNA library by methods described in Example 1 above, and in some cases obtained from one or more public databases. The final sequence was assembled using the EST sequences as seed. Then a recursive algorithm was used to extend the seed into an extended assemblage, by pulling additional sequences from different databases (i.e. Nuvelo's database containing EST sequences, dbEST version 114, gbpri 114, and UniGene version 101) that belong to this assemblage. The algorithm terminated when there were no additional sequences from the above databases that would extend the assemblage. Inclusion of component sequences into the assemblage was based on a BLASTN hit to the extending assemblage with BLAST score greater than 300 and percent identity greater than 95%.

Nearest neighbor results for the assembled contig were obtained by a FASTA search against Genpept, using the FASTXY algorithm. FASTXY is an improved version of FASTA alignment which allows in-codon frame shifts. The nearest neighbor results showed the closest homologue for each assemblage from Genpept (and contain the translated amino acid sequences for which the assemblages encodes). The nearest neighbor results are set forth in Table 3 below:

TABLE 3

| SEQ ID | Accession | | Smith-Waterman | |
|---|---|---|---|---|
| NO: | No. | Description | Score | % Identity |
| 2 | X53556 | Bos taurus type X | 657 | 42.963 |
| | | collagen | | |

The predicted amino acid sequence for SEQ ID NO: 2, was obtained by using a software program called FASTY (University of Virginia) which selects a polypeptide based on a comparison of translated novel polynucleotide to known polynucleotides (W. R. Pearson, Methods in Enzymology, 183:63-98 (1990), incorporated herein by reference). For SEQ ID NO: 2, the predicted start and stop nucleotide locations are listed in Table 4:

TABLE 4

| | Predicted beginning | |
|---|---|---|
| | nucleotide location | Predicted end nucleotide |
| | corresponding to first amino | location corresponding to |
| SEQ ID | acid residue of amino acid | first amino acid residue of |
| NO: | sequence | amino acid sequence |
| 2 | 142 | 1058 |

Using PHRAP (Univ. of Washington) or CAP4 (Paracel), a full-length gene cDNA sequence and its corresponding protein sequence were generated from the assemblage. Any frame shifts and incorrect stop codons were corrected by hand editing. During editing, the sequence was checked using FASTY and BLAST against Genbank (i.e. dbEST version 114, gbpri 114, UniGene version 101, Genpept release 124). Other computer programs which may have been used in the editing process were phredPhrap and Consed (University of Washington) and ed-ready, ed-ext and cg-zip-2 (Nuvelo, Inc.). The full-length nucleotide and amino acid sequences are shown in the Sequence Listing as SEQ ID NO: 2 and 3, respectively.

In order to express hC1Q/TNF7 (SEQ ID NO: 4), the full-length HC1Q/TNF7 DNA was PCR amplified from Marathon-ready cDNA libraries (Clontech). The primary PCR product was further amplified using nested PCR primers that generated hC1Q/TNF7 polypeptide when expressed in suitable cell lines, as described below in Example 5.

### Example 3

### Expression of hC1Q/TNF7 mRNA in Human Tissues

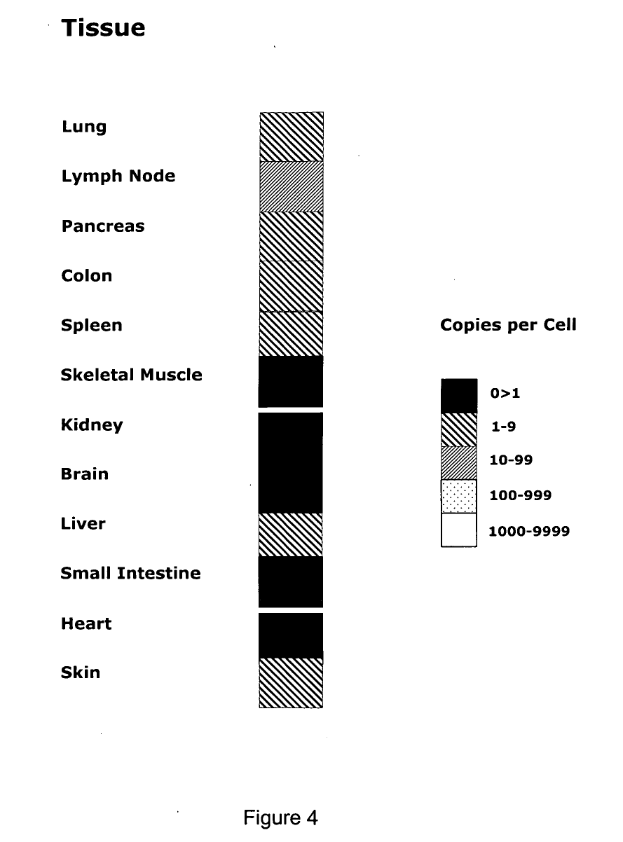

FIG. 4 shows the relative expression of hC1Q/TNF7 mRNA that was derived from human tissues.

Total mRNA was derived from the tissues indicated in FIG. 4 according to the protocol provided by the manufacturer (Qiagen, Valencia, Calif.). The RNA was subjected to quantitative real-time PCR (TaqMan) (Simpson et al., Molec Vision 6:178-183 (2000)) to determine the relative expression of hC1Q/TNF7 in the tissues shown. The forward and reverse primers that were used in the PCR reactions of human RNA were: 5′-AGGGAACTGCAGGTTTGAGA-3′ (forward; SEQ ID NO: 11), and 5′-TCCTCTGTCTCCCTTTGGTC-3′ (reverse; SEQ ID NO: 12), respectively. DNA sequences encoding Elongation Factor 1, β-actin, and ATP synthase 6 were used as a positive control and normalization factors in all samples. All assays were performed in triplicate with the resulting values averaged.

The graph shows the number of copies of hC1Q/TNF7 mRNA per cell assuming that each cell has 400,000 mRNA transcripts of a median length of 1.2 Kb, and that 2% of the total RNA in a cell is mRNA. FIG. 4 shows that hC1Q/TNF7 mRNA is expressed at low levels in all the tissues tested. The highest levels of hC1Q/TNF7 mRNA were seen in lymph node.

### Example 4

### Structural Modeling of hC1Q/TNF7

A three-dimensional structural model of the hC1Q/TNF7 protein was generated using the GeneAtlas™ software package (Accelrys, San Diego, Calif. 1999). This model was predicted based on a search of 4250 non-redundant Protein Data Bank structures using a PSI-BLAST multiple alignment sequence profile-based searching method (Meyers and Miller, Comput. Appl. Biosci. 4:11-17 (1988) herein incorporated by reference in its entirety) and high throughput homology modeling, an automated sequence and structure searching procedure (Sali and Overington, Protein Sci. 3:1582-1596 (1994) herein incorporated by reference in its entirety). The known crystal structure of adiponectin (Shapiro and Scherer, 1998, supra) was identified as the best fit structure and was used as a template for structural overlays using Profiles-3D, a threading program that measures the compatibility of the protein model with its sequence using a 3-D profile. Using defined parameters, Profiles-3D computes a score for the model normalized by the length of the amino acid sequence.

### Example 5

### Expression of Recombinant hC1Q/TNF7 Protein in Mammalian Cells

A. Construction of hC1Q/TNF7 (FL) in pIntron for Mouse Studies

The cDNA encoding hC1Q/TNF7 (SEQ ID NO: 5) was cloned into the pcDNA/Intron (pIntron) vector using EcoRI and XbaI sites to generate carboxy-terminal V5-His6-tagged hC1Q/TNF7 (SEQ ID NO: 9). The mammalian expression vector pcDNA/Intron was obtained by genetically modifying the pcDNA3.1TOPO vector (Invitrogen Inc., Carlsbad, Calif.) by introducing an engineered chimeric intron derived from the pCI mammalian expression vector (Promega, Madison, Wis.). A DNA fragment including the CMV immediate early enhancer/promoter and a chimeric intron was generated by restriction digestion of the pCI vector with BgIII and KpnI. The resulting fragment was then ligated into BglII and KpnI digest pcDNA3.1 to create pIntron.

The hC1Q/TNF7 ORF of SEQ ID NO: 4 (SEQ ID NO: 5) was first cloned into pcDNA3.1/V5His-TOPO (Invitrogen) by PCR using the following forward 5′-TGTCCAGACAAGAGCCAAAG-3′ (SEQ ID NO: 13) reverse 5′-CTGGGTAGCTGGTTGTGATG-3′ (SEQ ID NO: 14), and the EcoRI-XbaI insert from pcDNA 3.1/V5His-TOPO that contains the entire hC1Q/TNF7 ORF was ligated into the modified pIntron vector to generate the pIntron construct.

To generate stable cell lines expressing hC1Q/TNF7, the following approach was used: 2-4 μg of plasmid expressing V5-His-tagged hC1Q/TNF7 was transfected into HEK293 cells (obtained from ATCC) using the Fugene (Roche, Palo Alto, Calif.) transfection reagent according to the manufacturer's instructions. The cells transfected with the hC1Q/TNF7-pIntron plasmids were allowed to express the protein that confers resistance to geneticin for 24-48 hours prior to placing the cells under selection. Selection was performed by culturing the transfected cells in 1.5-2 mg of geneticin (G418) for 3-4 months. Positive clones were selected and expanded. After a selection period of 2-3 weeks, the cells were tested for the production of V5-tagged hC1Q/TNF7 by western analysis using an anti-V5 antibody (Invitrogen) to detect the presence of the protein (i.e. the presence of a V5-positive band at approximately 35 kD).

B. Construction of hC1Q/TNF7 Proteins for Expression in HEK-293 Cells

The gene sequence encoding full-length hC1Q/TNF7 was amplified by PCR from a combined human cDNA pool using primers specific to the full-length hC1Q/TNF7 sequence: 5′-TCCTGCAGCACCCCACCATCTAAG-3′ (SEQ ID NO: 38) and 5′-ACCCCAGATTCAATCAGAGTGTTTACCACAG-3′ (SEQ ID NO: 39). The PCR product was further amplified and subcloned into Eco RI and Xba I sites of pIntron, a modified mammalian expression plasmid with genemycin (G418) as a selection marker, with a nucleotide sequence encoding V5His6-tag fused to the carboxyl-terminal of full-length hC1Q/TNF7 coding sequence. The sequence was confirmed by nucleotide sequencing.

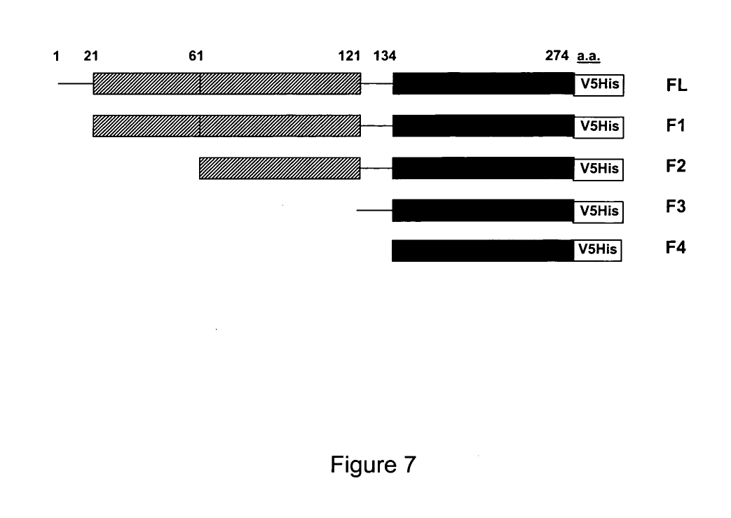

The PCR product corresponding to the coding sequence from amino acid position 21 to 274 (FIG. 7) was also amplified using primers: 5′-CCGGCTAGCCAACCCCGGGGTAATCAGTTG-3′ (SEQ ID NO: 40) and 5′-TGCTCTAGACTCAATTCATCATCTTCTGATATGGAATC-3′ (SEQ ID NO: 41), subcloned into the Nhe I and Xba I sites of pIntron.IgK, a modified expression vector with nucleotide sequence coding for human IgK signal peptide which was used as the signal peptide for the recombinant hC1Q/TNF7 protein expression in HEK-293 cells, and confirmed by nucleotide sequencing.

Similarly, PCR products corresponding to each truncated hC1Q/TNF7 protein (FIG. 7) were amplified using the primers listed in Table 5, subcloned into Nhe I and Xba I sites of pIntron.IgK, and confirmed by sequencing.

TABLE 5

| | | |
|---|---|---|
| Primer | Sequence | SEQ ID NO: |

| | | |
|---|---|---|
| hC1Q/TNF7 F1 | CCGGCTAGCCCTGGCTTGCCTGGACC | 42 |
| forward (IgK) | | |
| hC1Q/TNF7 F2 | CCGGCTAGCGGAACTGCAGGTTTGAGAGGTAAG | 43 |
| forward (IgK) | | |
| hC1Q/TNF7 F3 | CCGGCTAGCGGAGTTTGCAGATGTGGAAGCATC | 44 |
| forward (IgK) | | |
| hC1Q/TNF7 F4 | CCGGCTAGCGCCTTTTCTGTTGGCATCACAACC | 45 |
| forward (IgK) | | |
| hC1Q/TNF7 | GATTCCATATCAGAAGATGATGAATTGAGTCTAGAGCA | 46 |
| reverse for all | | |

C. Construction of hC1Q/TNF7 Proteins for Bacterial Expression

DNA sequences encoding full-length hC1Q/TNF7 (amino acid 21 to 274) and each truncation form of the protein were amplified by PCR using the primer sets corresponding to each protein (see Table 6). Each PCR product was cloned into the E. coli expression vector pCRT7-CT-TOPO (Invitrogen), with V5His6-tag fused to the C-terminal of the protein, and confirmed by DNA sequencing.

TABLE 6

| | | |
|---|---|---|
| Primer | Sequence | SEQ ID NO: |

| | | |
|---|---|---|
| hC1Q/TNF7 FL | ATGCAACCCCGGGGTAATCAGTTG | 47 |
| forward | | |
| hC1Q/TNF7 | CAATTCATCATCTTCTGAT | 48 |
| reverse for | | |
| all | | |
| hC1Q/TNF7 F1 | ATGCCTGGCTTGCCTGGACCTCCA | 49 |
| forward | | |
| hC1Q/TNF7 F2 | ATGGGAACTGCAGGTTTGAGAGGT | 50 |
| forward | | |
| hC1Q/TNF7 F3 | ATGGGAGTTTGCAGATGTGGAAGC | 51 |
| forward | | |
| hC1Q/TNF7 F4 | ATGGCCTTTTCTGTTGGCATCACA | 52 |
| forward | | |

D. Expression of hC1Q/TNF7 Recombinant Proteins in HEK-293 Cells

HEK-293 cells were transfected with the mammalian expression plasmids as described above using FuGene 6 transfection reagent (Roche). The transfected cells were selected with G418 at a concentration of 1 mg/ml. The stably selected cells were then subjected to clonal selection. The conditioned medium from each clonal cell line was screened for secretion of the expressed protein. The clonal cell lines with high protein expression level were expanded, suspension and serum-deficiency adapted, and used for the recombinant protein production.

E. Expression of hC1Q/TNF7 Recombinant Proteins in Bacteria

Each protein was expressed in E. coli BL21 DE3 pLysS (Invitrogen). The lysed and clarified lysate from bacterial pellets containing expressed proteins were used for protein isolation by Ni²⁺ affinity chromatography as described below. The collected eluate was dialyzed against PBS, and then subjected to Hi-Trap Q HP ion exchange column (Amersham) to remove potential endotoxin contaminants.

### Example 6

### Biological Analysis of hC1Q/TNF7 in vivo

Positive pools of cells expressing V5-His-tagged hC1Q/TNF7 were used for in vivo analysis after a 2-3 weeks of antibiotic selection. To facilitate expression in vivo in mice, the cell line HEK293 was selected for transfection for its ability to form tumors in immuno-compromised animals (i.e. nude mice). Stable bulk pools of cells were expanded and harvested to provide enough cells for the administration of 20-30 million cells per mouse. V5-His-tagged hC1Q/TNF7-expressing bulk pools of cells were administered subcutaneously to 8 week old Nu/Nu (Charles River, Mass.) mice on the left hind flank. Each experiment was controlled using a GFP (green fluorescent protein) HEK293 stable bulk pool. Expression of GFP is not known to result in the alteration of biological processes. V5-His-hC1Q/TNF7-expressing HEK293 mice were allowed to develop tumors for 3-4 weeks. To assess the levels of circulating V5-His-hC1Q/TNF7 protein, mice were bled retro-orbitally. The blood was then processed to obtain the serum component and analyzed by anti-V5 western analysis to determine the level of V5-His-hC1Q/TNF7.

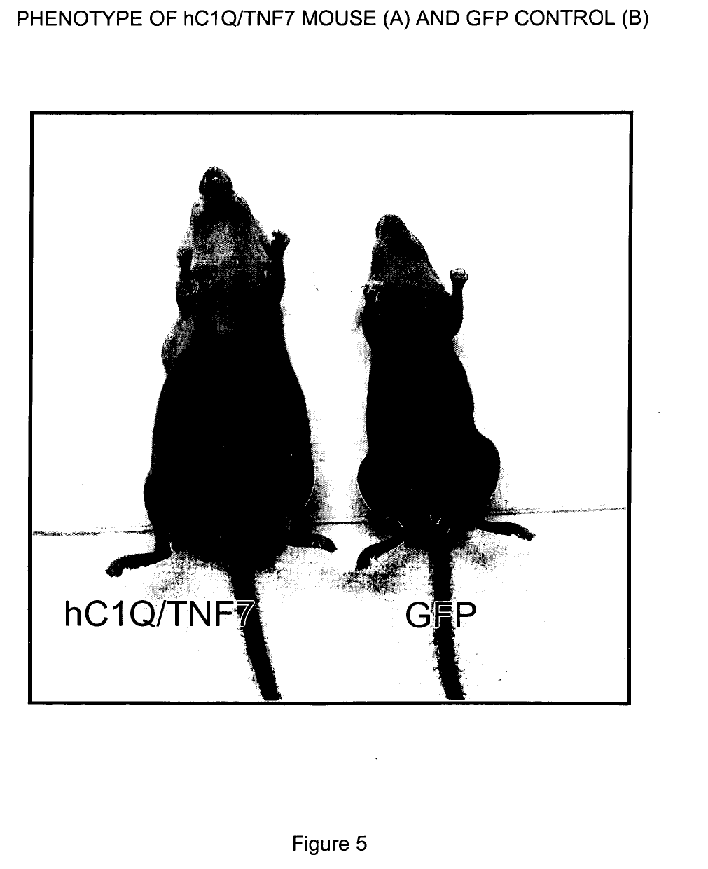

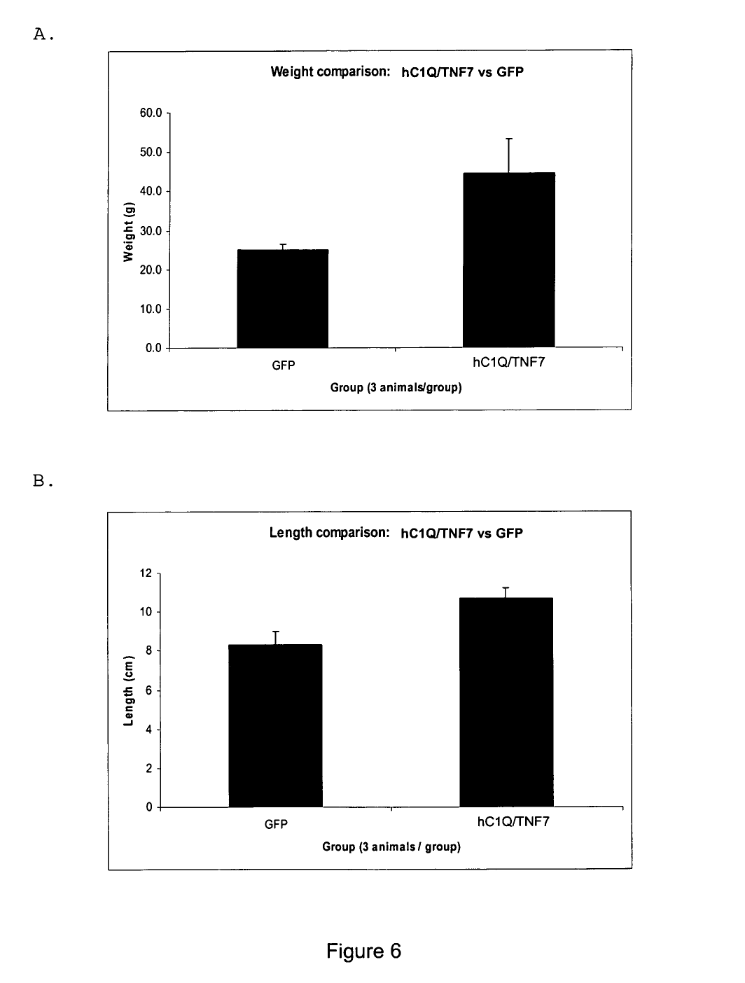

As can be seen in FIG. 5, the hC1Q/TNF7 expressing mice (B) are considerably larger than the GFP control (A). The hC1Q/TNF7 mice weigh more (FIG. 6A) and are longer (FIG. 6B) than the GFP controls.

Mice were euthanized and processed for analysis by removing as much blood as possible and harvesting the following organs: lungs, liver, heart, kidney, spleen, colon, small intestine, skin, and tumor. IDEXX Laboratories (West Sacramento, Calif.) processes the blood and tissue samples from each animal for downstream analysis. Serum chemistry analytes including, albumin, alkaline phosphatase, amylase, bilirubin D, bilirubin Id, bilirubin T, BUN (blood urea nitrogen), cholesterol, creatine, GGT (gamma glutamyltransferase), glucose, LDH (lactate dehydrogenase), protein T, ALT (alanine transaminase), AST (aspartate aminotransferase), triglycerides, and CBC (complete blood count) are analyzed. Tissue isolated from the above-mentioned organs is processed for H&E (hematoxylin and eosin) staining and is reviewed by IDEXX pathologists. The hC1Q/TNF7 tissue sections and serum chemistry analytes are compared to the GFP control.

### Example 7

### Purification of Recombinant hC1Q/TNF7

A. Purification of hC1Q/TNF7-F1 Protein from Mammalian 293 HEK Cells

hC1Q/TNF7 V5-His-F1 protein was expressed in 293 HEK cells grown in 293 free-style media with addition of 0.5% FBS. The cell growth culture was harvested and the supernatant was stored at −80° C.

Concentration and Dilfiltration—The frozen culture supernatant (−80° C.) containing secreted hC1Q/TNF7 V5-His-F1 protein was thawed at 4° C. Protease inhibitors EDTA and Pefbloc (Roche) were added to the media to a final concentration of 1 mM and 0.4 mM, respectively. The media was filtered through a 0.22 μm PES filter (Corning). Media was concentrated down to 10-fold using TFF system (Pall Filtron) with 10 kDa cut-off membrane. The concentrated media was buffer exchanged with 20 mM sodium phosphate, 0.5M NaCl, pH 7. After media concentration/diafiltration, mammalian protease inhibitor cocktail (Sigma) was added at 1:500 (v/v) dilution.

Ni-chelating Affinity Chramotography—A HiTrap Ni-chelating affinity column (Pharmacia) was equilibrated with 20 mM sodium phosphate, pH 7, 0.5 M NaCl. The buffer-exchanged media was filtered with 0.22 μm PES filter and loaded onto Ni-chelating affinity column. Ni Column was washed with 20 mM imidazole for 15 Column Volume (CV) and protein was eluted with a gradient of 20 mM to 300 mM imidazole over 30 CV. The fractions were analyzed by SDS-PAGE and Western blot. Fractions containing hC1Q/TNF7 V5-His-F1 protein were pooled and analyzed to be 35% pure by a Comassie stained gel under reduced condition. The pooled protein solution was concentrated to 1 mg/mL and dialyzed against 20 mM sodium phosphate, 0.15M NaCl, pH 7 (PBS). The final protein solution in PBS buffer was passed through a sterile 0.22 μm filter and stored at −80° C. The purified hC1Q/TNF7 V5-His-F1 protein from 293 HEK cells was injected into mice to examine the biological function of the protein.

B. Purification of hC1Q/TNF7 V5-His Protein Fragments from E. coli:

Purification of hC1Q/TNF7-V5-His Full Length Protein from E. coli Soluble Lysate

Extraction from E. coli Soluble Supernatant—E. coli cell paste containing hC1Q/TNF7 V5-His protein was resuspended in 25 mM Hepes, pH 7 at ratio of 1 g paste to 10 mL buffer. To prevent the degradation during the purification process, protease inhibitors cocktail for purification of His-tagged proteins (Sigma) were added at ratio of 1 mL per 20 g cell paste. Break the cells by passing through a homogenizer at pressure of 18000-20000 psi. Spin the whole cell lysate at 14,300×g for 40 minutes to separate supernatant and pellet. The soluble supernatant was kept and the pellet (insoluble inclusion body) was removed. The protein solution was passed through a BioCap 30 delipidate filter capsules (Cuno) to remove insoluble components, nucleic acid, lipids, cell debris and endotoxins.

PEI precipitation and Ammonion Sulfate Fractionation—The PEI precipitation process was used to remove negative charged DNA and endotoxins in soluble lysate. To minimize the protein of interest loss during PEI precipitation, NaCl was added to the resulting supernatant to make final concentration of 0.5 M NaCl in 25 mM Hepes, pH 7. 5% PEI (polyethylimine) stock solution pH 7 was added to the soluble lysate solution to a final concentration of 0.02%. The solution was incubated at 4° C. for 30 minutes and centrifuged at 14,000×g for 30 minutes to remove the precipitate.

Ammonium Sulfate solid was added slowly to the resulting supernatant with constant stirring to final concentration of 40% of saturation. The solution was stirred for 1 hour at 4° C. and then centrifuged at 14,000×g for 20 minutes. The spin pellet was collected and resuspended into 25 mM Hepes, 0.5 M NaCl, pH 7 buffer to solubilize the pellet.

Ni-chelating Affinity Chramotography—A HiTrap Ni-chelating affinity column (Amersham) was equilibrated with 25 mM Hepes, pH 7, 0.5 M NaCl. The resuspended protein solution was filtered with 0.22 μm PES filter and loaded onto Ni-chelating affinity column. The Ni Column was washed with 20 mM imidazole for 15 Column Volume (CV) and protein was eluted with a gradient of 20 mM to 300 mM imidazole over 30 CV. The fractions were analyzed by SDS-PAGE and Western blot using an anti-V5 antibody (Invitrogen). Fractions containing hC1Q/TNF7 V5-His were pooled to yield a protein solution that was 15% pure when analyzed by Comassie staining of an SDS-gel.

Hydrophobic Interaction Chromatography (Butyl Sepharose)—A Butyl Sepharose HIC column was equilibrated with 25 mM Hepes, pH 7, 0.3 M Ammonium Sulfate. The Ni pool protein solution was diluted and buffer exchanged to 25 mM Hepes, 0.3 M Ammonium Sulfate, pH 7 and loaded onto a Butyl Sepharose HIC column. Butyl column was washed with 10 CV 0.3 M AmSO₄ and eluted with a gradient of 0.3 M to 0 M Ammonium Sulfate over 15 CV. Fractions containing hC1Q/TNF7 V5-His were pooled to yield a protein solution that was 25% pure when analyzed by Comassie staining of an SDS-gel.

Anion Exchange Chromatography (Q Sepharose)—A Q-sepharose column was equilibrated with 25 mM Hepes, pH 7. The HIC pool protein solution was buffer exchanged to 25 mM Hepes, pH 7 and loaded onto an anion exchange Q-sepharose column. Q column was washed with 10 CV 25 mM Hepes, pH 7 buffer and eluted with a gradient of 0 M to 0.8 M NaCl, 25 mM Hepes, pH 7 over 25 CV. Fractions containing hC1Q/TNF7 V5-His were pooled to yield a protein solution that had a final 35% purity. The purified protein solution was concentrated to 1 mg/mL and dialyzed against 20 mM sodium phosphate, 0.15M NaCl, pH 7 (PBS). The final protein solution in PBS buffer was passed through a sterile 0.22 μm filter and stored at −80° C. The activity of purified hC1Q/TNF7 V5-His full length protein was tested with the cell-based bioassay (see Example 6). The endotoxin level of final purified protein was analyzed using the LAL enzymatic assay kit (Charles River). The results are summarized in Table 7.

Purification of hC1Q/TNF7-V5-His F2 and F3 Protein from E. coli Soluble Lysate

The procedures for extraction of hC1Q/TNF7-F2 and 3 proteins from E. coli soluble lysate followed by Cuno delipidate filter filtration and PEI precipitation were the same as described as above.

Ni-chelating Affinity Chramotography—A HiTrap Ni-chelating affinity column (Amersham) was equilibrated with 25 mM Hepes, pH 7, 0.5 M NaCl. The supernatant protein solution was filtered with 0.22 μm PES filter and loaded onto a Ni-chelating affinity column. Ni column was washed with 20 mM imidazole for 15 Column Volume (CV) and protein was eluted with a gradient of 20 mM to 500 mM imidazole over 40 CV. The fractions were analyzed by SDS-PAGE and Western blot using an anti-V5 antibody (Invitrogen). Fractions containing hC1Q/TNF7 V5-His were pooled and purity were analyzed to be 51% for fragment 2 and 60% for fragment 3, respectively, by Comassie staining of an SDS-gel.

Anion Exchange Chromatography (Q Sepharose)—A Q-sepharose column was equilibrated with 25 mM Hepes, pH 7. The Ni pool protein solution was buffer exchanged to 25 mM Hepes, pH 7 and loaded onto an anion exchange Q-sepharose column. Q column was washed with 10 CV 25 mM Hepes, pH 7 buffer and eluted with a gradient of 0 M to 1 M NaCl, 25 mM Hepes, pH 7 over 30 CV. Fractions containing hC1Q/TNF7 V5-His were pooled to yield a protein solution that was 70% purity for hC1Q/TNF7 fragment 2 and 85% purity for fragment 3, respectively. The purified protein solution was concentrated to 1 mg/mL and dialyzed against 20 mM sodium phosphate, 0.15M NaCl, pH 7 (PBS). The final protein solution in PBS buffer was passed through a sterile 0.22 μm filter and stored at −80° C. The activity of purified hC1Q/TNF7 V5-His fragment 2 and 3 proteins was tested with cell-based bioassay. The endotoxin level of final purified protein was analyzed using the LAL enzymatic assay kit (Charles River). The results are summarized in Table 7.

Purification of 9452-V5-His Fragment 2 Protein from E. coli Insoluble Inclusion Body

Extraction from Inclusion Body—The procedure for resuspension of cell paste, cell breakage and spin separation of the soluble supernatant and insoluble inclusion body are the same as described above. The soluble supernatant was removed and the pellet (insoluble inclusion body) was kept and washed with 25 mM Hepes, 2M urea, pH 7 buffer. After wash, the remaining pellet was solubilized with 100 mL of 25 mM Hepes, 6M urea, pH 7 buffer at 4° C. overnight. The solubilized protein solution was passed through a Cuno delipidate filter to remove DNA, lipids, and endotoxins and followed by passing through a 0.22 μm PES filter.

Ni-chelating Affinity Chramotography—A HiTrap Ni-chelating affinity column (Amersham) was equilibrated with 25 mM Hepes, pH 7, 6 M urea. The solubilized inclusion body protein solution was loaded onto Ni-chelating affinity column. The Ni column was washed with 20 mM imidazole for 15 Column Volume (CV) and protein was eluted with a gradient of 20 mM to 500 mM imidazole over 30 CV. The fractions were analyzed by SDS-PAGE and Western blot using an anti-V5 antibody (Invitrogen). Fractions containing hC1Q/TNF7 V5-His were pooled and purity was analyzed to be 70% by Comassie staining of an SDS-gel.

Anion Exchange Chromatography (Q Sepharose)—A Q-sepharose column was equilibrated with 25 mM Hepes, pH 8, 6M urea. The Ni pool protein solution was buffer exchanged to 25 mM Hepes, pH 8, 6M urea and loaded onto an anion exchange Q-sepharose column. Q column was washed with 10 CV 25 mM Hepes, pH 8 buffer and eluted with a gradient of 0 M to 0.7 M NaCl, 25 mM Hepes, pH 8 over 40 CV. Fractions containing hC1Q/TNF7 V5-His were pooled to yield a protein solution that was 80%. The Q pH 8 pool was concentrated and buffer exchanged to 25 mM MES, pH 4, 6M urea. The protein solution was loaded on a Q-sepharose column equilibrated with 25 mM MES, pH 4, 6M urea. hC1Q/TNF7 fragment 2 protein was collected and recovered in the Q flow through (column condition: pH 4). This step was performed to remove endotoxins and other impurities by passing through a Q column at pH 4.

Cation Exchange Chromatography (SP Sepharose)—A SP-sepharose column was equilibrated with 25 mM MES, pH 5, 6M urea. The Q pH 4 flow through protein solution was titrated to 25 mM MES, pH 5, 6M urea and loaded onto a cation exchange SP-sepharose column. SP column was washed with 10 CV 25 mM MES, pH 5 buffer and eluted with a gradient of 0 M to 1 M NaCl, 25 mM MES, pH 5, 6M urea over 20 CV. Fractions containing hC1Q/TNF7 fragment 2 protein were pooled to yield a protein solution that was 90%. The purified protein solution was concentrated to 1 mg/mL and dialyzed against 20 mM sodium phosphate, 0.15M NaCl, pH 7 (PBS). The final protein solution in PBS buffer was passed through a sterile 0.22 μm filter and stored at −80° C. The activity of purified hC1Q/TNF7 V5-His fragment 2 and 3 proteins was tested with cell-based bioassay.

C. Summary

Table 7 summarizes the purification process, final purity and final endotoxin level of individual fragments and domains of hC1Q/TNF7 V5-His protein from E. coli and mammalian 293 HEK cells. Since reduction of the endotoxin level is important, some of the purified protein from E. coli source was further passed through an endotoxin removal ActiClean gel (Sterogene). The endotoxin level after the ActiClean gel is also recorded in the table. However, most of the case, the protein recovery is relatively too low (<40%), thus is not included in the purification process.

TABLE 7

| | | | Final | Endotoxin |
|---|---|---|---|---|
| Protein | Host Cell | Purification Process | Purity | Level (EU/mg) |
| 9452 | E. coli soluble | PEI -> AmSO4 fractionation -> | 35% | 2500 |
| full length | lysate | Ni -> HIC -> Q pH 7 | | |
| 9452 Fg 2 | E. coli soluble | PEI -> Ni -> Q pH 7 | 70% | 80000 |
| | lysate | Q -> sterogene ActiClean gel | | 5000 |
| 9452 Fg 3 | E. coli soluble | PEI -> Ni -> Q pH 7 | 85% | 1300 |
| | lysate | Q -> sterogene ActiClean gel | | 140 |
| 9452 Fg 2 | E. coli inclusion | Ni -> Q pH 8 -> Q pH 4 -> SP | 90% | 400 |
| | body | pH 5 | | |
| 9452 D1 | 293 HEK | Ni pH 7 | 35% | 0.3 |

### Example 8

### Characterization of hC1Q/TNF7 Proteins

A. hC1Q/TNF7 Activity Assay

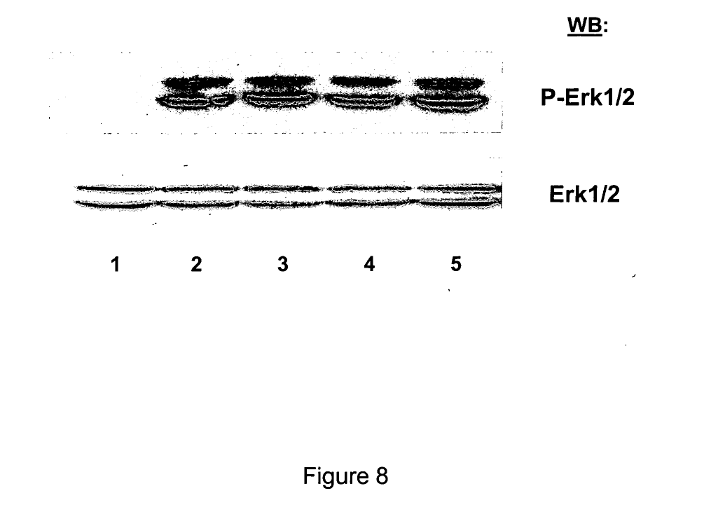

C2C12 cells (ATCC) grown in DMEM plus 10% FBS were washed with PBS and treated with 0.5 μg/ml of various hC1Q/TNF7 proteins for 30 minutes as indicated in FIG. 8. The treated cells were lysed in 1×Lysis Buffer (Cell Signaling Technology, Beverly, Mass.) in the presence of protease inhibitors (Roche) on ice for 1 hour. The clarified cell lysate was separated on a 10% Tris-Glycine SDS-PAGE, transferred to PVDF membrane and detected with phospho-Erk1/2-specific antibodies or anti-Erk1/2 antibodies (Cell Signaling Technology).

Full-length and truncated proteins all revealed similar potency in the induction of AMPK, ACC and Erk1/2 phosphorylation (FIG. 8). Therefore, in the Examples described below, HEK293-expressed full-length hC1Q/TNF7, hC1Q/TNF7-F1 and bacteria-expressed hC1Q/TNF7-F4 were further characterized.

B. Oligomerization Status

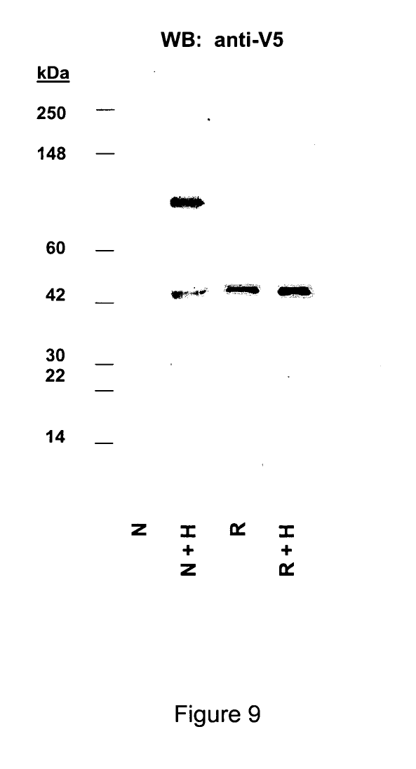

Studies of C1Q and 1C1Q-related proteins, such as adiponectin (Ad), indicate that most of the proteins form trimer, hexamer and higher molecular weight species (Shapiro and Scherer, Curr. Biol. 8:335-338 (1998) herein incorporated by reference in its entirety). To determine the oligomerization status of the hC1Q/TNF7 protein, the HEK293-expressed full-length hC1Q/TNF7 was treated under various conditions: non-reduced only (N); non-reduced plus heating at 100° C. for 10 min (N+H); reduced only (R); and reduced plus heating at 100° C. for 10 min (R+H). As shown in FIG. 9, under non-reducing conditions without heating, the protein migrated as two minor bands with mobilties of approximately 80 kDa and approximately 120 kDa, while most of the recombinant hC1Q/TNF7 protein accumulated on the top of the gel with a mobility higher than 250 kDa (Lane 1). After heat treatment under non-reducing conditions, hC1Q/TNF7 migrated in 3 major bands: approximately 40, approximately 80, and approximately 160 kDa (Lane 2). In contrast, when hC1Q/TNF7 was treated under reducing conditions with or without heating, it ran as a single band at approximately 40 kDa (Lanes 3 and 4). Similar results were obtained using hC1Q/TNF7-F1 protein and an N-terminal V5His-tagged version of full-length hC1Q/TNF7 (not shown). These data suggest that under natural conditions, hC1Q/TNF7 exists as dimers, tetramers, and higher order oligomers.

C. Glycosylation Status

To determine whether hC1Q/TNF7 is post-translationally glycosylated, the mobility between mammalian and bacterially expressed hC1Q/TNF7 proteins was compared. Determination of the glycosylation status of hC1Q/TNF7 was performed using an enzymatic deglycosylation kit from ProZyme (San Leandro, Calif.). Briefly, aliquots of the purified hC1Q/TNF7-F1 protein were treated with PNGase F, PNGase F+Sialidase A, or PNGase F+Sialidase A+O-Glycanase under denaturing conditions at 37° C. for 3 hours, or up to 3 days under non-denaturing conditions following the manufacturer's instructions. The treated samples were then separated on a 4-20% SDS-PAGE under reducing conditions and detected by Western blot using an HRP-conjugated anti-V5 antibody (Invitrogen).

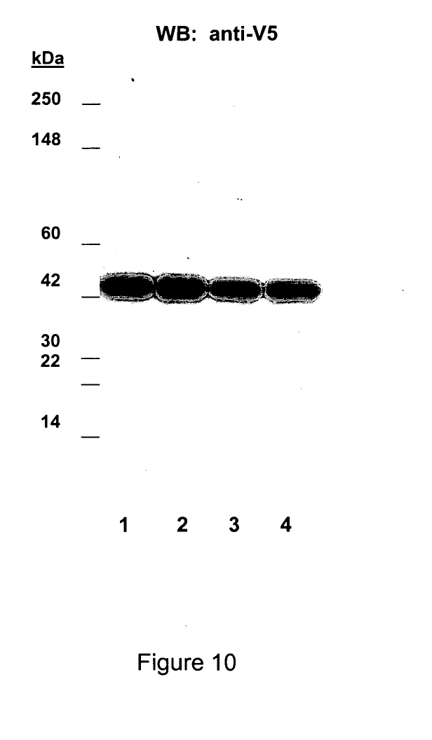

Comparison of recombinant proteins from these two expression systems revealed no detectable difference in mobility on SDS-PAGE. Furthermore, when hC1Q/TNF7-F1 purified from HEK293 conditioned media was treated with PNGase F as well as in combination with Sialidase A and O-Glycanase under denaturing conditions, there was no detectable mobility shift of the protein (see FIG. 10). Similar results were obtained when the protein was treated under non-denaturing conditions (not shown). Thus, the mature hC1Q/TNF7 is not post-translationally glycosylated.

C. Stability of hC1Q/TNF7

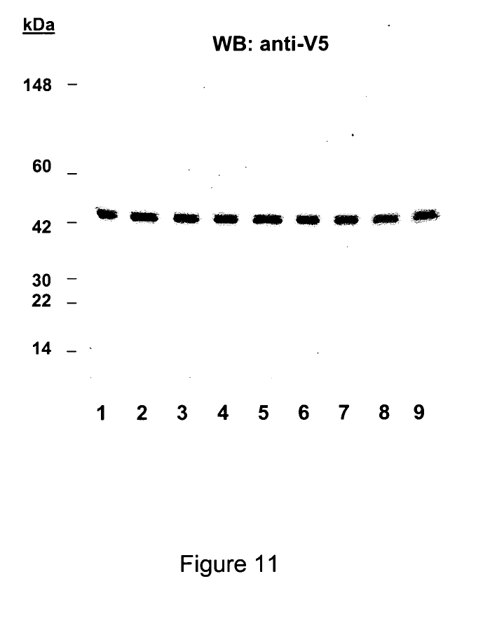

Purified hC1Q/TNF7-F1 protein was stored at 4° C. or 37° C. for different times as indicated in FIG. 11 (0, 1, 3, 4, or 5 days). The stability of the protein was determined by separating the protein on a 4-20% SDS-PAGE under reducing conditions and detecting with HRP-conjugated anti-V5 antibody. Storage at 4° C. for up to 5 days did not result in a detectable loss or degradation of the protein, while storage at 37° C. revealed minor loss of the protein at day 5. Thus, recombinant hC1Q/TNF7 protein is relatively stable.

### Example 9

### Activation of AMPK and ACC Signaling Pathways

A. AMPK Pathway

C2C12 cells grown in DMEM plus 10% FBS were washed with PBS and treated with hC1Q/TNF7-F1 protein as described below. Treated cells were lysed in 1×Lysis Buffer (Cell Signaling Technology) in the presence of protease inhibitors (Roche) on ice for 1 hour. The clarified cell lysate was then separated on a 4-20% Tris-Glycine SDS-PAGE, transferred to PVDF membrane and detected with anti-phospho-AMPK and anti-AMPK antibodies (Cell Signaling).

In FIG. 12A, purified hC1Q/TNF7-F1 protein was treated as follows: untreated (C); heat-inactivated (H-I); depleted with Ni2+ beads(Ni-D); or depleted with anti-V5 antibodies (V5-D). The remaining hC1Q/TNF7-F1 protein in each sample was detected using an anti-V5 antibody (bottom panel). C2C12 myoblasts were incubated with each sample protein for 20 min. Phosphorylated AMPK (top panel) as well as AMPK (middle panel) was detected using anti-phospho-AMPK and anti-AMPK antibodies, respectively.

Treatment of C2C12 myoblasts with hC1Q/TNF7-F1 significantly increased AMPK phosphorylation, an indication of activation of the AMPK signaling pathway (see FIG. 12A, Lane 2). To exclude the possibility that this effect was caused by contaminants, the same protein preparation was subjected to heat inactivation or depletion using either anti-V5 antibody or Ni²⁺ beads (see FIG. 12A, Lanes 3-5). Heat inactivation (Lane 3) completely blocked the stimulation of AMPK phosphorylation. Similarly, immuno-depletion by anti-V5 antibody precipitation completely depleted the protein resulting in the disrupted ability to activate AMPK phosphorylation (Lane 5). The hC1Q/TNF7-F1 protein preparation from Ni²⁺ bead depletion partially stimulated AMPK phosphorylation (Lane 4, top panel) since depletion by Ni²⁺ beads only partially depleted the protein (Lane 4, bottom panel). Similarly, hC1Q/TNF7-F1 also induced AMPK phosphorylation in 82-HTB cells, a human rhabodomyosarcoma cell line (not shown).

To determine the time-course of AMPK activation, C2C12 myoblasts were treated with hC1Q/TNF7-F1 for different times (FIG. 12B). The clarified cell lysates were resolved on a 4-20% Tris-Glycine SDS-PAGE. Phosphorylated AMPK and AMPK were detected with anti-phospho-AMPK and anti-AMPK antibodies, respectively. hC1Q/TNF7-F1 rapidly stimulated AMPK phosphorylation within 10 min and the stimulated AMPK phosphorylation was sustained for up to 2 hours (FIG. 12B). In a control experiment, globular adiponectin (gAd, R&D Systems, Minneapolis, Minn.) also rapidly stimulated AMPK phosphorylation, however, the increased AMPK phosphorylation diminished after 30 min of treatment (not shown), which is consistent with results previously reported (Yamauchi et al., Nat. Med. 11:1288-1295 (2002) herein incorporated by reference in its entirety).

To determine the potency of hC1Q/TNF7-F1 on AMPK activation, C2C12 myoblasts were treated with different concentrations of hC1Q/TNF7-F1 for 30 min and detected as stated above. As shown in FIG. 12C, hC1Q/TNF7-F1 increased AMPK phosphorylation in a dose-dependent fashion. The increased AMPK phosphorylation reached a maximum at 200 ng/ml.

B. ACC Pathway

C2C12 myoblasts were incubated with recombinant hC1Q/TNF7-F1 protein at 0, 0.1, 0.25, 0.5 and 1.0 μg/ml for 30 min. The cell lysate was clarified and resolved on a 4-20% Tris-Glycine SDS-PAGE. Western blot analysis was performed using anti-phospho-ACC antibodies (Cell Signaling Technology) (FIG. 13A, top panel). The cell lysates were also blotted with anti-β-actin to estimate the loading of each sample (FIG. 13A, bottom panel). Western blot analysis on cell lysates from hC1Q/TNF7-F1 and PBS-treated C2C12 myoblasts was performed as described above. The quantity of normalized phospho-ACC at each time point was estimated by scanning densitometry using the Image program (National Institutes of Health) and expressed as a percentage of maximum phospho-ACC content at 30 min after treatment (FIG. 13B).

Similar to that of AMPK, hC1Q/TNF7 also rapidly induced ACC phosphorylation with high potency and sustained effect. Therefore, hC1Q/TNF7 activates AMPK and ACC signaling pathways.

### Example 10

### Activation of the MAPK Signaling Pathway

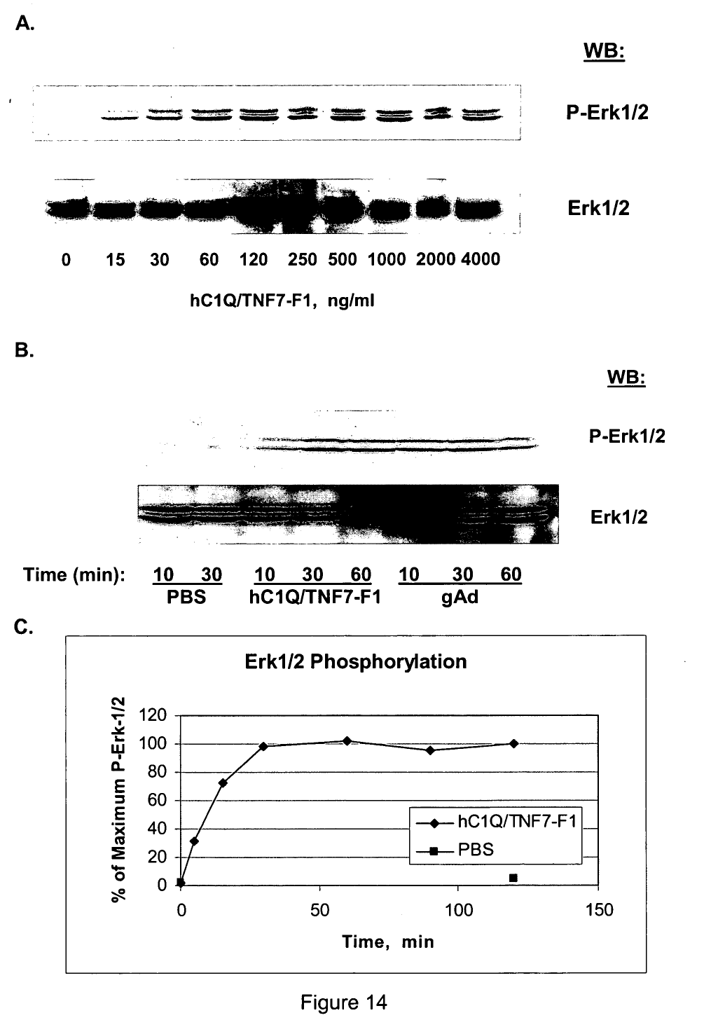

The effect of hC1Q/TNF7 on Erk1/2 phosphorylation was examined. C2C12 myoblasts were incubated with hC1Q/TNF7-F1 protein at 0, 15, 30, 60, 120, 250, 500, 1000, 2000, or 4000 ng/ml (FIG. 14A). Erk1/2 phosphorylation was determined by Western blot analysis using phospho-specific Erk1/2 antibodies (top panel, Cell Signaling Technology). Equal loading was confirmed by Western blot using anti-Erk1/2 antibodies (bottom panel, Cell Signaling Technology). The duration of Erk1/2 phosphorylation induced by hC1Q/TNF7-F1 was determined by incubating C2C12 myoblasts with PBS, hC1Q/TNF7-F1 (1 μg/ml for 10, 30, or 60 min), or gAd (2 μg/ml for 10, 30, or 60 min) as indicated in FIG. 14B and analyzed as above. The timecourse of hC1Q/TNF7-F1 phosphorylation of Erk1/2 was estimated by scanning densitometry using the Image program and expressed as a percentage of maximum phospho-Erk1/2 content at 30 min after treatment.

hC1Q/TNF7-F1 rapidly stimulated Erk1/2 phosphorylation within 10 min and the induced Erk1/2 activation lasted for 2 hours (see FIG. 14B and C), whereas gAd-induced Erk1/2 phosphorylation diminished after 60 min of treatment (FIG. 14B). Moreover, hC1Q/TNF7-F1 activated the Erk1/2 pathway with high potency and significantly stimulated Erk1/2 phosphorylation at 15 ng/ml (FIG. 14A). In control experiments, treatment of C2C12 myoblasts with up to 2 μg/ml of hC1Q/TNF7 did not induce activation of p38 MAPK, c-Jun N-terminal kinase or insulin receptor substrate-1 (not shown). Thus, hC1Q/TNF7-F1 specifically activates Erk1/2 MAPK pathway in a rapid and sustained fashion.

### Example 11

### Stimulation of Glucose Uptake

Confluent C2C12 myoblasts grown in 24-well plates in DMEM plus 10% FBS were washed with HBS buffer (20 mM HEPES, 140 mM NaCl, 1 mM CaCl₂, 5 mM KCl, 2.5 mM MgSO₄) plus 10 mM D-glucose and incubated with the same buffer at 37° C. for 2 hours. Cells were washed and incubated in HBS buffer without D-glucose containing PBS, different amounts of hC1Q/TNF7-F1 (HEK-293 expressed), hC1Q/TNF7-F4 (bacteria expressed), gAd, human insulin (Sigma), or 5 μM Cytochalasin B as a background control, as indicated in FIG. 15A and incubated at 37° C. for 30 min. 2-deoxy-D-[1-³H]glucose (Amersham) in the absence or presence of 10 mM glucose was then added to each well and incubated at 37° C. for 60 min. The cells were washed with ice-cold PBS plus 10 mM glucose three times, solubilized with 0.05% SDS at 4° C. for at least 15 min, and radioactivity was counted in a liquid scintillation counter.

Both mammalian and bacterially expressed hC1Q/TNF7 significantly stimulated glucose uptake in C2C12 myoblasts in a dose-dependent fashion with a maximum increase at approximately 50% (FIG. 15A), which was comparable to the stimulating effect by human insulin, a well-known stimulator of glucose uptake in muscle cells (Combs et al., J. Clin. Invest. 108:1875-1881 (2001) herein incorporated by reference in its entirety). Furthermore, treatment of myoblasts with a combination of hC1Q/TNF7 and insulin resulted in increased glucose uptake (approximately an 80-90% increase, FIG. 15B), suggesting that the effect on stimulating glucose uptake by insulin and hC1Q/TNF7 is additive. Moreover, treatment by either boiling or anti-V5 depletion completely abolished the stimulating effect of hC1Q/TNF7 on glucose uptake.

### Example 12

### Fatty Acid Oxidation Assay

Fatty acid oxidation was performed as described in Segall et al., Am. J. Physiol. 277:E521-E528 (1999) herein incorporated by reference in its entirety. Briefly, confluent C2C12 myoblasts grown in 24-well plates in DMEM plus 10% FBS were washed once with 1 ml incubation medium (DMEM, 12 mM glucose, 4 mM glutamine, 25 mM HEPES, 1% FFA-free BSA, 0.1 mM palmitate) and incubated in 0.5 ml of the same medium for 1 hour at 37° C. Recombinant hC1Q/TNF7-F1 (1 μg/ml), hC1Q/TNF7-F4 (1 μg/ml), gAd (2 μg/ml), or PBS was added to each well and incubated for 15 min at 37° C. [1-¹⁴C]Palmitate (0.05 μCi/well, Amersham) was added to each well, the plate was sealed and incubated at 37° C. for 90 min. At the end of the incubation, each well was sealed with a blue cap fitted with a piece of Whatman paper in the center. Each well was then injected with 75 μl of 6 M HCl (from the edge of the cap) using a syringe and 30 μl of 2 M NaOH was injected onto the Whatman paper. The plate was re-sealed and incubated at 37° C. for approximately 90 min. The Whatman paper from each well was transferred to a scintillation vial in the presence of 500 μl 0.128 M NaOH to which 5 ml scintillation liquid was added, incubated for at least 1 hour, and counted for radioactivity in a scintillation counter.

hC1Q/TNF7 significantly increased [1-¹⁴C]Palmitate oxidation by at least 50%, similar to that by gAd (FIG. 16). Therefore, AMPK activation by hC1Q/TNF7 increases ACC phosphorylation and consequently results in stimulated fatty acid oxidation.

### Example 13

### Analysis of hC1Q/TNF7 in Cancer Cachexia Models

Cachexia is induced in mice by subcutaneous implantation in the right flank of a vital fragment (approximately 2 mm in diameter) of the murine adenocarcinoma C26-B (see van Halteren et al, J. Cancer Res. Clin. Oncol. 2004 Jan. 27 (epub ahead of print) herein incorporated by reference in its entirety) Mice are treated daily with either HEK293 cells expressing hC1Q/TNF7 (as described in Example 6) or with at least 1 mg/kg/day of recombinant hC1Q/TNF7 that is prepared according to the method of Example 7). Treatment begins the day after tumor implantation (day 1) until day 11. Tumor volume, body weight, caloric intake, and hematocrit are measured. At day 17, the experiment is terminated, the mice are weighed, the tumor is harvested, blood is collected, and tissues, including the gastrocnemius muscle, interscapular brown adipose tissue (BAT), and gonadal fat pads, are rapidly excised, weighed, and frozen in liquid nitrogen. All hC1Q/TNF7 samples are compared to GFP controls.

Plasma levels of TNFα, IL-1β, IL-6, leptin, free fatty acids, cholesterol, triglyceride, phospholipids, insulin and glucose are determined using commercially available enzymatic colorimetric tests. Expression levels of the leptin receptor and neuropeptide Y are analyzed by isolating total RNA from frozen hypothalamus tissues and performing quantitative RT-PCR on said RNA using primers specific for the leptin receptor and neuropeptide Y (Bing et al., J. Neurochem. 79:1004-1012 (2001) herein incorporated by reference in its entirety).

Protein degradation is analyzed by quickly ligating and dissecting out the gastrocnemius muscles and placing them in ice-cold isotonic saline. Protein degradation is measured by tyrosine release according to the method outlined in Todorov et al. J. Biol. Chem. 272:12279-12288 (1997) herein incorporated by reference in its entirety).

Alternatively, protein turnover and degradation can be analyzed by measuring the decay in specific and total protein radioactivity in tibialis muscles after labeling in vivo, 24 h before tumor transplantation with a single intraperitoneal dose of sodium [¹⁴C]-bicarbonate (250 μCi/kg body weight) and then measuring the rates of protein degradation, synthesis, and accumulation after sacrifice (see method described in Carbó et al., Br. J. Cancer 83:526-531 (2000) herein incorporated by reference in its entirety).

The effect of hC1Q/TNF7 on uncoupling proteins (UCP-1, -2, and -3) is assayed by examining their mRNA expression and enzymatic activity. Total RNA is isolated from BAT and gastrocnemius muscle according to standard techniques. UCP-1, -2, and -3 mRNAs are detected by Northern blotting using specific probes (see Bing et al., Cancer Res. 60:2405-2410 (2000) herein incorporated by reference in its entirety). UCP activity is monitored using a yeast expression system and measuring mitochondrial membrane potential using the potential-sensitive dye, 3,3′-dihexyloxacarbobyanine iodide (see method described in Gong et al., J. Biol. Chem. 272:24129-24132 (1997) herein incorporated by reference in its entirety).

1. A method of increasing body mass in a subject comprising administering to said subject a composition comprising a hC1Q/TNF7 polypeptide, fragment, or analog thereof and a carrier.

2. A method of treatment comprising administering to a mammalian subject in need thereof a therapeutically effective amount of a composition comprising a hC1Q/TNF7 polypeptide and a pharmaceutically acceptable carrier.

3. A method of treating a metabolic disorder comprising administering to a mammalian subject in need thereof a therapeutically effective amount of a composition comprising a hC1Q/TNF7 polypeptide and a pharmaceutically acceptable carrier.

4. The method of claim 2, 3, or 4, wherein the mammalian subject is a human.

5. The method of claim 2, 3, or 4, wherein the hC1Q/TNF7 polypeptide is selected from the group consisting of SEQ ID NO: 4, 8, 10, 54, 56, 58, 60, 62, 64, 67, 69, 71, 73, or 75.

6. The method of claim 3, wherein the metabolic disorder is a wasting disorder.

7. A method of treating a metabolic disorder comprising administering to a mammalian subject in need thereof a therapeutically effective amount of a composition comprising a hC1Q/TNF7 polynucleotide and a pharmaceutically acceptable carrier.

8. The method of claim 7, wherein the hC1Q/TNF7 polynucleotide is selected from the group consisting of SEQ ID NO: 1-3, 7, 9, 11-14, 38-53, 55, 57, 59, 61, 63, 65-66, 68, 70, 72, or 74.

9. The method of claim 7, wherein the mammalian subject is a human.

10. The method of claim 7, wherein the metabolic disorder is a wasting disorder.

11. A method of treating a metabolic disorder comprising administering to a mammalian subject in need thereof a therapeutically effective amount of a composition comprising a host cell transformed or transfected with a hC1Q/TNF7 polynucleotide.

12. The method of claim 11, wherein the hC1Q/TNF7 polynucleotide is selected from the group consisting of SEQ ID NO: 1-3, 7, 9, 11-14, 38-53, 55, 57, 59, 61, 63, 65-66, 68, 70, 72, or 74.

13. The method of claim 11, wherein the mammalian subject is a human.

14. The method of claim 11, wherein the metabolic disorder is a wasting disorder.

15. A method of determining biological activity of hC1Q/TNF7 comprising
(a) transfecting an isolated mammalian cell with an expression vector that comprises hC1Q/TNF7 gene operatively linked to expression regulatory elements;
(b) selecting the transfected cells that express hC1Q/TNF7;
(c) injecting a non-mammalian animal with the cells of step (b); and
(d) observing the phenotype of said animal.
